(19) United States
(12) Patent Application Publication
(10) Pub. No.: US 2024/0261360 A1
(43) Pub. Date: Aug. 8, 2024

(54) NUTRACEUTICAL COMPOSITION

(71) Applicant: Metaceutic APS, Søborg (DK)

(72) Inventor: Ali Kazemi, Søborg (DK)

(21) Appl. No.: 17/999,730

(22) PCT Filed: May 21, 2021
(86) PCT No.: PCT/EP2021/063631

§ 371 Date: Nov. 23, 2022

(30) Foreign Application Priority Data
May 25, 2020 (DK) PA 2020 70336

(51) Int. Cl. A61K 36/81; A61K 9/00; A61K 31/4415; A61K 31/525; A61K 31/593; A61K 31/714; A61K 36/16; A61K 36/185; A61K 36/23; A61K 36/258; A61K 36/28; A61K 36/324; A61K 36/35; A61K 36/38; A61K 36/41; A61K 36/42; A61K 36/45; A61K 36/48; A61K 36/53; A61K 36/537; A61K 36/67; A61K 36/68; A61K 36/725; A61K 36/736; A61K 36/738; A61K 36/752; A61K 36/82; A61K 36/87; A61K 36/88; A61K 36/899; A61K 36/9066; A61K 36/9068; A61P 1/14

(52) U.S. Cl. CPC A61K 36/81; A61K 9/0053; A61K 31/4415; A61K 31/525; A61K 31/593; A61K 31/714; A61K 36/16; A61K 36/185; A61K 36/23; A61K 36/258; A61K 36/28; A61K 36/324; A61K 36/3486; A61K 36/35; A61K 36/38; A61K 36/41; A61K 36/42; A61K 36/45; A61K 36/48; A61K 36/53; A61K 36/537; A61K 36/5777; A61K 36/67; A61K 36/68; A61K 36/725; A61K 36/736; A61K 36/738; A61K 36/752; A61K 36/82; A61K 36/87; A61K 36/88; A61K 36/899; A61K 36/9066; A61K 36/9068; A61P 1/14

(57) ABSTRACT

The present invention relates to a nutraceutical composition, use of a nutraceutical composition, and a method of increasing or decreasing a relative population of bacteria in the intestines of a mammal. The composition comprises an extract containing two or more plant secondary metabolites, wherein said two or more plant secondary metabolites are encapsulated in an acid resistant coating. The composition can affect the composition of bacteria in the gut of mammals.

### FIELD OF THE INVENTION

The present invention relates to a nutraceutical composition, the use of one or more extracts, such as plant extract(s) as a nutraceutical composition, and a method of regulating (e.g., increasing or decreasing) one or more relative population(s) of bacteria in the intestines of a mammal.

### BACKGROUND ART

A healthy gut microbiota is characterized by a diverse population of bacteria abundancies. Most of the Human diseases affecting westernized countries are associated with dysbiosis and loss of microbial diversity in the gut microbiota. The Western way of life, with a wide use of antibiotics and other environmental triggers, may reduce the number of bacterial predators leading to a decrease in microbial diversity of the Human gut (Mosca A, Leclerc M, Hugot J P. Gut Microbiota Diversity and Human Diseases: Should We Reintroduce Key Predators in Our Ecosystem?. Front Microbiol. 2016; 7:455. Published 2016 Mar. 31. doi:10.3389/fmicb.2016.00455.) Prebiotics are substances in biologic material, such as e.g., food, that regulate the growth and/or activity of beneficial or non-beneficial microorganism in the gut's microbiota.

Prebiotics are often referred to as fibers, which are carbohydrates and digested by such microorganisms in the microbiota. Prebiotics are thus referred to as substances that can alter the composition of microorganism in the gut of mammals.

In contrast probiotics refer to live bacteria and yeasts that are present or enriched in a biologic material, such as e.g., food sources and delivered to the digestive system. Suitable composition of beneficial microorganisms in the gut are believed to strengthen the immune system, neutralise toxins, normalise bowel movements, control cholesterol, counter allergies and skin problems, and prevent microbial infections. Further, recent studies have shown that the composition of microorganisms in the gut are linked to an array of diseases, such as irritable bowel syndrome, dementia, attention deficit hyperactivity disorder (ADHD), anxiety, depression, mental stress, and cognitive disfunction. Known prebiotics include dietary fibres, such as polysaccharides and oligosaccharides. Mosca et al. 2016 suggest that administering probiotica is a simple therapeutic approach to reintroduce bacterial predators and restore a bacterial diversity of the host microbiota. (Mosca A, Leclerc M, Hugot J P. Gut Microbiota Diversity and Human Diseases: Should We Reintroduce Key Predators in Our Ecosystem?. Front Microbiol. 2016; 7:455. Published 2016 Mar. 31. doi:10.3389/fmicb.2016.00455).

Probiotics have an extensive history of safe use, particularly in healthy people. However, a risk of harmful effects from probiotics may be present to some degree in patients with severe illnesses or compromised immune systems. Another potential challenges for the gut health associated with the use of probiotics is a monoculture or non-diverse bacteria abundancy. Thus, probiotics cannot increase (or balance) the diversity of the gut microbiota, as they are generally focused on mono-cultures of a few selected strains. CA2447376C describes prebiotic and probiotic compositions and methods for their use in gut-based therapies. The composition may be microencapsulated or coated with an enteric coating. Furthermore, the document describes prebiotics as being typically thought of as carbohydrates of relatively short chain length, such as fructan oligosaccharide, araban oligosaccharide, inulin, lactulose, or other vegetable fibers. In addition, the document mentions that the prebiotics that seem to exert the best prebiotic effects are inulin-type fructans that are resistant to digestion by gastric acid and pancreatic enzymes in vivo.

However, there may be many other compounds that may act as prebiotics, e.g., other plant secondary metabolites than carbohydrates.

WO 201610985A1 discloses multi-supplement compositions comprising a probiotic supplement. Probiotics are generally understood to be bacteria or yeasts. The purpose of a probiotic supplement is increasing the relative population of the provided probiotic bacteria. It further discloses that unfortunately, probiotic strains are extremely sensitive, and some strains may not be able to survive commercial production, storage, or gastrointestinal transit after consumption where they are exposed to heat, moisture, bile, low pH, and digestive enzymes and that bacterial composition provided in WO 2016109856 A1 can be designed to release its contents when the composition reaches a location of the intestines having a pH above about 5.

Multiple studies have associated the abundance of blautia with type 1 diabetes, type 2 diabetes and higher visceral fat accumulation. WO 2019/008551 A2 discloses a composition for oral administration and for the treatment of diabetes. The composition comprises an extract from the costus plant and contains two plant secondary metabolites; triterpenoids and oxalic acid from said costus plant. This disclosure aims to increase the content of triterpenoids, which are claimed beneficial for the treatment of diabetes and to reduce the content of oxalic acid to below 1%. The final concentration of triterpenoids in said extract is 95% (w/w).

EP 3270707 B1 discloses a composition comprising apple, grape, green tea, and olive extracts. This synergistic formulation apple, grape, green tea, and olive extract are in amounts that provide a greater antioxidant activity or protein kinase modulating activity than provided by an equivalent amount of any one extract or a sum of the extracts.

Further, many individuals are challenged to eat the right amount of pro- and pre-biotics via their diet to effectively regulate the population of beneficial and non-beneficial bacteria in the gut to a healthy microbiota profile. Often, the amount of the food or ingredients, such as plant secondary metabolites needed directly in the gut, to regulate the desired microbiota diversity and thus bacteria abundancies, is not compatible with modern life. Creating prebiotic compositions to overcome these challenges and to facilitate a healthy gut microbiota in a convenient way are highly desirable to prevent the potential diseases and conditions resulting from a non-diverse or overly non-beneficial bacteria populated microbiota.

WO2016109856 disclosed multi-supplement composition comprising (a) a probiotic supplement; (b) a multivitamin and mineral supplement; (c) an antioxidant supplement; and (d) an anti-inflammatory supplement comprising acid resistant coatings. In formulation G it discloses compositions comprising SMs, wherein amino acids are the major SMs present.

Depending on the goal to be achieved, the relative abundance of one or more beneficial bacteria in the gut microbiota may be increased by the methods and compositions described herein, or the relative abundance of one or more bacteria having a negative impact on health may be decreased. Current knowledge is based on correlation between diseases or conditions and bacteria genus avunencies, and not causation. Thus depending on the context, the same bacteria genus abundancy may be to large or dominant or too small or depleted, compared to what is desirable or “normal”. This means that one bacteria genus may have positive impact on the health or wellbeing of a subject, but have negative impact on the same of another. Thus, in some cases, it would be especially desirable to produce a prebiotic composition which stimulate growth of beneficial bacteria, such as from the genera Bifidobacteria and/or Faecalibacteria and/or Bacteroides and/or Prevotella. Bifidobacteria are known to produce vitamins and short-chain fatty acids (SCFA) in the gut and may play a role in modulation of the immune system and in reduction of gut infection. Faecalibacterium comprises the species Faecalibacterium prausnitzii, a butyrate-producing bacterium, which plays a vital role in diabetes. Bacteroides ferment carbohydrates and produce SCFA. Also, low levels of Bacteroides are associated with inflammatory bowel disease. Prevotella are more abundant in people with plant-rich diets and is believed to improve glucose and insulin tolerance when associated with a diet rich in fibre. In other cases, or in addition, it would be especially desirable to produce a prebiotic composition which decreases the presence of bacteria that have a negative impact on health (i.e., non-beneficial bacteria), such as bacteria from the genus Blautia.

Preferably, the prebiotic composition both increases beneficial bacteria and decreases bacteria having negative impacts on health. Currently, no prebiotic compositions are known that increase the population of beneficial bacteria and decrease the population of non-beneficial bacteria at the same time. In some instances, it may be even more beneficial to combine the beneficial effects of prebiotic and probiotic compositions to regulate the microbiota to the desired diverse pattern of bacteria abundancies.

As previously mentioned, the most used prebiotics are fibres, which are carbohydrates digested by the microbiota's microorganisms. Other substances that are potent prebiotics, but which break down in the stomach acid before entering the intestines may however exist and not be applied as prebiotics as of now. At least because of these reasons, there remains an untapped potential in using many substances as prebiotics, including substances that are not carbohydrates, to stimulate a healthy composition of microorganisms in the gut.

### BRIEF DESCRIPTION OF THE FIGURES

In the following, embodiments of the present invention will be described with reference to the enclosed non-binding drawings.

FIG. 1 shows the % abundance of bacteria of the genus Bifidobacterium in faecal samples after treatment with extracts containing plant secondary metabolites.

FIG. 2 shows the % abundance of bacteria of the genus Bacteroides in faecal samples after treatment with extracts containing plant secondary metabolites.

FIG. 3 shows the % abundance of bacteria of the genus Faecalibacteria in faecal samples after treatment with extracts containing plant secondary metabolites.

FIG. 4 shows the % abundance of bacteria of the genus Prevotella in faecal samples after treatment with extracts containing plant secondary metabolites.

FIG. 5 shows the % abundance of bacteria of the genus Blautia in faecal samples of three individuals after treatment with an extract (FM-01) containing plant secondary metabolites.

### SUMMARY OF THE INVENTION

The invention is as defined in the appended claims.

In one embodiment a composition comprises an extract, said extract comprising at least two plant secondary metabolites, wherein said at least two plant secondary metabolites are encapsulated by an acid resistant coating and wherein each plant secondary metabolites constitutes less than about 50% (w/w) by dry weight of the extract. Said composition comprising an extract may be a mixture of several extracts and wherein said at least two plant secondary metabolites constitute at least 10% (w/w) by dry weight of the extract.

A composition as described herein may be used in treating or alleviating dysbiosis and/or loss of microbial diversity in the gut microbiota, which composition comprises an extract comprising at least two plant secondary metabolites, which are encapsulated in an acid resistant coating; wherein said extract is a mixture of several extracts.

In one embodiment a method of increasing or decreasing the relative population of one or more bacteria genus relative population in the intestines of a vertebrate animal, comprises the steps of:
selecting a genus or a species of intestinal bacteria genus to be increased or decreased;
selecting at least two plant secondary metabolites able to increase or decrease said genus or species of intestinal bacteria,
providing a composition comprising an extract containing said at least two plant secondary metabolites, wherein said at least two plant secondary metabolites constitute at least 10% (w/w) by dry weight of the extract, wherein said at least two plant secondary metabolites are encapsulated in an acid resistant coating;
orally administering the composition to the vertebrate animal.

A composition as described herein may be used in treating or alleviating dysbiosis and/or loss of microbial diversity in the gut microbiota which composition comprises an extract comprising at least two plant secondary metabolites, which are encapsulated in an acid resistant coating; wherein said extract is a mixture of several extracts.

In one embodiment a method of increasing or decreasing the relative population of one or more bacteria genus relative population in the intestines of a vertebrate animal, comprises the steps of:
selecting a genus or a species of intestinal bacteria genus to be increased or decreased;
selecting at least two plant secondary metabolites able to increase or decrease said genus or species of intestinal bacteria, wherein said plant secondary metabolites are selected from tables 2-5, preferably 3 or 5, based on the effect on the bacteria,
providing a composition comprising an extract containing said at least two plant secondary metabolites, wherein said at least two plant secondary metabolites constitute at least 10% (w/w) by dry weight of the extract, wherein said at least two plant secondary metabolites are encapsulated in an acid resistant coating;
orally administering the composition to the vertebrate animal.

In one embodiment said method comprises the step of selecting at least two plant secondary metabolites able to increase or decrease said genus or species of intestinal bacteria; wherein said plant secondary metabolites are selected from the list consisting of polyphenols, triterpenoids, glycosides, polysaccharides, terpenes, alkaloids, curcuminoids and polysaccharides, when an increase in a particular bacteria genus' population is desired.

In one embodiment said method comprises the step of selecting at least two plant secondary metabolites able to increase or decrease said genus or species of intestinal bacteria; wherein said plant secondary metabolites are selected from the list consisting of anthocyanidins, withanolides, flavonoids, triterpenoid saponins, beta glucan, saponins, proanthocyanidins, α-pinene, boswellic acids, theobromine, diarylheptanoids, phenolic acids, piperine, flavones, ginsenosides, saponins, gingerol, flavonoid glycosides and pectins, when an increase in a particular bacteria genus' population is desired.

In one embodiment said method comprises the step of selecting at least two plant secondary metabolites able to increase or decrease said genus or species of intestinal bacteria; wherein said plant secondary metabolites are selected according to which bacteria genus population is desired to be increased and on the highest possible correlation factor for said bacteria genus listed in Table 2, 3, 4 or 5, preferably Table 3 or 5. Said tables may be used as a guidance to find the plant secondary metabolites suitable for increasing or decreasing a particular bacteria genus population in the gut of a subject, such as a mammal (e.g. a human or animal).

In one embodiment said method comprises the step of selecting at least two plant secondary metabolites able to increase or decrease said genus or species of intestinal bacteria; wherein said plant secondary metabolites are selected according to which bacteria genus population is desired to be increased and on the highest possible correlation factor for said bacteria genus listed in Table 2 or 3, preferably Table 3.

In one embodiment said method comprises the step of at least two plant secondary metabolites able to increase or decrease said genus or species of intestinal bacteria; wherein said plant secondary metabolites are selected from the list consisting of polyphenols, polysaccharides, terpenes, alkaloids, glycosides, terpenes and curcuminoids, when a decrease in a particular bacteria genus' population is desired.

In one embodiment said method comprises the step of selecting at least two plant secondary metabolites able to increase or decrease said genus or species of intestinal bacteria; wherein said plant secondary metabolites are selected from the list consisting flavonoids, beta glucan, ginsenosides, flavones, theobromine, saponins, proanthocyanidins, diarylheptanoids, gingerol, boswellic acids, flavonoid glycosides, phenolic acids, saponins, piperine, anthocyanidins, α-pinene, withanolides, rosavins and inulin, when a decrease in a particular bacteria genus' population is desired.

In one embodiment said method comprises the step of selecting at least two plant secondary metabolites able to increase or decrease said genus or species of intestinal bacteria; wherein said plant secondary metabolites are selected according to which bacteria genus population is desired to be increased and on the highest possible correlation factor for said bacteria genus listed in Table 4 or 5, preferably Table 5.

In one embodiment, a composition as disclosed herein is resistant to dissolution below pH about 4, said composition comprising an extract and optionally one or more pharmaceutically or nutritionally acceptable excipients, wherein said extract is a mixture of several extracts, preferably a mixture of two or more extracts and wherein said mixture of two or more extracts comprises at least two plant secondary metabolites (hereinafter abbreviated SMs).

One or more of the above recited objectives and/or further objectives are achieved by one aspect of the present disclosure, wherein there is provided a composition for oral administration, the composition comprising an extract containing two or more SMs, wherein the SMs constitute at least 10% (w/w) by dry weight of the extract, which two or more SMs are encapsulated in an acid resistant coating. Other objectives and/or further objectives are achieved by one aspect of the present disclosure, wherein there is provided a composition, the composition comprising an extract containing two or more SMs, wherein the SMs constitute at least 10% (w/w) by dry weight of the extract, wherein said extract and/or composition is encapsulated in an acid resistant coating.

In one embodiment said composition dissolves at a pH above about 4, preferably pH about 4.8. In one embodiment said composition disintegrates at a pH above about 4, preferably pH about 4.5 or 4.8.

Further aspects relate to methods of using said compositions to regulate bacteria abundancy and microbiota diversity in the gut of a patient or mammal.

Depending on the goal to be achieved and the plant secondary metabolites selected, the relative abundance of one or more beneficial bacteria in the gut may be increased by the methods and compositions described herein, or the relative abundance of one or more bacteria having a negative impact on health may be decreased. In some embodiment plant secondary metabolites from specific sources, such as e.g., plant sources may be preferred to achieve a desired effect on the gut microbiota.

### DETAILED DESCRIPTION

The following are selected embodiments of the invention and should not be construed as limiting for the invention provided herein. The present invention will be described below relative to specific embodiments. Those skilled in the art will appreciate that the present invention may be implemented in several different applications and embodiments and is not specifically limited in its application to the particular embodiment depicted herein. Several compositions are referenced in the figures. Details about the compositions are listed below.

In one embodiment, a composition as disclosed herein is resistant to dissolution below pH about 4, said composition comprising an extract and optionally one or more pharmaceutically or nutritionally acceptable excipients, wherein said extract is a mixture of several extracts. In one embodiment, a composition as disclosed herein is resistant to dissolution below pH about 4, said composition comprising an extract and optionally one or more pharmaceutically or nutritionally acceptable excipients, wherein said extract is a mixture of several extracts, preferably a mixture of two or more extracts and wherein said mixture of two or more extracts comprises at least two plant secondary metabolites (SMs).

In one embodiment if one of said at least two secondary metabolites is a polysaccharide, then at least one other SMs in the extract is selected from the group consisting of: alkaloids, amino acids, curcuminoids, filling agent (not SM), glycosides, polyphenols, polysaccharides, terpenes and vitamins. In one embodiment said composition dissolves at a pH above about 4, preferably pH about 4.5 or 4.8. In one embodiment said composition disintegrates at a pH above about 4, preferably pH about 4.5 or 4.8. In one embodiment said composition does not dissolves at a pH below 4. In one embodiment said composition does not disintegrate at a pH below 4, preferably it disintegrates at pH above 4, preferably pH about 4.5 o 4.8.

One or more of the above recited objectives and/or further objectives are achieved by one aspect of the present disclosure, wherein there is provided a composition for oral administration, the composition comprising an extract containing two or more SMs, wherein the SMs constitute at least 10% (w/w) by dry weight of the extract. In one aspect, the composition comprises an extract containing two or more SMs, wherein the SMs constitute at least 10% (w/w) by dry weight of the extract, wherein at least two SMs are encapsulated by means preventing absorption of said SMs at pH below 4.

One or more of the above recited objectives and/or further objectives are achieved by one aspect of the present disclosure, wherein there is provided a composition for oral administration, the composition comprising an extract containing two or more SMs, wherein at least two SMs constitute at least 10% (w/w) by dry weight of the extract. One or more of the above recited objectives and/or further objectives are achieved by one aspect of the present disclosure, wherein there is provided a composition for oral administration, the composition comprising an extract containing two or more SMs, wherein at least two SMs constitute at least 10% (w/w) by dry weight of the extract and wherein none of the plant secondary metabolites constitutes of more than 50% (w/w) by dry weight of said extract.

One or more of the above recited objectives and/or further objectives are achieved by one aspect of the present disclosure, wherein there is provided a composition for oral administration, the composition comprising an extract containing two or more SMs, wherein at least two SMs constitute at least 10% (w/w) by dry weight of the extract and wherein each plant secondary metabolite constitute of 0-50% (w/w) by dry weight of said extract. In some embodiments each plant secondary metabolites constitute less than about 50% (w/w) by dry weight of said extract. Thus, in one embodiment each plant secondary metabolites constitute less than about 50% (w/w), such as 30% (w/w) by dry weight of said extract. In some embodiments each plant secondary metabolites constitute less than about 30% (w/w), such as 20% (w/w) by dry weight of said extract.

One or more of the above recited objectives and/or further objectives are achieved by one aspect of the present disclosure, wherein there is provided a composition for oral administration, the composition comprising an extract containing two or more SMs, wherein at least two SMs constitute at least 20% (w/w) by dry weight of the extract. One or more of the above recited objectives and/or further objectives are achieved by one aspect of the present disclosure, wherein there is provided a composition for oral administration, the composition comprising an extract containing two or more SMs, wherein at least two SMs constitute at least 25% (w/w) by dry weight of the extract. One or more of the above recited objectives and/or further objectives are achieved by one aspect of the present disclosure, wherein there is provided a composition for oral administration, the composition comprising an extract containing two or more SMs, wherein the SMs constitute at least 10% (w/w) by dry weight of the extract.

In one aspect the composition comprises an extract, said extract comprising at least two plant secondary metabolites, which are encapsulated in an acid resistant coating; wherein said extract is a mixture of several extracts. By providing an acid resistant composition comprising SMs said SMs can, when administered orally, at least partially be delivered to the intestines without being subjected to the stomach acids and thus be provided in higher amounts that naturally found in food stuff or other probiotics.

By coating the SMs with an acid resistant coating or providing other means to render the oral composition resistant to disintegration or dissolution at acidic pH, the SMs can, when administered orally, at least partially be delivered to the intestines without being subjected to the stomach acids.

A first effect of this is that at least some of the SMs susceptible to breakdown in stomach acid are not disintegrated by the stomach acid. Hence, a greater quantity of the SMs may be available to the microorganisms in the gut. This may be compared to SMs in foods, but also compared to SMs in other prebiotics.

Another effect is be that at least some of the SMs are not diluted in the gastric juice. As such, the nutraceutical composition may lead to a higher local concentration of SMs in the intestines, relative to when an uncoated extract was administered orally. Without being bound by theory, it is believed that a relatively high local concentration of SMs in the intestines made possible by the acid resistant coating is what leads to the nutraceutical composition's significant effect on growth of beneficial microorganisms in the gut.

The inventor has surprisingly found that an extract comprising at least two SMs that are provided directly to the gut, leads to a notable growth of beneficial microorganisms in the gut and/or a significant reduction in the abundance of bacteria associated with negative health impacts. The Examples illustrate these effects on selected bacteria genus.

The inventor has surprisingly found that an extract of some embodiments comprising at least two SMs that are provided directly to the gut, leads to a significant growth of beneficial microorganisms in the gut and/or a significant reduction in the abundance of bacteria associated with negative health impacts. The Examples illustrate these effects on selected bacteria genus. In one embodiment the effect of a composition on one or more specific bacteria abundancies may be analysed in an in vivo method comprising the steps of:
a) Collecting a first faeces sample from one, or preferable two or three healthy subjects, such as a mammal (e.g. human or animal) before administration of said composition,
b) Analysing the abundancy of selected bacteria genus in said sample, said sample will be the control sample,
c) Collecting a second faeces sample from a subject, such as a mammal (e.g. human or animal) before administration of said composition after administration of said one, or preferable two or three healthy subjects for about 15 days,
d) Analysing the abundancy of selected bacteria genus in said sample,
e) Normalizing the results of said second sample with said control sample, i.e. said first sample.

In one embodiment the step of analysing the abundancy of selected bacteria genus in said sample may be performed by an in vitro method comprising the step of:
a) Collecting a faeces sample from one, or preferable two or three healthy subjects, such as a mammal (e.g. human or animal) before administration of said composition,
b) Adding said sample in triplicated samples for control testing and composition testing to a simulated the human colon by means of controlling temperature (36.5° C.), anaerobic conditions, and pH (between 6-7) and a dedicated basal medium according to Wiese et al 2018 (DOI 10.7717/peerj.4268),
c) To the triplicated samples for control testing, a control substance, such as inulin is added,
d) To the triplicated sample for composition testing, the composition is added,
e) Analysing the bacteria abundancy at time 0 and time 24 for both control and composition test samples.

The abundancy in said faeces samples may be tested by a method consisting of the steps:
a) Isolating DNA,
b) Sequencing of said DNA to identify the bacteria genus and its quantity in said sample,
c) Normalize said sample to a control.

In one embodiment said composition is considered to have an increasing effect on the bacteria abundancy for a selected bacteria genus. This may be expressed using the log 2 fold change or a normalized percent (%)-wise change, between control sample and composition test sample. If expressed in log 2fold change, the increase or decrease should at least be about 0.1 for an increase or below about −0.1 for a decrease. If expressed in log 2fold change, the increase or decrease should preferably at least be about 0.5 for an increase or below about −0.2 for a decrease. If expressed in log 2fold change, the increase or decrease should preferably at least be about 0.2 for an increase or below about −0.5 for a decrease. If expressed in log 2 fold change, the increase or decrease should preferably at least be about 0.7 for an increase or below about −0.7 for a decrease. If expressed in log 2 fold change, the increase or decrease should preferably at least be 1 for an increase or below about 1 for a decrease.

If expressed in a percent-wise change, the increase or decrease should at least be about 10% for an increase or below about −10% for a decrease. If expressed in a percent-wise change, the increase or decrease should preferably at least be about 20% for an increase or below about −20% for a decrease. If expressed in a percent-wise change, the increase or decrease should preferably at least be about 30% for an increase or below about −30% for a decrease. If expressed in a percent-wise change, the increase or decrease should preferably at least be about 40% for an increase or below about −40% for a decrease. If expressed in a percent-wise change, the increase or decrease should preferably at least be about 50% for an increase or below about −50% for a decrease.

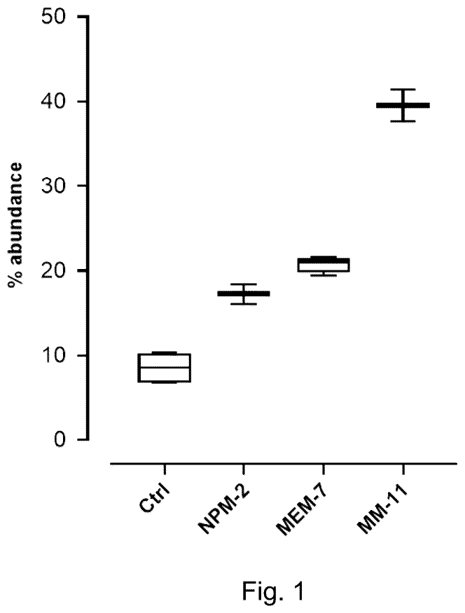

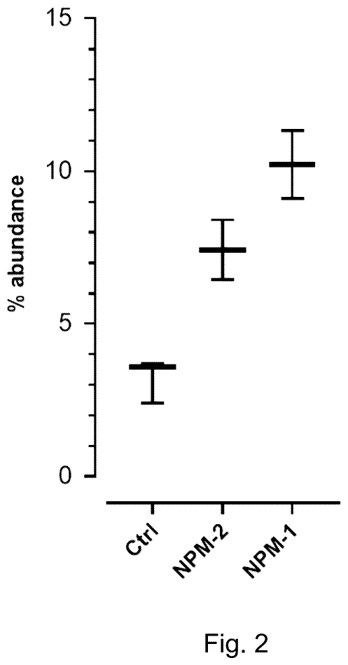

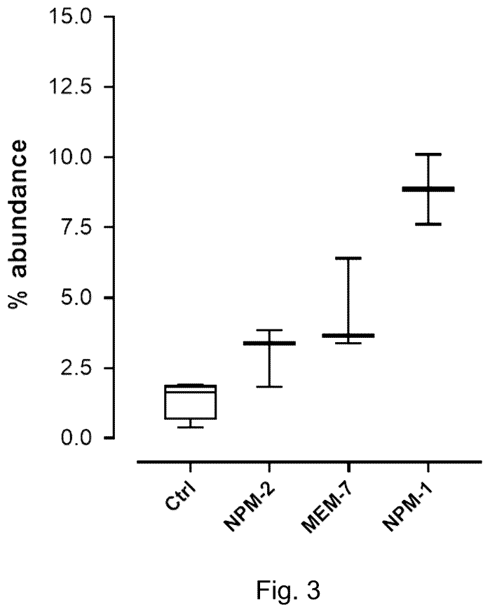

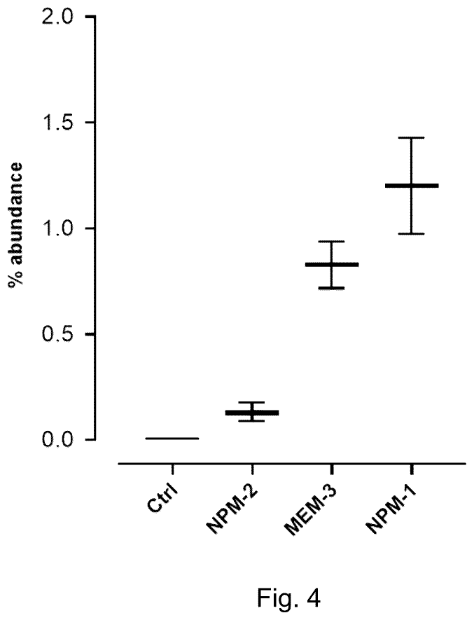

Further, the inventor has surprisingly found that an extract comprising at least two SMs that are encapsulated with an acid resistant coating leads to a significant growth of beneficial microorganisms in the gut and/or a significant reduction in the abundance of bacteria associated with negative health impacts, as shown in FIGS. 1-5.

The inventor has surprisingly observed that the beneficial effects could not be obtained when a composition comprising a single SM was administered to a subject, and likewise, when two separate compositions comprising single, but different, SMs were administered to a subject, some beneficial effect was observed, but the best effect was obtained when a single composition comprising two or more SMs encapsulated in an acid resistant coating was administered to a subject. Without being bound by theory, the inventor believes that a synergistic effect is obtained since the encapsulation in the acid resistant coating provides that the two or more SMs are supplied simultaneously to the target bacterium.

The synergistic effect is particularly pronounced when the two or more SMs are from different plants. In some instances, a SM from a different plant may be produced recombinantly in yeast, yet still have the same beneficial effect as provided by the original plant extract or a concentrate thereof.

In an embodiment, the two or more SMs are from different plants. In one embodiment, a composition as disclosed herein comprises at least one of said two or more extracts in said mixture of two or more extracts which is a plant extract. In one embodiment, a composition as disclosed herein comprises at least two extracts of said mixture of two or more extracts which are plant extracts. In one embodiment, a composition as disclosed herein comprises at least two extracts of said mixture of two or more extracts are plant extracts, wherein all extracts of said mixture of two or more extracts are plant extracts.

Specifically, the extract may be obtained from plant material from different plants, or one or all of the two or more SMs may be obtained from genetically modified microorganisms or synthesized. Such a composition has the benefit of having diverse SMs, which may have a synergistic effect on the composition of microorganisms in the gut. In some embodiments plants are preferably selected from Adansonia, Allium sativum, Avena sativa L, Bacopa monnieri, Boswellia serrata, Camellia sinensis, Centella asiatica, Chicorium intybus, cholecalciferol, Citrus limon, Citrus paradisi, Citrus sinensis, Curcuma longa, cyanocobalamin, Fagopyrum esculentum, Gingko biloba, GGriffonia simplicifolia, Hericium erinaceus, Hordeum vulgare, Humulus lupulus, Hypericum perforatum, Malpighia glabra, Melissa officinalis, Momordica charantia, Mormodica charantia, Panax ginseng, Panax ginseng, Passiflora incarnata, Piper nigrum, Prunus cerasus, pyridoxine, Rhodiola rosea, Riboflavin, Rosa canina, Salvia officinalis L, Salvia officinalis L, Taraxacum officinale, Theobroma cacao, Trigonella foenum-graecum, Tumeric rhizome, Vaccinium myrtillus, Valeriana officinalis, Vitis vinifera, Withania somnifera, Zingiber officinale and Ziziphus jujuba. The plants are preferably selected from Bacopa monnieri, Gingko biloba, Boswellia serrata, Panax ginseng, Vitis vinifera, Vaccinium myrtillus, Ziphus jujube, Centella asiatica, Hericium erinaceus, Rhodiola rosea, Withania somnifera, Camellia sinensis, and Curcuma longa.

In one embodiment, a composition comprises at least one of said two or more SMs in said mixture of two or more extracts, which is provided by a plant, yeast or fungi source different from at least one other SM, i.e. at least two SMs are derived from two different sources. In one embodiment, a composition as disclosed herein comprises one or more of said SMs which have been derived by recombinant production in yeast. In one embodiment, a composition as disclosed herein comprises at least one of said two or more extracts in said mixture of two or more extracts, which is a plant extract from which at least one of said one or more secondary metabolites is provided and wherein another of said one or more SMs in said mixture of two or more extracts is a SM derived from a yeast source by production of a SM in yeast.

One aspect of the present disclosure relates to the use of an extract containing two or more SMs, wherein the SMs constitute at least 10% (w/w) by dry weight of the extract, which SMs comprised in a composition resistant to dissolution or disintegration at pH below 4, preferably pH about 4.8, as a prebiotic in a vertebrate animal, e.g., a mammal, e.g., in the manufacture of a prebiotic for a vertebrate animal, e.g., a mammal. One embodiment of the present disclosure relates to the use of an extract containing two or more SMs, wherein the SMs constitute at least 10% (w/w) by dry weight of the extract, which SMs are encapsulated in an acid resistant coating, as a prebiotic in a vertebrate animal, e.g., a mammal, e.g., in the manufacture of a prebiotic for a vertebrate animal, e.g., a mammal. In one embodiment said acid resistant coating is resistant to dissolution or disintegration at pH below 4, preferably below pH about 4.8.

The composition comprises an extract containing SMs. However, the SMs may be obtained in any way, and the SMs need not be provided from plants. For example, the SM may be provided from any organism or biological material containing the relevant SMs. For instance, the extract may be of a genetically modified microorganism, e.g., a yeast, which has been engineered to produce the SMs. The extract may also be of a fungus. Correspondingly, the SMs may also be synthesized chemically. The extract may also be of a plant, a plant part, a plant-derived material, or a combination thereof. For instance, the extract may be of one or more of flowers, a fruit, a root, a nut, or a berry, leaves, stems, tubers, etc., or any mixtures of these.

The extract may be obtained using any procedure as desired, and the extract may have a solvent content, or the extract may be in a dry form. The extract contains two or more SMs, which constitute at least 10% (w/w) by dry weight of the extract. Each of the two or more SMs may be contained in any proportion in the extract, as long as the total amount of the SMs constitutes at least 10% (w/w) by dry weight of the extract. In the context of the invention, the extract is obtained from a “base material”. In general, the extraction involves separation of the SMs from other constituents in the base material, and thereby the SM will typically be enriched, and to some degree also purified, compared to the SM as it is contained in the base material. However, it is also contemplated that a base material containing a plant secondary metabolite, e.g., a first and/or a second plant secondary metabolite, in a sufficiently high amount, i.e., in the dry weight of the base material, may be used directly as the extract, or that a base material containing a first SM may be mixed together with a further extract containing a second plant secondary metabolite.

In some embodiment the base material has been enriched by at least a factor of 2, which means that the content of SM in e.g., the plant extract is 2 times (double) of the amount in the dry natural (unmodified) material, this factor is hereinafter denominated “enrichment factor”. Herein “unmodified” referring to natural material or extracts, means that nothing has been performed on the natural occurring plant material, other than drying of the plant material selected. In some embodiment the base material has been enriched by at least a factor of 3, which means that the content of SM in the e.g., plant extract is 3 times (triple) of the amount in the dry natural (unmodified) material. In some embodiments the enrichment factor of the extract used is at least 3. In some embodiments the enrichment factor of the extract used is at least 4. In some embodiments the enrichment factor of the extract used is at least 5. In some embodiments the enrichment factor of the extract used is 6. In some embodiments the enrichment factor of the extract used is at least 7. In some embodiments the enrichment factor of the extract used is at least 4. In some embodiments the enrichment factor of the extract used is at least 8. In some embodiments the enrichment factor is above 10, 20, 30 or more.

In some embodiments the base material comprises at least one SM selected from the group consisting of alkaloids, amino acids, curcuminoids, glycosides, polyphenols, polysaccharides, terpenes, and vitamins, optionally at least one plant secondary metabolite is not a polysaccharide. In some embodiments said at least one SM constitutes at least 0.3-2% (w/w) of said base material. In some embodiments said at least one SM constitutes at least 2-5% (w/w) of said base material. In some embodiments said at least one SM constitutes at least 5-10% (w/w) of said base material. In some embodiments said at least one SM constitutes at least 10-15% (w/w) of said base material. In some embodiments said at least one SM constitutes at least 15-20% (w/w) of said base material. In some embodiments said at least one SM constitutes at least 20-30% (w/w) of said base material.

In some embodiments said at least one SM constitutes at least 30-50% (w/w) of said base material. In some embodiments said at least one SM constitutes at least 50-70% (w/w) of said base material. In some embodiments said at least one SM constitutes at least 70-100% (w/w) of said base material. In some embodiments the base material is 95-100% (w/w) SM, such as e.g., in vitamin powders or similar.

When the base material is a plant or a part of a plant, or when the base material is a microorganism, e.g., genetically modified microorganism, the base material may be extracted with a solvent. Any solvent may be used, but in general the solvent will be selected based on the type of plant secondary metabolite, e.g., based on the acidity, basicity, polarity, hydrophobicity, etc. of the plant secondary metabolite. Examples of solvents that may be used are water, alcohols, e.g., methanol, ethanol, propanol, etc., aldehydes, ketones, e.g., acetone, alkanes, e.g., hexane, chloroform, ethers, esters, e.g., ethyl acetate, or mixtures of solvents, such as 50 to 70% ethanol in water. The solvent may also be a supercritical solvent, e.g., supercritical CO₂, supercritical ethanol, or supercritical water. The base material may, before being extracted, be processed into smaller portions such as by grinding. After extraction of the base material with a solvent, the extract (solvent containing extracted matter) is removed from the base material, such as by filtering or sieving.

In an embodiment, the extract is in a dry form. For example, the extract may be provided from extraction of the base material, e.g., a plant material, using an extract, e.g., a polar solvent, such as water or a mixture of ethanol and water, followed by removal of the solvent. e.g., by evaporation, distillation, e.g., vacuum distillation. It is generally preferred that the solvent is removed at low temperature to stabilise the plant secondary metabolite. The extract, especially an extract in dry form, may comprise any auxiliaries. Appropriate auxiliaries comprise granulation agents, binding agents, and stabilisers, such as maltodextrin and/or silicon dioxide. When the extract is in a dry form, it is preferred that it is in a particulate form, e.g., as a granulate, having particles in the size range of 0.1 mm to 3 mm. For example, >95% of the particles may make it through an 80 mesh and be of for instance on average 180 μm in size or 177 μm in size, or more than 98% may pass an 80 mesh. The particle size may be measured by the Active Standard ASTM E1037.

When the composition has a solvent content, the solvent may originate from the extraction or be an additive to the extract or be a slight water content due to a hygroscopic nature of the extract. In a preferred embodiment, the extract is a powder or granulates having low to no solvent content. Generally, when the composition contains organic solvents, the compositions may comply with the United States Pharmacopeia and/or the European pharmacopoeia. When the composition contains the solvent methanol, the composition may contain less than 3000 ppm methanol, and/or when the composition contains the solvent ethanol, the composition may contain less than 5000 ppm ethanol.

In one embodiment, a composition as disclosed herein is resistant to dissolution below pH about 4, preferably pH about 4.8 said composition comprising an extract and optionally one or more pharmaceutically or nutritionally acceptable excipients, wherein said extract is a mixture of several extracts, preferably a mixture of two or more extracts and wherein said mixture of two or more extracts comprises at least two SMs. In one embodiment if one of said at least two secondary metabolites is a polysaccharide, then said other secondary metabolite is selected from the group consisting of: alkaloids, amino acids, curcuminoids, glycosides, polyphenols, polysaccharides, terpenes and vitamins.

In one embodiment if one of said at least two secondary metabolites is a polysaccharide, then said other secondary metabolite is selected from the group consisting of: polyphenols, curcuminoids, alkaloids, terpenes and glycosides.

In one embodiment if one of said at least two secondary metabolites is a polysaccharide, then said another secondary metabolite is not a polysaccharide. In one embodiment if one of said at least two SMs is a triterpenoid, then said second SM is not oxalic acid. In one embodiment, if one SM is a curcuminoid, then a second SM is not huperzine A.

In one embodiment if one of said at least two SM is a polyphenol, such as a flavon, then at least one other of said at least two SM has to at least be a different SM from the polyphenol type, e.g., anthocyanidins, gingerol, flavonoids, flavonoids, flavanones, flavones. In one embodiment if one of said at least two secondary metabolites is a polyphenol, such as anthocyanidin then at least one other of said at least two SM has to at least be a different SM from the anthocyanidin type, e.g., polyphenol, gingerol, flavonoids, flavonoids, flavanones, flavones. Two or more SMs may be of the same class A, or they may be of different classes A. Two or more SMs may be of the same class A and they may be of different classes B. In an embodiment, the composition comprises more than two SMs, e.g., the composition comprises 3, 4, 5, 6, 7, 8, 9 or 10, or more than 10 SMs, e.g., SMs of two classes, three classes or all four classes. In some embodiments the composition comprises 10, 11, 12, 13, 14 or 15 SMs.

In general, the two or more SMs constitute at least 10% (w/w) by dry weight of the extract. In some embodiments the two or more SMs constitute at least 20% (w/w) by dry weight of the extract. In some embodiments the two or more SMs constitute at least 30% (w/w) by dry weight of the extract. In some embodiments, when the composition comprises more than two SMs, each SMs should constitute at least about 1% (w/w), preferably 3% (w/w), e.g., at least 4% (w/w), or at least 5% (w/w). However, in particular when the composition comprises more than two SMs, at least one of said SM should constitute at least 3% (w/w), e.g., at least 4% (w/w), or at least 5% (w/w). In particular embodiments when the composition comprises more than two SMs, each of said SM may constitute at least 3% (w/w), e.g., at least 4% (w/w), or at least 5% (w/w). In some embodiments a least two of said SMs constitute at least 3% (w/w), e.g., at least 4% (w/w), or at least 5% (w/w). A single SM may also be present in at least 10% (w/w) of the extract, and in this case the second SM should be present in at least 2% (w/w) of the extract.

In an embodiment, the SMs comprise polyphenols and terpenes, such as triterpenoids. Suitably, terpenes, such as triterpenoids constitute at least 1% (w/w) by dry weight of the extract and polyphenols constitute at least 7.5% (w/w) by dry weight of the extract. In an embodiment, the SMs comprise glycosides and polyphenols. In an embodiment, the SMs comprise terpenes and polyphenols. In an embodiment, the SMs comprise terpenes, such as triterpenoids and polyphenols. Suitably, triterpenoids constitute at least 1% (w/w) by dry weight of the extract and polyphenols constitute at least 7.5% (w/w) by dry weight of the extract.

In an embodiment, the SMs comprise triterpenoids and polyphenols, such as flavones. Suitably, triterpenoids constitute at least 0.1-32.5% (w/w) by dry weight of the extract, polyphenols constitute at least 0.6-60% (w/w) by dry weight of the extract, and further polyphenols specifically selected as flavones constitute at least 3.5% (w/w) by dry weight of the extract. Suitably, triterpenoids constitute at least 0.5% (w/w) by dry weight of the extract, polyphenols constitute at least 8-24% (w/w) by dry weight of the extract, and flavones constitute at least 6% (w/w) by dry weight of the extract.

In an embodiment, the SMs comprise triterpenoids and polyphenols, such as flavones. Suitably, triterpenoids constitute at least 0.5% (w/w) by dry weight of the extract, polyphenols constitute at least 18% (w/w) by dry weight of the extract, and further polyphenols specifically selected as flavones constitute at least 6% (w/w) by dry weight of the extract. Suitably, triterpenoids constitute at least 0.5% (w/w) by dry weight of the extract, polyphenols constitute at least 18% (w/w) by dry weight of the extract, and flavones constitute at least 6% (w/w) by dry weight of the extract.

In an embodiment, the SMs comprise triterpenoids, polyphenols, and flavones, the triterpenoids constituting at least 30% (w/w) by dry weight of the extract, the polyphenols constituting at least 7% (w/w) by dry weight of the extract, and the flavones constituting at least 6% (w/w) by dry weight of the extract. In an embodiment, the nutraceutical composition comprises extracts of Gingko biloba, Centella asiatic, Panax ginseng, and Hericium erinaceus and the SMs comprise triterpenoids, polyphenols, and flavones, the triterpenoids constituting at least 30% (w/w) by dry weight of the extract, polyphenols constituting at least 7% (w/w) by dry weight of the extract, and flavones constituting at least 6% (w/w) by dry weight of the extract.

In an embodiment, the SMs comprise terpenes, such as triterpenoids and polyphenols, the terpenes, such as triterpenoids constituting at least 10% (w/w) by dry weight of the extract and the polyphenols constituting at least 40% (w/w) by dry weight of the extract. In an embodiment, the SMs comprise triterpenoids and polyphenols, the triterpenoids constituting at least 10% (w/w) by dry weight of the extract and the polyphenols constituting at least 40% (w/w) by dry weight of the extract.

In an embodiment, the nutraceutical composition comprises extracts of Bacopa monnieri, Gingko biloba, Boswellia serrata, Vaccinium myrtillus, Vitis vinifera, and Hordeum vulgare. In an embodiment, the SMs comprise terpenes, such as triterpenoids, polyphenols, polysaccharides, such as beta glucan, and additional polyphenols, such as flavones. In an embodiment, the SMs comprise triterpenoids, polyphenols, beta glucan, and flavones. In an embodiment, the SMs comprise triterpenoids and flavones. Suitably, triterpenoids constitute at least 16% (w/w) by dry weight of the extract and flavones constitute at least 6% (w/w) by dry weight of the extract.

In an embodiment, the nutraceutical composition comprises extracts of Withania somnifera, Bacopa monnieri, Gingko biloba, and Boswellia serrata and the SMs comprise triterpenoids and flavones, the triterpenoids constituting at least 16% (w/w) by dry weight of the extract and the flavones constituting at least 6% (w/w) by dry weight of the extract. In some embodiments, the SMs constitute at least 15, at least 20, at least 25, at least 30, at least 35, at least 40, at least 45 or at least 50% (w/w) by dry weight of the extract. In an embodiment, the acid resistant coating dissolves at a pH-value of above pH 4, such as pH 4.5 or ph 4.8.

The acid resistant coating may be any enteric coating. Enteric coatings are well-known to the skilled person. The acid resistant coating may be of a plant-based material, such as cellulose acetate. The acid resistant coating may be a synthetic material. The acid resistant coating may be hypromellose, pullulan, cellulosic derivatives, modified starch, copolymers based on methacrylic acid and ethyl acrylate, copolymer based on methyl acrylate, methyl methacrylate and methacrylic acid, cellulose acetate phthalate, cellulose acetate succinate, hydroxypropyl methyl cellulose phthalate, hydroxypropyl methyl cellulose acetate succinate (hypromellose acetate succinate), polyvinyl acetate phthalate (PVAP), shellac, cellulose acetate trimellitate, or sodium alginate.

In some embodiments said plant secondary metabolites or said composition is encapsulated in an acid resistant coating and wherein said encapsulation is a microencapsulation or a nanoencapsulation or wherein said extract comprising several extracts is provided in a capsule resistant to dissolution in the stomach acid.

In an embodiment, the composition further comprises a non-digestible plant fibre comprising polysaccharides, oligosaccharides, lignins or their mixtures, which non-digestible plant fibre is encapsulated in the acid resistant coating. Such fibers may preferable be known pre-biotic fibers such as fructan oligosaccharide, araban oligosaccharide, inulin, lactulose, cellulose, chitin, hemicellulose, hexoses, pentose, xanthan gum, resistant starch, arabinoxylan, fructans, inulin, polyuronide, pectin, alginic acids (alginates) such as sodium alginate, potassium alginate, ammonium alginate, calcium alginate, propylene glycol alginate (PGA), and carrageen, raffinose, and rolydextrose. In a preferred embodiment, the non-digestible plant fibre is a beta glucan or a xylooligosaccharides. The non-digestible plant fibre (polysaccharides) is an energy source for the microorganisms of the gut. Without being bound by theory it is believed that this energy source further increases the growth of beneficial bacteria in the gut, when combined with the two or more SMs of the nutraceutical composition. In a sense, the non-digestible plant fibre can be considered an activator of growth, whereas the SMs can be considered a modifier, which increases the grow of certain bacteria. Thereby, a composition is provided, which has the synergistic effect of the two or more SMs on the target bacteria and at the same time provides growth activation. Therefore, the non-digestible plant fibre may be selected based on the genus or species of beneficial intestinal bacteria and/or the genus or a species of intestinal bacteria with a negative impact on health.

In an embodiment, the two or more SMs are selected from a phenol, a polyphenol, a flavonoid, an anthocyanin, a terpenoid, a terpene, a saponin, an alkaloid, a lactone, a glycoside, or their mixtures. In an embodiment, the SMs comprise a phenol, a polyphenol, a flavonoid, an anthocyanin, or their mixtures and a terpenoid or a terpene or their mixtures.

The inventor has found that such a combination of SMs results in a suitable increase in abundance of Bifidobacteria and/or Faecalibacteria and/or Bacteroides and/or Prevotella and/or a suitable decrease in the abundance of Blautia. The inventor has further found that such SMs combinations may be useful in decrease the same, if the bacteria genus abundancy is not balancing, e.g., to high.

In some embodiments, the SMs are encapsulated by microencapsulation or nanoencapsulation. The SMs may for instance be microencapsulated or nanoencapsulated by ethyl cellulose, polyvinyl alcohol, gelatine or sodium alginate. The microencapsulation or nanoencapsulation may be achieved by any suitable techniques, such as pan coating, centrifugal extrusion, vibrational nozzle, spray-drying, ionotropic gelation, coacervation-phase separation, interfacial polycondensation, interfacial cross-linking, in situ polymerization, matrix polymerisation. When microencapsulation or nanoencapsulation are employed, it is preferred that the extract is in a dry, particulate form with particles in the size range of 0.1 mm to 1 mm, e.g., 0.2 mm to 0.5 mm. By microencapsulating or nanoencapsulating the plant secondary metabolites, the nutraceutical composition may take the form of a powder-like substance, such as known from protein powder or flour. Advantageously, the microencapsulated nutraceutical composition, especially in the form of a powder-like substance, may be consumed as an additive in a regular meal and may be chewed without severely breaking the encapsulation. For example, the microencapsulated nutraceutical composition, e.g., in the form of a powder-like substance, may be mixed with other food ingredients to provide a food product in a desirable form, e.g., a food bar or the like. Other encapsulations, such as a common capsule, may for instance break if chewed, and lead to a leakage of material into the stomach acid. Being able to add the nutraceutical composition to a meal and being able to chew the meal without breaking the microencapsulation or nanoencapsulation would mean that the consumption of the nutraceutical composition would be more palatable for the mammal or person while still retaining the effect of the acid resistant encapsulation/coating.

In an embodiment, the two or more SMs are obtainable from a plant selected from the list consisting of: Bacopa monnieri, Gingko biloba, Boswellia serrata, Panax ginseng, Vitis vinifera, Vaccinium myrtillus, Ziziphus jujube, Centella asiatica, Hericium erinaceus, Rhodiola rosea, Withania somnifera, Camellia sinensis, and Curcuma longa. The inventor has found that SMs of these plants have a suitable effect on the growth of Bifidobacteria and/or Faecalibacteria and/or Bacteroides and/or Prevotella in the gut and/or a suitable decrease in the abundance of Blautia.

In one embodiment the bacteria genus to increase or decrease by the extract comprising at least two plant secondary metabolites as disclosed herein can be selected from the group consisting of: bifidobacterium, faecalibacterium, sutterella, bacteroides, rosburia, eubacterium, blautia, prevotella, dorea, ruminococcus, escherichia, enterobacter, coprococcus, veillonella, lactobacillus, streptococcus, bacillus, clostridium, salmonella, corynebacterium, holdemania, akkermansia, slackia, collinsella, methanobacterium, parabacteroides and christensenella. In one embodiment the bacteria genus to increase or decrease by the extract comprising at least two plant secondary metabolites as disclosed herein can be selected from the group consisting of: bifidobacterium, faecalibacterium, sutterella, bacteroides, rosburia, eubacterium, blautia, prevotella, dorea, ruminococcus, escherichia, enterobacter, coprococcus, veillonella, lactobacillus, streptococcus, bacillus, clostridium, salmonella, corynebacterium, holdemania, akkermansia, slackia, collinsella, parabacteroides and christensenella. In one embodiment the bacteria genus to increase or decrease by the extract comprising at least two plant secondary metabolites as disclosed herein can be selected from the group consisting of: bifidobacterium, faecalibacterium, sutterella, bacteroides, rosburia, eubacterium, blautia, prevotella, dorea, ruminococcus, escherichia, enterobacter, coprococcus, veillonella, lactobacillus, streptococcus, bacillus, clostridium, salmonella, corynebacterium, holdemania, akkermansia, slackia, collinsella, parabacteroides and christensenella.

In one embodiment the bacteria genus to increase or decrease by the extract comprising at least two plant secondary metabolites as disclosed herein can be selected from the group consisting of: akkermansia, bacteroides, bifidobacterium, clostridium, coprococcus, eubacterium, escherichia, faecalibacterium, lactobacillus, roseburia, streptococcus and ruminococcus. In one embodiment the bacteria genus to increase by the extract comprising at least two plant secondary metabolites as disclosed herein can be selected from the group consisting of: bacteroides, bifidobacterium, clostridium, coprococcus, eubacterium, escherichia, faecalibacterium, lactobacillus, roseburia and streptococcus. In one embodiment the bacteria genus to decrease by the extract comprising at least two plant secondary metabolites as disclosed herein can be selected from the group consisting of: akkermansia, bacteroides, bifidobacterium, clostridium, coprococcus, eubacterium, faecalibacterium, lactobacillus, roseburia and ruminococcus.

When orally administering the composition of the present disclosure, the dose per day may preferably be in the range of 0.4 to 1.6 grams of composition (excluding the weight of the acid resistant coating). This corresponds to the content of 1 to 4 capsules size 0 per day or the content of 1 to 3 capsules size 00 per day.

In an embodiment, the composition of the present disclosure is for use in therapy. In an embodiment, the composition of the present disclosure is for use in the treatment of obesity. Obesity may be described in terms of the body mass index (BMI), which is defined as the body mass divided by the square of the body height, e.g., as expressed in units of kg/m². In an embodiment, the composition of the present disclosure is for use in the treatment of type 2 diabetes. In an embodiment, the composition of the present disclosure is for use in the treatment or prevention of obesity. In an embodiment, the composition of the present disclosure is for use in regulating appetite. In an embodiment, the composition of the present disclosure is for use in increase of mental focus. In an embodiment, the composition of the present disclosure is for use in reducing stress and/or anxiety. In an embodiment, the composition of the present disclosure is for use in regulating appetite. In an embodiment, the composition of the present disclosure is for preventing infections with bacteria and virus, such as influenza rhino viruses.

In an embodiment, the composition of the present disclosure is for use in the treatment of colon-related dysfunction. In an embodiment, the composition of the present disclosure is for use in the treatment of cognitive dysfunction, such as dementia, such as Alzheimer's disease.

In an embodiment, the composition of the present disclosure is for use in the treatment of attention deficit hyperactivity syndrome (ADHD). In an embodiment, the composition of the present disclosure is for use in the treatment of depression, anxiety, or other stress-related mental dysfunction(s).

In one embodiment, a composition as disclosed herein comprises at least one of said two or more SMs constituting at least about 1% (w/w) to at about 3% (w/w) by dry weight of the extract mixture of two or more extracts, said extracts are provided by a plant, yeast or fungi source different from at least one other SMs comprised in said extract, wherein said other SMs constituting at least 1% (w/w) to at about 3% (w/w) by dry weight of the extract mixture of two or more extracts.

In one embodiment, a composition as disclosed herein comprises an extract comprising at least two SMs constituting of about 1% (w/w) or more by dry weight of said extract, wherein each SM is provided to the extract by two plant extracts.

In one embodiment, a composition as disclosed herein comprises an extract comprising at least two SMs constituting of about 1% (w/w) or more by dry weight of said extract, wherein each SM is provided to the extract by two or more plant extracts. In one embodiment, a composition as disclosed herein comprises an extract comprising at least two SMs constituting of about 1% (w/w) or more by dry weight of said extract, wherein each SM is provided to the extract by between two and 15 plant extracts.

In one embodiment, a composition as disclosed herein comprises an extract comprising at least two SMs constituting of about 1% (w/w) or more by dry weight of said extract, wherein each SM is provided to the extract by three, four or five plant extracts. In one embodiment, a composition as disclosed herein comprises an extract comprising at least two SMs constituting of about 1% (w/w) or more by dry weight of said extract, wherein each SM is provided to the extract by six, seven or eight plant extracts. In one embodiment, a composition as disclosed herein comprises an extract comprising at least two SMs constituting of about 1% (w/w) or more by dry weight of said extract, wherein each SM is provided to the extract by nine, 10, 11 or 12 plant extracts.

In one embodiment, a composition as disclosed herein comprises an extract comprising at least two SMs, wherein said composition comprises at least about 10% (w/w) by dry weight of said extract, or at least about 9% (w/w) by dry weight of the composition.

In certain embodiments of the present invention, the oral composition comprises excipients commonly found in e.g., neutritional or pharmaceutical compositions, examples of such excipients include, but are not limited to enzyme inhibitors, stabilisers, preservatives, flavours, sweeteners and other components. Other excipients colorants, fillers, binding agents, lubricants, disintegrants, or wetting agents. ingredients Such components are described in e.g., ‘Handbook of Pharmaceutical Excipients’ Ainley Wade, Paul J. Weller, Arthur H. Kibbe, 3rd edition, American Pharmacists Association (2000), which is hereby incorporated by reference or—Handbook of Pharmaceutical Excipients', Rowe et al., Eds., 4th Edition, Pharmaceutical Press (2003), which is hereby incorporated by reference.

In one embodiment a pharmaceutical composition according to the present invention comprises excipients known to the person skilled in the art.

In certain embodiments of the present invention, the oral composition comprises excipients commonly found in e.g., neutritional or pharmaceutical compositions. Such as fillers known to the person skilled in the art. Non-limiting examples of such known excipients are disclosed in “Direct compression and the role of filler-binders” (p 173-217): by B. A. C. Carlin, in “Disintegrants in tabletting” (p 217-251): by R. C. Moreton, and in “Lubricants, glidants and adherents” (p 251-269), by N. A. Armstrong, in Pharmaceutical dosage forms: Tablets”, Informa Healthcare, N.Y., vol 2, 2008, L. L. Augsburger and S. W. Hoag”, and incorporated herein by reference.

In one embodiment, a composition as disclosed herein comprises one or more pharmaceutically or nutritionally acceptable excipients present in about 1% to about 2% (w/w) by dry weight of said composition. In one embodiment, a composition as disclosed herein comprises one or more pharmaceutically or nutritionally acceptable excipients, wherein said excipient is a filler and constitutes about 1% to about 2% (w/w) by dry weight of said composition. In one embodiment, a composition as disclosed herein comprises one or more pharmaceutically or nutritionally acceptable excipients, wherein said excipient is a filler and constitutes about 1% to about 2% (w/w) by dry weight of said composition, wherein said filler is a rice extract or a blend of fillers comprising rice extracts. In one embodiment, a composition as disclosed herein comprises one or more pharmaceutically or nutritionally acceptable excipients, wherein said excipient is a filler and constitutes about 1% to about 2% (w/w) by dry weight of said composition, wherein said filler is a blend of natural ingredients comprising one, more or all ingredients selected from the list consisting of: rice extract, rice hulls, arabic gum and sunflower oil.

In one embodiment, at least two SMs are selected from the group consisting of alkaloids, amino acids, curcuminoids, glycosides, polyphenols, polysaccharides, terpenes and vitamins. In one embodiment, at least two SMs are selected from the group consisting of alkaloids, curcuminoids, glycosides, polyphenols, polysaccharides and terpenes. In one embodiment, said SMs constitute at least about 10% to about 85% (w/w) by dry weight of the extract. In one embodiment, said SMs constitute up to about 85% (w/w) by dry weight of the extract. In one embodiment, said SMs constitute at least about 15% to 60% (w/w) by dry weight of the extract, said SMs constitute at about 10% (w/w) by dry weight of the extract or at about 9% (w/w) by dry weight of said composition. In one embodiment, said SMs constitute at about 18-19% (w/w) by dry weight of the extract. In one embodiment, said SMs constitute at about 20-21% (w/w) by dry weight of the extract. In one embodiment, said SMs constitute at about 23-24% (w/w) by dry weight of the. In one embodiment, said SMs constitute at about 27-28% (w/w) by dry weight of the extract. In one embodiment, said SMs constitute at about 54-55% (w/w) by dry weight of the extract. In one embodiment the extract comprises at least 10% (w/w) SMs by dry weight of the extract selected from the group consisting of: alkaloids, curcuminoids, glycosides, polyphenols, polysaccharides and terpenes.

In one embodiment, the composition comprises at least two SMs, wherein the total concentration of SMs in said extract, selected from the group of SMs consisting of: alkaloids, amino acids, curcuminoids, glycosides, polyphenols, polysaccharides, terpenes and vitamins is at least 10%.

In one embodiment said at least two SMs are polyphenols. In some embodiments polyphenols may be selected from the list consisting of: anthocyanidins, anthocyanin, apigenin flavone glycoside, flavonoid, flavones, flavonoids, gingerol, phenolic acids, rosmarinic acid, hydroxycinnamic acid and vexitin. In one embodiment the extract does not comprise SMs from the category of amino acids. In one embodiment the extract does not comprise SMs from the categories amino acids and vitamins. In one embodiment the extract does comprise between about 0 to about 30% (w/w) amino acids. In one embodiment the extract does comprise between about 0 to about 10% (w/w) amino acids. In one embodiment the extract does comprise between about 5 to about 20% (w/w) amino acids. In one embodiment an extract comprises between about 0% (w/w) to about 50% (w/w) SMs which are amino acids. In one embodiment said extract comprises between about 0% (w/w) to about 50% (w/w) SMs which are amino acids. In one embodiment said extract comprises between about 0% (w/w) to about 30% (w/w) SMs which are amino acids. In one embodiment said extract comprises between about 0% (w/w) to about 20% (w/w) SMs which are amino acids. In one aspect said extract comprises between about 0% (w/w) to about 10% (w/w) SMs which are amino acids.

In one embodiment said at least two SMs are polyphenols, constituting at between about 0.5% to 50% (w/w) or more by dry weight of said extract. In one embodiment said at least two SMs are polyphenols, constituting at between about 5% to 25% (w/w) or more by dry weight of said extract. In one embodiment said at least two SMs are polyphenol, constituting at between about 5% to about 25% (w/w) or more by dry weight of said extract. In one embodiment said at least two SMs are polyphenol, constituting at least about 1% (w/w) or more by dry weight of said extract, or at least about 0.5% (w/w) or more by dry weight of said composition. In one embodiment said at least two SMs are polyphenol, constituting at least about 3% (w/w) or more by dry weight of said extract, or at least about 2.5% (w/w) or more by dry weight of said composition. In one embodiment said at least two SMs are polyphenols, constituting about 5% (w/w) by dry weight of the extract. In one embodiment said at least two SMs are polyphenols, constituting at between about 8% (w/w) by dry weight of the extract. In one embodiment said at least two SMs are polyphenols, constituting at between about 16% (w/w) by dry weight of the extract. In one embodiment said at least two SMs are polyphenols, constituting at between about 23% (w/w) by dry weight of the extract.

In one embodiment said at least two SMs are glycosides. In some embodiments glucosides may be selected independently from the group consisting of: flavonoid glycosides, ginsenosides, rosavins, saponins, triterpene glycosides and triterpenoid saponins. In some embodiments glycosides include terpenes, such as triterpenoids and thus glycosides and may independently be selected from the group consisting of: flavonoid glycosides, ginsenosides, rosavins, saponins, triterpene glycosides, triterpenoid saponins. In some embodiments terpenes are selected from the group consisting of: terpenoids, such as boswellic acids; valerenic acid; α-pinene; triterpene glycosides, such as saponins; steroids, such as triterpenoid withanolides; triterpenoids, such as triterpenoid glycosides.

In one embodiment said at least two SMs are terpenes, constituting at between about 0.1% to about 35% (w/w), such as between 0.1-5.5% (w/w), 5.5-12% (w/w) or 12-25% (w/w) or more by dry weight of said extract. In one embodiment said at least two SMs are terpenes, constituting at between about 0.1% to about 5.5% (w/w) or more by dry weight of said extract. In one embodiment said at least two SMs are terpenes, constituting at between about 15% to about 25% (w/w), such as 16% (w/w), 19% (w/w) or more by dry weight of said extract.

In one embodiment said at least two SMs are glycosides, constituting at between about 0.4% to about 45% (w/w) or more by dry weight of said extract. In one embodiment said at least two SMs are glycoside, constituting at between about 1% to about 45% (w/w) or more by dry weight of said extract. In one embodiment said at least two SMs are glycoside, constituting at between about 2% to about 45% (w/w) or more by dry weight of said extract. In one embodiment said at least two SMs are glycoside, constituting at between about 1% to about 9% (w/w) or more by dry weight of said extract. In one embodiment said at least two SMs are glycoside, constituting about 1% to about 9% preferably, about 1%, about 3% or about 9% (w/w) by dry weight of said extract. In one embodiment said at least two SMs are glycoside, constituting at between about 10% to about 20% (w/w) or more by dry weight of said extract. In one embodiment said at least two SMs are glycoside, constituting at between about 20% to about 45% (w/w) or more by dry weight of said extract. In one embodiment said at least two SMs are glycoside, constituting at between about 0.6% to about 12.5% (w/w) or more by dry weight of said extract. In one embodiment said at least two SMs are glycoside, constituting about 0.5-1% (w/w) or more by dry weight of said extract. In one embodiment said at least two SMs are glycoside, constituting about 2.5% (w/w) or more by dry weight of said extract. In one embodiment said at least two SMs are glycoside, constituting about 12.5% (w/w) or more by dry weight of said extract.

In one embodiment said at least two SMs are glycosides and another of said at least two SMs are polyphenols.

In one embodiment said at least two SMs are glycosides and another of said at least two SMs are polyphenols and another class of plant secondary metabolite is from the class of terpenes. In one embodiment said at least two SMs are glycosides constituting a percentage by dry weight of said extract or composition according to any one of the embodiments described herein and another of said at least two SMs are polyphenols, constituting a percentage by dry weight of said extract or composition according to any one of the embodiments described herein. In one embodiment, the composition comprises at least two SMs, wherein said polyphenols constitute at least about 23% (w/w) by dry weight of said extract and said glycosides constitute at least about 1% (w/w) by dry weight of said extract and optionally other SMs, such as terpenes including triterpenoids constitute below 1% (w/w) each by dry weight of said extract. In one embodiment, the composition comprises at least two SMs, wherein said polyphenols constitute at least about 16% (w/w) by dry weight of said extract and the concentration of glycosides terpenes and triterpenoids in combination constitute at least about 3.5% (w/w) by dry weight of said extract, wherein optionally one or more other SMs, such as polysaccharides may be added to said extract, wherein said optional polysaccharide may constitute at least 6% (w/w) by dry weight of said extract. In one embodiment, the composition comprises at least two SMs, wherein said polyphenols constitute at least 16% (w/w) by dry weight of said extract and said glycosides constitute at least about 12% (w/w) by dry weight of said extract and said composition optionally further comprises SMs selected from the group consisting of polysaccharides and alkaloids, wherein said optional polysaccharides may constitute at least about 21% (w/w) by dry weight of said extract and said optional alkaloids constitute about 5% (w/w) by dry weight of said extract.

In one embodiment said at least two SMs are glycosides and another of said at least two SMs are polyphenols and wherein said composition comprises another SM from the class of polysaccharides. In one embodiment, the composition comprises at least two SMs, wherein said polyphenols constitute at least 16% (w/w) by dry weight of said extract and said glycosides constitute at least about 12% (w/w) by dry weight of said extract and said composition further comprises SMs selected from the group consisting of polysaccharides constituting at least about 21% (w/w) by dry weight of said extract.

In one embodiment said at least two SMs are polysaccharides. In some embodiment, if one of said at least to SMs is a polysaccharide, then at least one other SM is chosen from a class different from polysaccharides, e.g., alkaloids, amino acids, curcuminoids, glycosides, polyphenols, terpenes or vitamins. In some embodiments polysaccharides may be selected from the list consisting of: beta glucan, inulin and pectin.

In one embodiment, the composition comprises at least two SMs, wherein one of said at least two SMs are polysaccharides, constituting at between about 2.5% to about 30% (w/w) or more by dry weight of said extract. In one embodiment said at least two SMs are polysaccharides, constituting at between about 3% to about 30% (w/w) or more by dry weight of said extract. In one embodiment said at least two SMs are polysaccharides, constituting at between about 5% to about 10% (w/w) or more by dry weight of said extract. In one embodiment said at least two SMs are polysaccharides, constituting at between about 10% to about 20% (w/w) or more by dry weight of said extract. In one embodiment said at least two SMs are polysaccharides, constituting at between about 21% to about 30% (w/w) or more by dry weight of said extract. In one embodiment said at least two SMs are polysaccharides, constituting about 2.5% (w/w) or more by dry weight of said extract. In one embodiment said at least two SMs are polysaccharides, constituting about 6% (w/w) or more by dry weight of said extract. In one embodiment said at least two SMs are polysaccharides, constituting about 21% (w/w) or more by dry weight of said extract. In one embodiment said at least two SMs are polysaccharides as described above are combined with polyphenols, glycosides, terpenes and/or alkaloids. In one embodiment said extract comprises at least two SMs, wherein one is a polysaccharide as described above and the other may be selected to be 0-5% (w/w), 5-15% (w/w), 15-25% (w/w) or 5-50% (w/w) polyphenols, 0-5% (w/w), 5-15% or 15-30% (w/w) glycosides and terpenes in combination and/or about 0-5%, 5-25% (w/w) or 25-50% (w/w) alkaloids. In one embodiment said at least two SMs are polysaccharides as described above are combined with 0-5% (w/w) or 5-23% (w/w) polyphenols, 0-5% (w/w) or 5-15% (w/w) glycosides and terpenes in combination and/or about 0-5% (w/w) alkaloids.

In one embodiment said at least two SMs are curcuminoids. In one embodiment curcuminoids are diarylheptanoids. In one embodiment said at least two SMs are curcuminoids and another of said at least two SMs are polyphenols and/or polysaccharides or both. In one embodiment said at least two SMs are curcuminoids and polyphenols. In some embodiments curcuminoids constitute about 3-10% (w/w), 10-15% (w/w), 16-25% (w/w), 26-30% (w/w) or 30-50% (w/w) of dry weight of said extract. In one embodiment said at least two SMs are curcuminoids and polyphenols. In some embodiments curcuminoids constitute about 3-10% (w/w), preferably about 3% (w/w) of dry weight of said extract. In one embodiment said at least two SMs in said extract are a combination of about 3-5% (w/w) curcuminoids and 4-6% (w/w) polyphenols, about 2% (w/w) glycosides, such as terpenes and 10-20% (w/w) vitamins.

In one embodiment said extract comprises at least two SMs, wherein one is a polysaccharide and constitute the percentage by dry weight as recited in any one of the embodiments disclosed herein and glycosides, if present constitute the percentage by dry weight as recited in any one of the embodiments disclosed herein, and polyphenols, if present constitute the percentage by dry weight as recited in any one of the embodiments disclosed herein or if glycosides and polyphenols are present at the same time.

In one embodiment, the composition comprises at least two SMs, wherein a further class of SMs are present in said extract, wherein said further secondary metabolites are selected from the group consisting of curcuminoids, terpenes and alkaloids. In one embodiment, the composition comprises at least two SMs, wherein a further class of SMs are present in said extract and if said further SMs are alkaloids constitute about 0% to about 50% (w/w) by dry weight of said extract, preferably between 1% to about 5% (w/w) by dry weight of said extract or between 23% to about 48% (w/w) by dry weight of said extract.

In one embodiment, the composition comprises at least two SMs, wherein a further class of SMs are present in said extract and if said further SMs are alkaloids constitute about 5% (w/w) by dry weight of said extract.

In one embodiment, the composition comprises at least two SMs, wherein a further class of SMs are present in said extract and if said further SMs are curcuminoids constitute about 0.5% to about 50% (w/w) by dry weight of said extract, preferably between about 1% and 10% (w/w) by dry weight of said extract or between 12% (w/w) and 20% (w/w) or between about 24 to about 50% (w/w) by dry weight of said extract. In one embodiment, the composition comprises at least two SMs, wherein a further class of SMs are present in said extract and if said further SMs are curcuminoids, constitute about 1% (w/w) by dry weight of said extract. In one embodiment, the composition comprises at least two SMs, wherein a further class of SMs are present in said extract and if said further SMs are curcuminoids constitute about 3% (w/w) by dry weight of said extract. In one embodiment, the composition comprises at least two SMs, wherein a further class of SMs are present in said extract and if said further SMs are curcuminoids constitute about 9% (w/w) by dry weight of said extract.

In one embodiment, the composition comprises at least two SMs, wherein a further SM is present in said extract, wherein said further secondary metabolites may a vitamin.

The oral composition, wherein said polyphenol, selected from the class of vitamins constitute at least about 2% (w/w) by dry weight of said extract. The oral composition, wherein said vitamins constitute at about 3% (w/w) by dry weight of said extract. The oral composition, wherein said vitamins constitute about 2-5% (w/w) by dry weight of said extract.

In one embodiment, the composition comprises at least two SMs, said SMs consisting of alkaloids, amino acids, curcuminoids, glycosides, polyphenols, polysaccharides, terpenes and vitamins are independently selected from any one or more of the below listed groups A-I:
A. Alkaloids; selected from the group consisting of: piperine and theobromine;
B. amino acids; selected from the group consisting of: essential amino acids and non-essential amino acids and proteinogenic amino acids also;
C. curcuminoids; selected from the group consisting of: diarylheptanoids;
D. glycosides; selected from the group consisting of: flavonoid glycosides, ginsenosides, rosavins, saponins, triterpene glycosides and triterpenoid saponins;
E. polyphenols; selected from the group consisting of: anthocyanidins, anthocyanin, apigenin flavone glycoside, flavonoid, flavones, flavonoids, gingerol, phenolic acids;
F. rosmarinic acid, hydroxycinnamic acid and vexitin;
G. polysaccharides; selected from the group consisting of: beta glucan, inulin and pectin
H. terpenes; selected from the group consisting of: boswellic acids valerenic acid, α-pinene,
I. vitamins; selected from the group consisting of: vitamin B, C and D

In one embodiment, the composition comprises at least two SMs, wherein said at least one SMs are selected from the group consisting of: rosavins, curcuminoids, saponins, beta glucan, withanolides, flavonoids, amino acids, flavonoids, ginsenosides, flavonoid glycosides, rosmarinic acid, hydroxycinnamic acid, valerenic acid, vitexin, flavanols, flavonoid glycoside, saponins, theobromine, anthocyanidins, gingerol, inulin, piperine, pectin, flavanones, flavones, boswellic acids, flavonoids, glycosides, flavanols and α-pinene.

In one embodiment, the composition comprises at least two SMs, wherein said at least two SMs are selected from the group consisting of: rosavins, curcuminoids, saponins, beta glucan, withanolides, flavonoids, amino acids, flavonoids, ginsenosides, flavonoid glycosides, rosmarinic acid, hydroxycinnamic acid, valerenic acid, vitexin, flavanols, flavonoid glycoside, saponins, theobromine, anthocyanidins, gingerol, inulin, piperine, pectin, flavanones, flavones, boswellic acids, flavonoids, glycosides, flavanols and α-pinene and wherein said SMs are provided by at least two plant extracts, extracted from plants selected from the list consisting of: adansonia, Allium sativum, Avena sativa L, Bacopa monnieri, Boswellia serrata, Camellia sinensis, Centella asiatica, Chicorium intybus, cholecalciferol, Citrus limon, Citrus paradisi, Citrus sinensis, Curcuma longa, cyanocobalamin, Fagopyrum esculentum, Gingko biloba, Griffonia simplicifolia, Hericium erinaceus, Hordeum vulgare, Humulus lupulus, Hypericum perforatum, Malpighia glabra, Melissa officinalis, Momordica charantia, Mormodica charantia, Panax ginseng, Panax ginseng, Passiflora incarnata, Piper nigrum, Prunus cerasus, pyridoxine, Rhodiola rosea, riboflavin, rosa canina, Salvia officinalis L, Salvia officinalis L, Taraxacum officinale, Theobroma cacao, Trigonella foenum-graecum, Tumeric rhizome, Vaccinium myrtillus, Valeriana officinalis, Vitis vinifera, Withania somnifera, Zingiber officinale and Ziziphus jujuba.

In one embodiment, the extract comprises at least about 10-15% (w/w) SMs independently extracted from one or more plants selected from the list consisting of: Piper nigrum, Theobroma cacao, Tumeric rhizome, Rhodiola rosea, Hypericum perforatum, Trigonella foenum-graecum, Mormodica charantia, Panax ginseng, Bacopa monnieri, Passiflora incarnata, Valeriana officinalis, Melissa officinalis, Prunus cerasus, Ziziphus jujuba, Vitis vinifera, Camellia sinensis, Citrus paradisi., Humulus lupulus, Vaccinium myrtillus, Gingko biloba, Citrus sinensis, Zingiber officinale, Avena sativa L, Citrus limon, Chicorium intybus, Salvia officinalis L, Boswellia serrata, Allium sativum, Withania somnifera, Centella asiatica, riboflavin, cholecalciferol, cyanocobalamin, pyridoxine and Malpighia glabra.

In an embodiment, the nutraceutical composition comprises extracts of Rhodiola rosea, Withania somnifera, Centella asiatica, Camellia sinensis, and Curcuma longa.

In an embodiment, the nutraceutical composition comprises an extract of Gingko biloba. In an embodiment, the nutraceutical composition comprises an extract of Centella asiatic. In an embodiment, the nutraceutical composition comprises an extract of Panax ginseng. In an embodiment, the nutraceutical composition comprises an extract of and Hericium erinaceus.

In an embodiment, the nutraceutical composition comprises extracts of Rhodiola rosea, Withania somnifera, Centella asiatica, Camellia sinensis, and Curcuma longa and the SMs comprise glycosides and polyphenols, the glycosides, may be triterpenoids constituting at least 1% (w/w) by dry weight of the extract and the polyphenols constituting at least about 4.5% (w/w) by dry weight of the extract.

In an embodiment, the nutraceutical composition comprises extracts of Rhodiola rosea, Withania somnifera, Centella asiatica, Camellia sinensis, and Curcuma longa and the SMs comprise terpenes and polyphenols, the terpenes constituting at least 1% (w/w) by dry weight of the extract and the polyphenols constituting at least about 4.5% (w/w) by dry weight of the extract.

In an embodiment, the nutraceutical composition comprises extracts of Bacopa monnieri, Gingko biloba, Vitis vinifera, and Camellia sinensis. In an embodiment, the nutraceutical composition comprises extracts of Bacopa monnieri, Gingko biloba, Vitis vinifera, and Camellia sinensis and the SMs comprise glycosides, which may be triterpenoids, polyphenols, and additional polyphenols selected specifically from flavones, wherein the glycosides, such as triterpenoids constituting at least 5% (w/w) by dry weight of the extract, the polyphenols constituting at least 18% (w/w) by dry weight of the extract, and additional phenols, selected as flavones of at least 6% (w/w) by dry weight of the extract.

In an embodiment, the nutraceutical composition comprises extracts of Bacopa monnieri, Gingko biloba, Vitis vinifera, and Camellia sinensis and the SMs comprise glycosides, which may be triterpenoids, polyphenols, and flavones, the glycosides, which may be triterpenoids constituting at least 0.5% (w/w) by dry weight of the extract, the polyphenols constituting at least 18% (w/w) by dry weight of the extract, and flavones of at least 6% (w/w) by dry weight of the extract.

In an embodiment, the nutraceutical composition comprises extracts of Bacopa monnieri, Gingko biloba, Vitis vinifera, and Camellia sinensis and the SMs comprise terpenes, polyphenols, and flavones, the terpenes constituting at least 0.5% (w/w) by dry weight of the extract, the polyphenols constituting at least 18% (w/w) by dry weight of the extract, and flavones of at least 6% (w/w) by dry weight of the extract.

In an embodiment, the nutraceutical composition comprises an extract of Gingko biloba, Centella asiatic, Panax ginseng, and Hericium erinaceus. In an embodiment, the SMs comprise terpenes, such as triterpenoids, polyphenols, and additional phenols selected specifically to be flavones, the glycosides, such as triterpenoids constituting at least 30% (w/w) by dry weight of the extract, the polyphenols constituting at least 7% (w/w) by dry weight of the extract, and the flavones constituting at least 6% (w/w) by dry weight of the extract.

In an embodiment, the nutraceutical composition comprises extracts of Bacopa monnieri, Centella asiatica, Hericium erinaceus, and Vitis vinifera. In an embodiment, the nutraceutical composition comprises extracts of Bacopa monnieri, Gingko biloba, Boswellia serrata, Vaccinium myrtillus, Vitis vinifera, and Hordeum vulgare and the SMs comprise glcosides, which may be triterpenoids, polyphenols, polysaccharides, such as beta glucan, and flavones, the triterpenoids constituting least 5% (w/w) by dry weight of the extracts, the polyphenols constituting at least 8% (w/w) by dry weight of the extracts, the beta glucans constituting at least 1.5% (w/w) by dry weight of the extracts, and the flavones constituting at least 5% (w/w) by dry weight of the extracts.

In an embodiment, the nutraceutical composition comprises extracts of Bacopa monnieri, Gingko biloba, Boswellia serrata, Vaccinium myrtillus, Vitis vinifera, and Hordeum vulgare and the SMs comprise triterpenoids, polyphenols, polysaccharides, such as beta glucan, and flavones, the triterpenoids constituting least 5% (w/w) by dry weight of the extracts, the polyphenols constituting at least 8% (w/w) by dry weight of the extracts, the polysaccharides, such as beta glucans constituting at least 1.5% (w/w) by dry weight of the extracts, and the flavones constituting at least 5% (w/w) by dry weight of the extracts.

In an embodiment, the nutraceutical composition comprises extracts of Bacopa monnieri, Centella asiatica, Hericium erinaceus, and Vitis vinifera and the SMs comprise triterpenoids and polyphenols, the triterpenoids constituting at least 2.5% (w/w) by dry weight of the extract and the polyphenols constituting at least 10% (w/w) by dry weight of the extract.

In an embodiment, the nutraceutical composition comprises extracts of Withania somnifera, Bacopa monnieri, Gingko biloba, and Boswellia serrata.

One embodiment provides for the use of a composition according to any of the embodiments of the invention, in the manufacture of a prebiotic composition for oral administration to a vertebrate animal.

In one embodiment a composition is administered once per day. In one embodiment said composition is administered twice per day. In one embodiment a composition is administered daily, for one month or more. In one embodiment a composition is administered daily for 2, 3, 4 or more months. In one embodiment said composition can be administered daily for a year or more. In one embodiment a composition may be provided in a capsule and the compositions weigh about 300 mg-2000 mg, preferably 400-600 mg each without capsule material. The daily administration of said compositions may be one capsule per day or two capsules per day. In some instances, the compositions may be combined. In some embodiments two or three compositions may be administered at the same time and/or on the same day.

In one embodiment a composition comprises an extract comprising several plant extracts, wherein said plant extract comprises about 24-27% (w/w) plant secondary metabolites, including vitamins and said composition further comprises about 1.5% (w/w) filing agent,

wherein plant extract comprises 26-30% (w/w), preferably about 28% (w/w) or about 90 mg Rhodiola rosea root extract, which is equivalent to about 990 mg dry unmodified Rhodiola rosea root; 22-26% (w/w), preferably about 24% (w/w) or about 95 mg Withania somnifera root extract, which is equivalent to about 760 mg dry unmodified Withania somnifera root;

6-9% (w/w), preferably about 7-8% (w/w) or about 35 mg Hypericum perforatum plant (aerial part) extract, which is equivalent to about 105 mg dry unmodified Hypericum perforatum plant (aerial part); 8-12% (w/w), preferably about 10% (w/w) or about 40 mg Melissa officinalis leaf extract, which is equivalent to about 265 mg dry unmodified Melissa officinalis leaf; 7-11% (w/w), preferably about 9% (w/w) or about 30 mg Valeriana officinalis root extract, which is equivalent to about 225 mg dry unmodified Valeriana officinalis root; 5-9% (w/w), preferably about 7% (w/w) or about 30 mg Passiflora incarnata flower extract, which is equivalent to about 225 mg dry unmodified Passiflora incarnata flower; and 17-21% (w/w), preferably about 19% (w/w) or about 75 mg Prunus cerasus fruit extract, which is equivalent to about 1875 mg dry unmodified Prunus cerasus fruit.

In one embodiment a composition comprises an extract comprising several plant extracts, wherein said plant extract comprises about 24-27% (w/w) plant secondary metabolites, including vitamins and said composition further comprises about 1% (w/w) filing agent,

wherein plant extract comprises 18-22% (w/w), preferably about 20% (w/w) or about 80 mg Rhodiola rosea root extract, which is equivalent to about 720 mg dry unmodified Rhodiola rosea root; 4-8% (w/w), preferably about 6% (w/w) or about 25 mg Vitis vinifera seed extract, which is equivalent to about 625 mg dry unmodified Vitis vinifera seed; 3-7% (w/w), preferably about 5% (w/w) or about 20 mg Ziziphus jujuba fruit extract, which is equivalent to about 200 mg dry Ziziphus jujuba fruit; 17-21% (w/w), preferably about 19% (w/w) or about 75 mg Withania somnifera root extract, which is equivalent to about 600 mg dry unmodified Withania somnifera root; 0-4% (w/w), preferably about 2% (w/w) or about 7.5 mg riboflavin; 0-4% (w/w), preferably about 2% (w/w) or about 38 mg cholecalciferol; 18-22% (w/w), preferably about 18% (w/w) or about 80 mg Avena sativa L extract, which is equivalent to about 720 mg dry unmodified Avena sativa L; 3-8% (w/w), preferably about 5% (w/w) or about 20 mg Salvia officinalis L bran extract, which is equivalent to about 150 mg dry unmodified Salvia officinalis L bran; 8-12% (w/w), preferably about 8% (w/w) or about 40 mg Centella asiatica plant extract, which is equivalent to about 960 mg dry unmodified Centella asiatica plant; and 8-12% (w/w), preferably about 10% (w/w) or about 40 mg Camellia sinensis leaf extract, which is equivalent to about 720 mg dry unmodified Camellia sinensis leaf.

In one embodiment a composition comprises an extract comprising several plant extracts, wherein said plant extract comprises about 24-27% (w/w) plant secondary metabolites, including vitamins and said composition further comprises about 1% (w/w) filing agent, wherein plant extract comprises 17-21% (w/w), preferably about 19% (w/w) or about 75 mg Trigonella foenum-graecum seed extract, which is equivalent to about 938 mg dry unmodified Trigonella foenum-graecum seed; 4-8% (w/w), preferably about 6% (w/w) or about 25 mg Camellia sinensis leaf extract, which is equivalent to about 450 mg dry unmodified Camellia sinensis leaf; 0-3% (w/w), preferably about 0.2% (w/w) or about 1 mg Piper nigrum fruit extract, which is equivalent to about 450 mg dry Piper nigrum fruit; 28-32% (w/w), preferably about 30% (w/w) or about 120 mg Citrus limon skin extract, which is equivalent to about 2400 mg dry unmodified Citrus limon skin; 8-12% (w/w), preferably about 10% (w/w) or about 40 mg Citrus paradisi fruit extract, which is equivalent to about 800 mg dry unmodified citrus fruit paradisi; 17-21% (w/w), preferably about 19% (w/w) or about 75 mg Mormodica charantia fruit extract, which is equivalent to about 300 mg dry unmodified Mormodica charantia fruit; and 13-17% (w/w), preferably about 15% (w/w) or about 60 mg Theobroma cacao bean extract, which is equivalent to about 285 mg dry unmodified Theobroma cacao bean.

In one embodiment a composition comprises an extract comprising several plant extracts, wherein said plant extract comprises about 24-27% (w/w) plant secondary metabolites, including vitamins and said composition further comprises about 1% (w/w) filing agent, wherein plant extract comprises 18-22% (w/w), preferably about 20% (w/w) or about 80 mg Avena sativa L bran extract, which is equivalent to about 720 mg dry unmodified Avena sativa L bran; 18-22% (w/w), preferably about 20% (w/w) or about 80 mg Bacopa monnieri plant extract, which is equivalent to about 520 mg dry unmodified Bacopa monnieri plant; 4-8% (w/w), preferably about 6% (w/w) or about 25 mg Boswellia serrata plant extract, which is equivalent to about 163 mg dry Boswellia serrata perforatum plant; 18-22% (w/w), preferably about 18% (w/w) or about 80 mg Gingko biloba leaf extract, which is equivalent to about 1880 mg dry unmodified Gingko biloba leaf; 8-12% (w/w), preferably about 10% (w/w) or about 40 mg Vaccinium myrtillus fruit extract, which is equivalent to about 1800 mg dry unmodified Vaccinium myrtillus fuit; 0-2% (w/w), preferably about 0.05% (w/w) or about 200 μg cyanocobalamin; 0-4% (w/w), preferably about 2% (w/w) or about 8 mg pyridoxine; and 8-12% (w/w), preferably about 10% (w/w) or about 62.5 mg Panax ginseng root extract, which is equivalent to about 313 mg dry unmodified Panax ginseng root.

In one embodiment a composition comprises an extract comprising several plant extracts, wherein said plant extract comprises about 24-27% (w/w) plant secondary metabolites, including vitamins and said composition further comprises about 1% (w/w) filing agent, wherein plant extract comprises 7-11% (w/w), preferably about 9% (w/w) or about 35 mg Allium sativum bulb extract, which is equivalent to about 350 mg dry unmodified allium sativum bulb; 13-17% (w/w), preferably about 15% (w/w) or about 60 mg Chicorium intybus plant extract, which is equivalent to about 360 mg dry chicorium plant intybus; 6-10% (w/w), preferably about 8% (w/w) or about 33 mg Citrus sinensis skin extract, which is equivalent to about 390 mg dry Citrus sinensis skin; 14-18% (w/w), preferably about 16% (w/w) or about 65 mg Malpighia glabra fruit extract, which is equivalent to about 1365 mg dry unmodified Malpighia glabra fruit; 22-26% (w/w), preferably about 24% (w/w) or about 95 mg Rosa canina fruit extract, which is equivalent to about 380 mg dry unmodified Rosa canina fruit; 8-12% (w/w), preferably about 10% (w/w) or about 40 mg Tumeric rhizome root extract, which is equivalent to about 1200 mg dry unmodified Tumeric rhizome root; 7-11% (w/w), preferably about 9% (w/w) or about 35 mg Vaccinium myrtillus extract, which is equivalent to about 675 mg dry unmodified Vaccinium myrtillus; and 6-10% (w/w), preferably about 8% (w/w) or about 33 mg Zingiber officinale root extract, which is equivalent to about 488 mg dry unmodified zingiber root officinale.

In one embodiment an oral composition is provided which is resistant to dissolution below pH 4, said composition comprising an extract and optionally one or more pharmaceutically or nutritionally acceptable excipients, wherein said extract is a mixture of several extracts, preferably a mixture of two or more extracts and wherein said mixture of two or more extracts comprises at least two plant secondary metabolites.

In one embodiment an oral composition is provided wherein at least one of said two or more plant secondary metabolites in said mixture of two or more extracts, are provided by a plant, yeast or fungi source different from at least one other SM in said extract.

In one embodiment an oral composition is provided wherein two or more or all extracts of said mixture of extracts are plant extracts.

In one embodiment an oral composition is provided comprising an extract of at least two plant secondary metabolites, wherein said composition comprises at least about 10% (w/w) by dry weight of said extract, or at least about 9% (w/w) by dry weight of the composition, optionally said composition comprises up to about 85% (w/w) by dry weight of the extract or about 84% (w/w) by dry weight of the composition.

In one embodiment an oral composition is provided wherein said at least two plant secondary metabolites are selected from the group consisting of curcuminoids, glycosides, polysaccharides, polyphenols, amino acids, alkaloids, polysaccharides, terpenes and glycosides, preferably said composition comprises at least one SM which is not a polysaccharide.

In one embodiment an oral composition is provided wherein said plant secondary metabolite is selected from the group consisting of: rosavins, curcuminoids, saponins, beta glucan, withanolides, flavonoids, amino acids, flavonoids, ginsenosides, flavonoid glycosides, rosmarinic acid, hydroxycinnamic acid, valerenic acid, vitexin, flavanols, flavonoid glycoside, saponins, theobromine, anthocyanidins, gingerol, inulin, piperine, pectin, flavanones, flavones, boswellic acids, flavonoids, glycosides, flavanols and α-pinene and wherein said plant secondary metabolites are provided by at least two plant extracts, optionally extracted from one or more of the plants selected from the list consisting of: Rose root, Turmeric, Gotu Kola, Lion's Mane, Ashwaganda, Green Tea, Griffonia seed, Jujube, Sage, Hops flower, Oat, Allium Sativum (Garlic), Bacopa monnieri, Black pepper, Blueberry, Citrus Sinensis, Curcuma, Buckwheat Seeds, Fenugreek, cocoa, Grape, Ginger, Black Garlic, Dandelion, Orange, Panax ginseng, Barley, Grape seed, Bitter Melon, baobab, lemon skin, grapefruit, Gingko, Indian Frankincense, European Bilberry, Boswellia, Melissa officinalis, Mormodica charantia, Passiflora incarnata, Prunus cerasus, St. John's wort, Theobroma cacao and Valeriana officinalis.

In one embodiment an oral composition is provided wherein the composition further comprises a non-digestible plant fibre comprising polysaccharides, oligosaccharides, lignins or their mixtures, which non-digestible plant fibre is encapsulated in the acid resistant coating.

In one embodiment an oral composition is provided wherein said composition is encapsulated in an acid resistant coating and wherein said the encapsulation is a microencapsulation or a nanoencapsulation.

In one embodiment an oral composition is provided selected from any one of the following compositions a-e):
a) A composition comprising about 20-25% (w/w), preferably about 23% (w/w) by dry weight of the extract or about 120-200 mg, preferably about 180 mg Rose root extract and About 21-26% (w/w), preferably about 24% (w/w) by dry weight of the extract or about 130-230 mg, preferably about 190 mg Panax ginseng extract and About 5-10% (w/w), preferably 8% (w/w) by dry weight of the extract or about 180-240 mg, preferably about 210 mg St. John's wort extract and About 5-15% (w/w), preferably 10% (w/w) by dry weight of the extract or about 190-230 mg, preferably about 215 mg Melissa officinalis extract and About 5-12% (w/w), preferably 9% (w/w) by dry weight of the extract or about 420-480 mg, preferably about 450 mg Valeriana officinalis extract and About 5-12% (w/w), preferably 8% (w/w) by dry weight of the extract or about 210-260 mg, preferably about 240 mg Passiflora incarnata extract and About 15-23% (w/w), preferably 19% (w/w) by dry weight of the extract or about 120-180 mg, preferably 150 mg and About 1% (w/w) by dry weight of the extract and about 1% of an excipient, such as a filler;
b) A composition comprising about 15-25% (w/w), preferably 20% (w/w) by dry weight of the extract or about 140-180 mg, preferably about 160 mg Rose root extract and about 3-8% (w/w), preferably about 6% (w/w) by dry weight of the extract or about 40-60 mg, preferably 50 mg Grape Seed extract, and about 3-8% (w/w), preferably about 5% (w/w) by dry weight of the extract or about 30-50 mg, preferably about 40 mg Jujube extract and about 15-24% (w/w), preferably about 19% (w/w) by dry weight of the extract or about 130-170 mg, preferably 150 mg Panax ginseng extract and about 0-5% (w/w), preferably 2% (w/w) by dry weight of the extract or about 10-20 mg, preferably 15 mg Vitamin B12, and about 0-5% (w/w), preferably about 2% (w/w) by dry weight of the extract or about 35-45 mg, preferably 38 mg Vitamin D3, about 15-25% (w/w), preferably about 20% (w/w) by dry weight of the extract or about 140-170 mg, preferably 160 mg Oat extract and about 1-10% (w/w), preferably about 5% (w/w) by dry weight of the extract or about 30-60 mg, preferably 40 mg Sage extract and about 5-15% (w/w), preferably about 10% (w/w) by dry weight of the extract or about 70-100 mg, preferably 80 mg Gotu Kola extract and about 5-15% (w/w), preferably about 10% (w/w) by dry weight of the extract or about 70-100 mg, preferably 80 mg Green tea extract and about 1% of an excipient, such as a filler;
c) A composition comprising about 15-24% (w/w), preferably about 19% (w/w) by dry weight of the extract or about 140-160 mg, preferably 150 mg Fenegreek extract and about 2-8% (w/w), preferably about 8% (w/w) by dry weight of the extract or about 30-70 mg, preferably 50 mg Green Tea extract and about 0-1% (w/w), preferably about 0.5% (w/w) by dry weight of the extract or about 0-3 mg, preferably about 1.8 mg Black Peper extract and about 25-35% (w/w), preferably about 30% (w/w) by dry weight of the extract or about 230-250 mg, preferably 240 mg Lemon Skin extract and about 5-15% (w/w), preferably about 10% (w/w) by dry weight of the extract or about 70-90 mg, preferably 80 mg Grapefruit extract and about 10-20% (w/w), preferably about 15% (w/w) by dry weight of the extract or about 140-160 mg, preferably 150 mg Mormodica Charantia extract and about 10-20% (w/w), preferably about 15% (w/w) by dry weight of the extract or about 110-130 mg, preferably 120 mg Gotu Kola extract and about 1% of an excipient, such as a filler;
d) A composition comprising about 15-25% (w/w), preferably about 20% (w/w) by dry weight of the extract or about 150-170 mg, preferably 160 mg Bacopa monnieri extract and about 3-10% (w/w), preferably about 6% (w/w) by dry weight of the extract or about 40-60 mg, preferably 50 mg Boswellia serrata extract and comprising about 15-25% (w/w), preferably about 20% (w/w) by dry weight of the extract or about 150-170 mg, preferably 160 mg gingko biloba extract and about 5-15% (w/w), preferably about 10% (w/w) by dry weight of the extract or about 70-90 mg, preferably 80 mg Blueberry extract and about 3-8% (w/w), preferably about 5% (w/w) by dry weight of the extract or about 30-50 mg, preferably 40 mg Hops Flower extract and about 0-5% (w/w), preferably 2% (w/w) by dry weight of the extract or about 10-20 mg, preferably 16 mg Vitamin B6, and about 0-5% (w/w), preferably about 2% (w/w) by dry weight of the extract or about 400 μg, preferably 38 mg Vitamin B12, about 15-25% (w/w), preferably about 20% (w/w) by dry weight of the extract or about 150-170 mg, preferably 160 mg Bacopa Oat extract and about 1% of an excipient, such as a filler;
e) A composition comprising about 5-15% (w/w), preferably about 9% (w/w) by dry weight of the extract or about 60-80 mg, preferably 70 mg Blueberry extract and 5-15% (w/w), preferably about 10% (w/w) by dry weight of the extract or about 60-90 mg, preferably 80 mg Curcuma extract and 5-15% (w/w), preferably about 8% (w/w) by dry weight of the extract or about 50-80 mg, preferably 65 mg Ginger extract, and about 20-30% (w/w), preferably about 24% (w/w) by dry weight of the extract or about 170-200 mg, preferably 190 mg Rosa canina extract, and about 10-20% (w/w), preferably about 16% (w/w) by dry weight of the extract or about 120-140 mg, preferably 130 mg Malpighia glabra extract and about 5-15% (w/w), preferably about 9% (w/w) by dry weight of the extract or about 60-80 mg, preferably 70 mg Allium Sativum extract and about 5-10% (w/w), preferably about 8% (w/w) by dry weight of the extract or about 50-70 mg, preferably 60 mg Citrus Sinensis extract and about 1% of an excipient, such as a filler.

In one embodiment a composition as disclosed herein may be used in treating or alleviating dysbiosis and/or loss of microbial diversity in the gut microbiota which composition comprises an extract comprising at least two plant secondary metabolites, which are encapsulated in an acid resistant coating; wherein said extract is a mixture of several extracts.

One aspect relates to a method of increasing or decreasing the relative population of one or more bacteria genus relative population of bacteria genus (bacteria genus abundancy) in the
intestines of a vertebrate animal, the method comprising the steps of:
selecting a genus or a species of intestinal bacteria genus to be increased or decreased;
providing a composition comprising an extract containing two or more SMs, wherein the SMs constitute at least 10% (w/w) by dry weight of the extract, wherein two or more SMs are encapsulated in an acid resistant coating;
orally administering to the composition to the vertebrate animal.

One aspect of the present disclosure relates to a method of increasing the relative population of beneficial bacteria and/or decreasing the relative population of bacteria with a negative impact on health in the intestines of a vertebrate animal, the method comprising the steps of:
a) selecting a genus or a species of beneficial intestinal bacteria and/or selecting a genus or a species of intestinal bacteria with a negative impact on health;
b) providing a composition comprising an extract containing two or more SMs, wherein the SMs constitute at least 10% (w/w) by dry weight of the extract, which two or more SMs are encapsulated in an acid resistant coating;
c) orally administering the composition to the vertebrate animal.

Advantageously, such a method may use a composition of SMs tailored to increase the growth of particular beneficial bacteria and/or decrease the abundance of particular bacteria that have a negative impact on health. As the profile of microorganisms in the gut vary from individual to individual, it is preferable to administer tailored compositions of SMs that may induce growth of particular under-represented beneficial bacteria and/or decrease the abundance of particular over-represented bacteria that may have a negative impact on health. Depending on the goal to be achieved, the relative abundance of one or more beneficial bacteria in the gut may be increased by the method or the relative abundance of one or more bacteria having a negative impact on health may be decreased.

In one embodiment the desired bacteria genus to increase (positively affect) or decrease (negatively affect), may be chosen, and the selection of SMs to be used in a composition as disclosed herein be based on the tables provided herein, indicating the correlation between the SM and bacteria genus abundancies. In one embodiment the desired bacteria genus to increase or decrease may be selected after analysing the microbiota status of stool sample from a human or animal subject, determining the presence and abundance of a selection of bacteria genus. The skilled person in the art will be able to determine, which bacteria abundancies are desired to be increased or decreased based on the microbiota status.

In one embodiment, the selection of SMs combination in an extract to be used in a composition affecting bacteria abundancies are selected according to the effects correlated and disclosed herein in any one of the Tables, 2, 3, 4, 5 or 6, alone or in combination. In one embodiment the selection is based on table 2. In one embodiment the selection is based on table 3. In one embodiment the selection is based on table 4. In one embodiment the selection is based on table 5. In one embodiment the selection is based on table 6.

In one embodiment, the selection of SMs combination in an extract to be used in a composition as disclosed herein is based on the effects reviewed in table 7.

In an embodiment of the method, the step of selecting a genus or a species of beneficial bacteria and/or selecting a genus or a species of intestinal bacteria with a negative impact on health involves selecting multiple genera and/or multiple species.

The methods of the present disclosure are especially applicable for individuals who need to build muscle mass, such as athletes. For these individual, a greater abundance of beneficial bacteria in the gut may lead to a better uptake of macro and/or micronutrients, and in turn a better athletic performance. The method may also be used for the general well-being of individuals. For instance, the method may be used to maintaining a lean weight, such as a Body Mass Index below 25 kg/m². Another use may be in animal husbandry, in particular for monogastric animals, such as pigs and poultry, e.g., chickens, ducks, geese, turkey etc., but also for ruminant animals, where a greater abundance of beneficial bacteria and fewer harmful bacteria in the gut of a farm animal can lead to faster growth and/or development of the farm animal.

In an embodiment, the method is used for body weight reduction in vertebrate animals, e.g., mammals or human individuals. In an embodiment, the method is for treating colon-related dysfunction. In an embodiment, the method may be used for a general diet, or in relation to cognitive processes associated to the brain-gut-axis. In addition, the method of the present disclosure can also achieve the objects achieved by the composition of the present disclosure and/or additional objects.

In an embodiment of the method of the present disclosure, the genus of beneficial intestinal bacteria is selected from Bifidobacteria and/or Faecalibacteria and/or Bacteroides and/or Prevotella or a combination thereof and/or the genus or species of intestinal bacteria with a negative impact on health is Blautia.

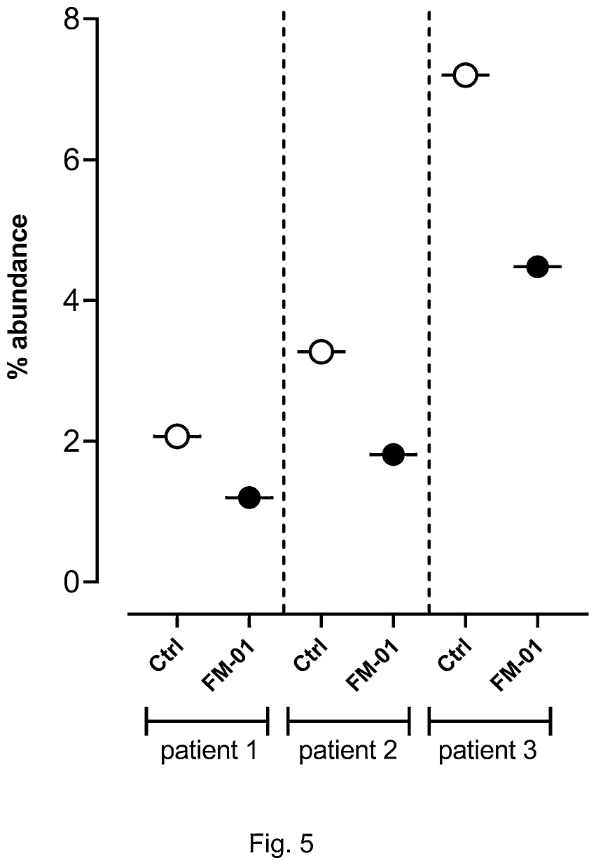

The inventor has surprisingly found that the nutraceutical composition of the invention substantially alters the composition of bacteria in the gut of vertebrate animals, especially mammals, such that beneficial bacteria represent a greater share of the total amount of bacteria in the gut and/or bacteria with a negative impact on health represent a lower share of the total amount of bacteria in the gut. Experiments have shown that such nutraceutical compositions can increase the abundance of Bifidobacteria and/or Faecalibacteria and/or Bacteroides and/or Prevotella, which are genera of bacteria that comprise important beneficial bacteria, as shown in FIGS. 1 to 4. Furthermore, experiments have shown that such nutraceutical compositions can decrease the abundance of Blautia (as shown in FIG. 5), a genus of bacteria associated with negative health impacts.

In an embodiment of the method of the present disclosure, the genus of beneficial intestinal bacteria is Bifidobacteria. In another embodiment of the method of the present disclosure, the genus of non-beneficial intestinal bacteria is Bifidobacteria. In an embodiment of the method of the present disclosure, the genus of beneficial intestinal bacteria is Faecalibacteria. In another embodiment the method of the present disclosure, the genus of non-beneficial intestinal bacteria is Faecalibacteria. In an embodiment of the method of the present disclosure, the genus of beneficial intestinal bacteria is Bacteroides. In another embodiment the method of the present disclosure, the genus of non-beneficial intestinal bacteria is Bacteroides. In an embodiment of the method of the present disclosure, the genus of beneficial intestinal bacteria is Prevotella. In another embodiment the method of the present disclosure, the genus of non-beneficial intestinal bacteria is Prevotella. In an embodiment of the method of the present disclosure, the genus of bacteria having a negative impact on health is Clostridium. In another embodiment the method of the present disclosure, the genus of beneficial intestinal bacteria is Clostridium. In an embodiment of the method of the present disclosure, the genus of bacteria having a negative impact on health is Rumonococcus. In another embodiment the method of the present disclosure, the genus of beneficial intestinal bacteria is Rumonococcus. In an embodiment of the method of the present disclosure, the genus of bacteria having a negative impact on health is Blautia. In another embodiment the method of the present disclosure, the genus of beneficial intestinal bacteria is Blautia. In an embodiment of the method of the present disclosure, the genus of bacteria having a negative impact on health is Akkermansia. In another embodiment the method of the present disclosure, the genus of beneficial intestinal bacteria is Akkermansia.

In an embodiment of the method of the present disclosure, the genus of bacteria having a negative impact on health is Coprococcus. In another embodiment the method of the present disclosure, the genus of beneficial intestinal bacteria is Coprococcus. In an embodiment of the method of the present disclosure, the genus of bacteria having a negative impact on health is Prevotella. In another embodiment the method of the present disclosure, the genus of beneficial intestinal bacteria is Prevotella. In an embodiment of the method of the present disclosure, the genus of bacteria having a negative impact on health is Rosburia. In another embodiment the method of the present disclosure, the genus of beneficial intestinal bacteria is Rosburia.

In one embodiment the method as described herein includes a step, wherein the genus of bacteria, for which and increase or decrease of abundancy is desired is selected from, Bifidobacteria, Lactobacillus, Bacteroides, Faecalibacterium, Roseburia, Ruminococcus, Clostridium, Coprococcus, Eubacterium, Escherichia, Streptococcus, Akkermansia, preferably Bifidobacteria, Faecalibacteria, Bacteroides, Prevotella, Clostridium, Coprococcus and Ruminococcus, or combinations thereof.

In one embodiment the method as described above is used to increase or decrease the bacteria abundance of one or more than one bacteria genus, wherein said method comprises the oral administration of a composition as described herein, and wherein the plant secondary metabolites are selected according to the desired effect and according to any one of the options a) and/or b) listed below:
a) an increase of a selected bacteria genus abundancy is facilitated by providing a composition as disclosed herein, and wherein, wherein at least one of the plant secondary metabolites is selected from the list consisting of: alkaloids, curcuminoids, glycosides, terpenes, polysaccharides and polyphenols, preferably selected from the plant secondary metabolites consisting of: piperine, theobromine, curcuminoids, flavonoids, saponins, pectin, anthocyanidins, flavanones, flavones, flavonoid, gingerol, withanolides, α-pinene, beta-glucan, boswellic acid and ginsenosides. Thus, all coming from the class A categorizations of alkaloids, curcuminoids, glycosides, terpenes, polysaccharides and polyphenols.

TABLE A
Compositions illustrating selected embodiments of the invention. This table indicates
the secondary metabolites (SMs) by their class A categorisation (see Definitions
and examples for further details) (the table splits across several pages). All
% (w/w) indications should be understood as the minimum content of said SMs or
Total SMs and the Extract from the Extract Source comprises other SMs in lower
concentrations, which will increase the Total SMs % (w/w) by 1-5% (w/w) depending
on the Extract Source and extraction method.(Table splits across pages)

| | | | SM class A % | Total SMs % |
|---|---|---|---|---|
| Composition | | | (w/w) in | (w/w) in |
| [ID] | Extract Source | SM class A | composition | composition |
| CM01 | rhodiola rosea | glycosides | 0.99 | 26.6 |
| CM01 | curcuma longa | curcuminoids | 18.75 | 26.6 |
| CM01 | centella asiatica | terpenes | 1.64 | 26.6 |
| CM01 | hericium erinaceus | polysaccharides | 3.95 | 26.6 |
| CM01 | riboflavin | vitamins | 1.30 | 26.6 |
| CM01 | pyridoxine | vitamins | 1.30 | 26.6 |
| CM01 | cholecalciferol | vitamins | 1.30 | 26.6 |
| CM02 | rhodiola rosea | glycosides | 0.68 | 22.3 |
| CM02 | withania somnifera | terpenes | 1.48 | 22.3 |
| CM02 | curcuma longa | curcuminoids | 12.89 | 22.3 |
| CM02 | centella asiatica | terpenes | 1.48 | 22.3 |
| CM02 | camellia sinensis | polyphenols | 6.78 | 22.3 |
| CM02 | cyanocobalamin | vitamins | 0.50 | 22.3 |
| CM02 | pyridoxine | vitamins | 0.50 | 22.3 |
| CM02 | cholecalciferol | vitamins | 0.50 | 22.3 |
| CM03 | rhodiola rosea | glycosides | 0.54 | 24.4 |
| CM03 | withania somnifera | terpenes | 0.39 | 24.4 |
| CM03 | griffonia simplicifolia | amino acids | 9.80 | 24.4 |
| CM03 | ziziphus jujuba | polyphenols | 8.23 | 24.4 |
| CM03 | avena sativa L | polysaccharides | 4.48 | 24.4 |
| CM03 | humulus lupulus | polyphenols | 8.23 | 24.4 |
| CM03 | cyanocobalamin | vitamins | 0.93 | 24.4 |
| CM03 | riboflavin | vitamins | 0.93 | 24.4 |
| CM03 | pyridoxine | vitamins | 0.93 | 24.4 |
| CM03 | avena sativa L | polysaccharides | 7.00 | 29.0 |
| CM03 | camellia sinensis | polyphenols | 8.23 | 24.4 |
| F037 | rhodiola rosea | glycosides | 0.70 | 23.9 |
| F037 | withania somnifera | terpenes | 0.47 | 23.9 |
| F037 | hypericum perforatum | glycosides | 0.70 | 23.9 |
| F037 | melissa officinalis | polyphenols | 22.70 | 23.9 |
| F038 | nu-mag rice extract | filling agent | 1.25 | 23.9 |
| F038 | valeriana officinalis | polyphenols | 22.70 | 23.9 |
| F038 | passiflora incarnata | polyphenols | 22.70 | 23.9 |
| F039 | avena sativa L | polysaccharides | 7.00 | 21.0 |
| F039 | fagopyrum esculentum | glycosides | 21.25 | 29.0 |
| F040 | avena sativa L | polysaccharides | 13.25 | 43.3 |
| F040 | trigonella foenum-graecum | glycosides | 21.25 | 29.0 |
| F041 | theobroma cacao | alkaloids | 0.80 | 29.0 |
| F041 | fagopyrum esculentum | glycosides | 28.05 | 30.7 |
| F042 | trigonella foenum-graecum | glycosides | 28.05 | 30.7 |
| F042 | theobroma cacao | alkaloids | 2.64 | 30.7 |
| F042 | trigonella foenum-graecum | glycosides | 25.00 | 29.0 |
| F042 | theobroma cacao | alkaloids | 4.00 | 29.0 |
| F043 | fagopyrum esculentum | glycosides | 17.50 | 21.5 |
| F043 | theobroma cacao | alkaloids | 4.00 | 21.5 |
| F043 | fagopyrum esculentum | glycosides | 42.50 | 42.5 |
| F044 | trigonella foenum-graecum | glycosides | 42.50 | 42.5 |
| F044 | avena sativa L | polysaccharides | 7.00 | 20.5 |
| F045 | vitis vinifera | polyphenols | 14.00 | 21.0 |
| F045 | zingiber officinale | polyphenols | 14.00 | 21.0 |
| F046 | allium sativum | polyphenols | 14.00 | 21.0 |
| F046 | vitis vinifera | polyphenols | 18.48 | 18.5 |
| F047 | zingiber officinale | polyphenols | 18.48 | 18.5 |
| F047 | allium sativum | polyphenols | 18.48 | 18.5 |
| F047 | zingiber officinale | polyphenols | 15.50 | 15.5 |
| F047 | allium sativum | polyphenols | 15.50 | 15.5 |
| F048 | vitis vinifera | polyphenols | 15.00 | 15.0 |
| F048 | zingiber officinale | polyphenols | 15.00 | 15.0 |
| F048 | vitis vinifera | polyphenols | 25.50 | 25.5 |
| F049 | allium sativum | polyphenols | 25.50 | 25.5 |
| F049 | avena sativa L | polysaccharides | 9.24 | 27.1 |
| F050 | tumeric rhizome | curcuminoids | 23.75 | 43.3 |
| F050 | taraxacum officinale | polysaccharides | 13.25 | 43.3 |
| F051 | citrus sinensis | polyphenols | 6.25 | 43.3 |
| F051 | tumeric rhizome | curcuminoids | 31.35 | 47.9 |
| F052 | taraxacum officinale | polysaccharides | 8.25 | 47.9 |
| F052 | citrus sinensis | polyphenols | 8.25 | 47.9 |
| F052 | tumeric rhizome | curcuminoids | 47.50 | 60.0 |
| F053 | taraxacum officinale | polysaccharides | 12.50 | 60.0 |
| F053 | taraxacum officinale | polysaccharides | 12.50 | 25.0 |
| F053 | citrus sinensis | polyphenols | 12.50 | 25.0 |
| F054 | tumeric rhizome | curcuminoids | 47.50 | 60.0 |
| F054 | citrus sinensis | polyphenols | 12.50 | 60.0 |
| F055 | panax ginseng | glycosides | 2.50 | 17.8 |
| F055 | vaccinium myrtillus | polyphenols | 15.25 | 17.8 |
| F056 | vitis vinifera | polyphenols | 15.25 | 17.8 |
| F056 | panax ginseng | glycosides | 3.30 | 23.4 |
| F056 | vaccinium myrtillus | polyphenols | 20.13 | 23.4 |
| F057 | vitis vinifera | polyphenols | 20.13 | 23.4 |
| F057 | panax ginseng | glycosides | 5.00 | 17.5 |
| F057 | vitis vinifera | polyphenols | 12.50 | 17.5 |
| F058 | panax ginseng | glycosides | 5.00 | 23.0 |
| F058 | vaccinium myrtillus | polyphenols | 18.00 | 23.0 |
| F059 | vitis vinifera | polyphenols | 30.00 | 42.5 |
| F059 | trigonella foenum-graecum | glycosides | 12.50 | 42.5 |
| F060 | vitis vinifera | polyphenols | 30.00 | 42.5 |
| F060 | vitis vinifera | polyphenols | 39.60 | 56.1 |
| F060 | trigonella foenum-graecum | glycosides | 16.50 | 56.1 |
| F061 | vitis vinifera | polyphenols | 39.60 | 56.1 |
| F061 | vitis vinifera | polyphenols | 47.50 | 72.5 |
| F061 | trigonella foenum-graecum | glycosides | 25.00 | 72.5 |
| F062 | trigonella foenum-graecum | glycosides | 25.00 | 37.5 |
| F062 | vitis vinifera | polyphenols | 12.50 | 37.5 |
| F062 | vitis vinifera | polyphenols | 30.00 | 42.5 |
| F063 | trigonella foenum-graecum | glycosides | 12.50 | 42.5 |
| F063 | vitis vinifera | polyphenols | 30.00 | 42.5 |
| F064 | momordica charantia | glycosides | 13.50 | 37.3 |
| F064 | trigonella foenum-graecum | glycosides | 13.50 | 37.3 |
| F065 | piper nigrum | alkaloids | 23.75 | 37.3 |
| F065 | momordica charantia | glycosides | 17.82 | 50.2 |
| F066 | trigonella foenum-graecum | glycosides | 17.82 | 50.2 |
| F066 | camellia sinensis | polyphenols | 32.34 | 50.2 |
| F067 | trigonella foenum-graecum | glycosides | 25.00 | 72.5 |
| F067 | piper nigrum | alkaloids | 47.50 | 72.5 |
| F067 | momordica charantia | glycosides | 2.00 | 49.5 |
| F068 | piper nigrum | alkaloids | 47.50 | 49.5 |
| F068 | momordica charantia | glycosides | 27.00 | 27.0 |
| F068 | trigonella foenum-graecum | glycosides | 27.00 | 27.0 |
| F069 | trigonella foenum-graecum | glycosides | 25.00 | 74.0 |
| F069 | camellia sinensis | polyphenols | 49.00 | 74.0 |
| F069 | momordica charantia | glycosides | 13.50 | 20.5 |
| F070 | avena sativa L | polysaccharides | 17.49 | 49.8 |
| F070 | trigonella foenum-graecum | glycosides | 13.50 | 20.5 |
| F070 | momordica charantia | glycosides | 17.82 | 26.1 |
| F071 | trigonella foenum-graecum | glycosides | 17.82 | 26.1 |
| F071 | taraxacum officinale | polysaccharides | 8.25 | 26.1 |
| F071 | momordica charantia | glycosides | 17.82 | 27.1 |
| F072 | avena sativa L | polysaccharides | 24.50 | 48.3 |
| F072 | trigonella foenum-graecum | glycosides | 17.82 | 27.1 |
| F072 | avena sativa L | polysaccharides | 5.60 | 22.4 |
| F073 | camellia sinensis | polyphenols | 32.34 | 49.8 |
| F073 | taraxacum officinale | polysaccharides | 17.49 | 49.8 |
| F073 | avena sativa L | polysaccharides | 7.32 | 16.9 |
| F074 | piper nigrum | alkaloids | 23.75 | 48.3 |
| F074 | citrus limon | polysaccharides | 24.50 | 48.3 |
| F075 | piper nigrum | alkaloids | 23.75 | 66.0 |
| F075 | citrus limon | polysaccharides | 17.50 | 66.0 |
| F075 | citrus paradisi | polyphenols | 24.75 | 66.0 |
| F076 | citrus limon | polysaccharides | 29.70 | 62.4 |
| F076 | citrus paradisi | polyphenols | 32.67 | 62.4 |
| F077 | adansonia | polysaccharides | 29.70 | 62.4 |
| F077 | piper nigrum | alkaloids | 47.50 | 57.5 |
| F077 | adansonia | polysaccharides | 10.00 | 57.5 |
| F078 | theobroma cacao | alkaloids | 2.64 | 58.4 |
| F078 | citrus limon | polysaccharides | 23.10 | 58.4 |
| F079 | citrus paradisi | polyphenols | 32.67 | 58.4 |
| F079 | trigonella foenum-graecum | glycosides | 25.00 | 35.0 |
| F079 | adansonia | polysaccharides | 10.00 | 35.0 |
| F080 | trigonella foenum-graecum | glycosides | 16.50 | 46.2 |
| F080 | citrus limon | polysaccharides | 29.70 | 46.2 |
| F080 | adansonia | polysaccharides | 29.70 | 46.2 |
| F080 | trigonella foenum-graecum | glycosides | 25.00 | 60.0 |
| F081 | citrus limon | polysaccharides | 35.00 | 60.0 |
| F081 | trigonella foenum-graecum | glycosides | 16.50 | 50.5 |
| F081 | theobroma cacao | alkaloids | 33.99 | 50.5 |
| F081 | piper nigrum | alkaloids | 33.99 | 50.5 |
| F081 | trigonella foenum-graecum | glycosides | 12.50 | 58.8 |
| F081 | piper nigrum | alkaloids | 23.75 | 58.8 |
| F082 | citrus limon | polysaccharides | 22.50 | 58.8 |
| F082 | adansonia | polysaccharides | 22.50 | 58.8 |
| F082 | momordica charantia | glycosides | 13.47 | 44.5 |
| F082 | trigonella foenum-graecum | glycosides | 13.47 | 44.5 |
| F082 | theobroma cacao | alkaloids | 1.24 | 44.5 |
| F082 | piper nigrum | alkaloids | 1.24 | 44.5 |
| F082 | citrus limon | polysaccharides | 17.45 | 44.5 |
| F083 | citrus paradisi | polyphenols | 12.35 | 44.5 |
| F083 | momordica charantia | glycosides | 10.87 | 47.7 |
| F083 | trigonella foenum-graecum | glycosides | 10.87 | 47.7 |
| F083 | theobroma cacao | alkaloids | 1.43 | 47.7 |
| F083 | camellia sinensis | polyphenols | 19.66 | 47.7 |
| F083 | piper nigrum | alkaloids | 1.43 | 47.7 |
| F083 | citrus limon | polysaccharides | 15.71 | 47.7 |
| F084 | citrus paradisi | polyphenols | 19.66 | 47.7 |
| F084 | momordica charantia | glycosides | 13.47 | 46.7 |
| F084 | trigonella foenum-graecum | glycosides | 13.47 | 46.7 |
| F084 | theobroma cacao | alkaloids | 1.04 | 46.7 |
| F084 | camellia sinensis | polyphenols | 14.74 | 46.7 |
| F084 | piper nigrum | alkaloids | 1.04 | 46.7 |
| F084 | citrus limon | polysaccharides | 17.46 | 46.7 |
| FM01 | citrus paradisi | polyphenols | 14.74 | 46.7 |
| FM01 | momordica charantia | glycosides | 10.13 | 51.6 |
| FM01 | trigonella foenum-graecum | glycosides | 10.13 | 51.6 |
| FM01 | theobroma cacao | alkaloids | 1.24 | 51.6 |
| FM01 | camellia sinensis | polyphenols | 18.45 | 51.6 |
| FM01 | piper nigrum | alkaloids | 1.24 | 51.6 |
| FM01 | citrus limon | polysaccharides | 21.82 | 51.6 |
| FM01 | citrus paradisi | polyphenols | 18.45 | 51.6 |
| FM01 | bacopa monnieri | glycosides | 1.34 | 20.9 |
| FM01 | ginko biloba | polyphenols | 11.20 | 20.9 |
| FM02 | boswellia serrata | terpenes | 3.62 | 20.9 |
| FM02 | vaccinium myrtillus | polyphenols | 11.20 | 20.9 |
| FM02 | vitis vinifera | polyphenols | 11.20 | 20.9 |
| FM02 | ziziphus jujuba | polyphenols | 11.20 | 20.9 |
| FM02 | humulus lupulus | polyphenols | 11.20 | 20.9 |
| FM02 | cyanocobalamin | vitamins | 0.60 | 20.9 |
| FM02 | pyridoxine | vitamins | 0.60 | 20.9 |
| FM02 | hordeum vulgare | polysaccharides | 4.18 | 20.9 |
| FM02 | bacopa monnieri | glycosides | 1.38 | 21.3 |
| FM02 | ginko biloba | polyphenols | 12.13 | 21.3 |
| FM02 | boswellia serrata | terpenes | 3.99 | 21.3 |
| FM3 | vaccinium myrtillus | polyphenols | 12.13 | 21.3 |
| FM3 | centella asiatica | terpenes | 3.99 | 21.3 |
| FM3 | vitis vinifera | polyphenols | 12.13 | 21.3 |
| FM3 | panax ginseng | glycosides | 1.38 | 21.3 |
| FM3 | cyanocobalamin | vitamins | 0.55 | 21.3 |
| FM3 | riboflavin | vitamins | 0.55 | 21.3 |
| FM3 | cholecalciferol | vitamins | 0.55 | 21.3 |
| FM3 | hordeum vulgare | polysaccharides | 3.29 | 21.3 |
| FM3 | bacopa monnieri | glycosides | 2.85 | 22.4 |
| FM30 | humulus lupulus | polyphenols | 6.25 | 22.4 |
| FM30 | cyanocobalamin | vitamins | 3.43 | 22.4 |
| FM30 | pyridoxine | vitamins | 3.43 | 22.4 |
| FM30 | avena sativa L | polysaccharides | 9.74 | 23.2 |
| FM30 | panax ginseng | glycosides | 2.85 | 22.4 |
| FM30 | vaccinium myrtillus | polyphenols | 6.25 | 22.4 |
| FM30 | boswellia serrata | terpenes | 4.23 | 22.4 |
| FM30 | ginko biloba | polyphenols | 6.25 | 22.4 |
| FM31 | bacopa monnieri | glycosides | 3.56 | 20.9 |
| FM31 | humulus lupulus | polyphenols | 7.81 | 20.9 |
| FM31 | cyanocobalamin | vitamins | 4.29 | 20.9 |
| FM31 | pyridoxine | vitamins | 4.29 | 20.9 |
| FM31 | panax ginseng | glycosides | 3.56 | 20.9 |
| FM32 | vaccinium myrtillus | polyphenols | 7.81 | 20.9 |
| FM32 | boswellia serrata | terpenes | 5.28 | 20.9 |
| FM32 | ginko biloba | polyphenols | 7.81 | 20.9 |
| FM32 | bacopa monnieri | glycosides | 3.73 | 16.9 |
| MEM2 | humulus lupulus | polyphenols | 0.33 | 16.9 |
| MEM2 | avena sativa L | polysaccharides | 5.60 | 20.5 |
| MEM2 | panax ginseng | glycosides | 3.73 | 16.9 |
| MEM2 | boswellia serrata | terpenes | 5.52 | 16.9 |
| MEM3 | avena sativa L | polysaccharides | 14.00 | 46.8 |
| MEM3 | panax ginseng | glycosides | 3.04 | 23.2 |
| MEM3 | vaccinium myrtillus | polyphenols | 10.43 | 23.2 |
| MEM3 | ginko biloba | polyphenols | 10.43 | 23.2 |
| MEM4 | nu-mag rice extract | filling agent | 1.00 | 20.5 |
| MEM4 | bacopa monnieri | glycosides | 2.40 | 20.5 |
| MEM4 | panax ginseng | glycosides | 2.40 | 20.5 |
| MEM4 | ginko biloba | polyphenols | 7.55 | 20.5 |
| MEM5 | humulus lupulus | polyphenols | 7.55 | 20.5 |
| MEM5 | vaccinium myrtillus | polyphenols | 7.55 | 20.5 |
| MEM5 | avena sativa L | polysaccharides | 7.00 | 67.8 |
| MEM5 | boswellia serrata | terpenes | 3.90 | 20.5 |
| MEM6 | cyanocobalamin | vitamins | 2.01 | 20.5 |
| MEM6 | pyridoxine | vitamins | 2.01 | 20.5 |
| MEM6 | vaccinium myrtillus | polyphenols | 5.45 | 18.7 |
| MEM6 | tumeric rhizome | curcuminoids | 2.60 | 18.7 |
| MEM6 | zingiber officinale | polyphenols | 5.45 | 18.7 |
| MEM7 | malpighia glabra | vitamins | 15.04 | 18.7 |
| MEM7 | nu-mag rice extract | filling agent | 1.00 | 18.7 |
| MEM7 | allium sativum | polyphenols | 2.34 | 18.7 |
| MEM7 | citrus sinensis | polyphenols | 5.45 | 18.7 |
| MEM7 | rosa canina | vitamins | 15.04 | 18.7 |
| MM11 | chicorium intybus | polysaccharides | 2.25 | 18.7 |
| MM11 | trigonella foenum-graecum | glycosides | 12.37 | 54.1 |
| MM11 | camellia sinensis | polyphenols | 16.02 | 54.1 |
| MM11 | piper nigrum | alkaloids | 4.71 | 54.1 |
| MM11 | citrus limon | polysaccharides | 20.99 | 54.1 |
| MSRM1 | citrus paradisi | polyphenols | 16.02 | 54.1 |
| MSRM1 | mormodica charantia | glycosides | 12.37 | 54.1 |
| MSRM1 | nu-mag rice extract | filling agent | 1.00 | 54.1 |
| MSRM1 | theobroma cacao | alkaloids | 4.71 | 54.1 |
| NPM1 | rhodiola rosea | glycosides | 1.75 | 2.2 |
| NPM1 | bacopa monnieri | glycosides | 1.75 | 2.2 |
| NPM1 | withania somnifera | terpenes | 0.45 | 2.2 |
| NPM1 | centella asiatica | terpenes | 0.45 | 2.2 |
| NPM2 | bacopa monnieri | glycosides | 1.00 | 23.6 |
| NPM2 | withania somnifera | terpenes | 16.58 | 23.6 |
| NPM2 | ginko biloba | polyphenols | 6.00 | 23.6 |
| NPM2 | boswellia serrata | terpenes | 16.58 | 23.6 |
| NPM3 | rhodiola rosea | glycosides | 0.43 | 21.9 |
| NPM3 | hericium erinaceus | polysaccharides | 11.43 | 21.9 |
| NPM3 | camellia sinensis | polyphenols | 9.14 | 21.9 |
| NPM3 | curcuma longa | curcuminoids | 0.86 | 21.9 |
| NPM4 | withania somnifera | terpenes | 0.37 | 16.1 |
| NPM4 | ginko biloba | polyphenols | 3.43 | 16.1 |
| NPM4 | hericium erinaceus | polysaccharides | 11.43 | 16.1 |
| NPM4 | curcuma longa | curcuminoids | 0.86 | 16.1 |
| NPM5 | rhodiola rosea | glycosides | 0.38 | 15.5 |
| NPM5 | withania somnifera | terpenes | 0.33 | 15.5 |
| NPM5 | hericium erinaceus | polysaccharides | 10.00 | 15.5 |
| NPM5 | camellia sinensis | polyphenols | 4.00 | 15.5 |
| NPM6 | curcuma longa | curcuminoids | 0.75 | 15.5 |
| NPM6 | rhodiola rosea | glycosides | 0.67 | 8.8 |
| NPM6 | withania somnifera | terpenes | 0.34 | 8.8 |
| NPM6 | centella asiatica | terpenes | 0.34 | 8.8 |
| NPM7 | camellia sinensis | polyphenols | 7.11 | 8.8 |
| NPM7 | curcuma longa | curcuminoids | 0.67 | 8.8 |
| NPM7 | ginko biloba | polyphenols | 6.26 | 15.4 |
| NPM7 | centella asiatica | terpenes | 1.74 | 15.4 |
| PG01 | hericium erinaceus | polysaccharides | 2.61 | 15.4 |
| PG01 | panax ginseng | glycosides | 0.52 | 15.4 |
| PG01 | riboflavin | vitamins | 4.26 | 15.4 |
| PG01 | rhodiola rosea | glycosides | 0.75 | 41.3 |
| PG01 | camellia sinensis | polyphenols | 8.00 | 41.3 |
| PG01 | boswellia serrata | terpenes | 32.50 | 41.3 |
| PG01 | boswellia serrata | terpenes | 32.50 | 41.3 |
| PG01 | bacopa monnieri | glycosides | 1.00 | 21.1 |
| PG02 | centella asiatica | terpenes | 0.13 | 21.1 |
| PG02 | hericium erinaceus | polysaccharides | 10.00 | 21.1 |
| PG02 | vitis vinifera | polyphenols | 10.00 | 21.1 |
| PG02 | bacopa monnieri | glycosides | 1.00 | 25.0 |
| PG02 | ginko biloba | polyphenols | 24.00 | 25.0 |
| PG02 | camellia sinensis | polyphenols | 24.00 | 25.0 |
| PG02 | vitis vinifera | polyphenols | 24.00 | 25.0 |
| PG02 | rhodiola rosea | glycosides | 0.75 | 9.2 |
| PG02 | withania somnifera | terpenes | 0.45 | 9.2 |
| PG02 | centella asiatica | terpenes | 0.45 | 9.2 |
| PG02 | camellia sinensis | polyphenols | 8.00 | 9.2 |
| PG03 | bacopa monnieri | glycosides | 0.57 | 30.7 |
| PG03 | centella asiatica | terpenes | 18.71 | 30.7 |
| PG03 | boswellia serrata | terpenes | 18.71 | 30.7 |
| PG03 | vitis vinifera | polyphenols | 11.43 | 30.7 |
| PG03 | centella asiatica | terpenes | 21.83 | 29.0 |
| PG03 | hericium erinaceus | polysaccharides | 6.67 | 29.0 |
| PG03 | curcuma longa | curcuminoids | 0.50 | 29.0 |
| PG03 | boswellia serrata | terpenes | 21.83 | 29.0 |
| PG04 | bacopa monnieri | glycosides | 1.00 | 17.1 |
| PG04 | centella asiatica | terpenes | 0.13 | 17.1 |
| PG04 | ginko biloba | polyphenols | 16.00 | 17.1 |
| PG04 | vitis vinifera | polyphenols | 16.00 | 17.1 |
| PG04 | ginko biloba | polyphenols | 16.00 | 42.3 |
| PG04 | hericium erinaceus | polysaccharides | 10.00 | 42.3 |
| PG04 | boswellia serrata | terpenes | 16.25 | 42.3 |
| PG04 | vitis vinifera | polyphenols | 16.00 | 42.3 |
| PG04 | vitis vinifera | polyphenols | 32.75 | 46.8 |
| PG04 | avena sativa L | polysaccharides | 14.00 | 62.3 |
| PG05 | vaccinium myrtillus | polyphenols | 32.75 | 46.8 |
| PG05 | curcuma longa | curcuminoids | 23.75 | 81.0 |
| PG05 | vitis vinifera | polyphenols | 57.25 | 81.0 |
| PG05 | vaccinium myrtillus | polyphenols | 57.25 | 81.0 |
| PG05 | camellia sinensis | polyphenols | 57.25 | 81.0 |
| PG05 | curcuma longa | curcuminoids | 17.81 | 67.8 |
| PG05 | vitis vinifera | polyphenols | 42.94 | 67.8 |
| PG05 | avena sativa L | polysaccharides | 5.60 | 26.2 |
| PG05 | vaccinium myrtillus | polyphenols | 42.94 | 67.8 |
| PPM10 | camellia sinensis | polyphenols | 42.94 | 67.8 |
| PPM10 | curcuma longa | curcuminoids | 23.75 | 62.3 |
| PPM10 | avena sativa L | polysaccharides | 8.43 | 10.4 |
| PPM12 | camellia sinensis | polyphenols | 24.50 | 62.3 |
| PPM12 | rhodiola rosea | glycosides | 0.60 | 26.2 |
| PPM12 | ziziphus jujuba | polyphenols | 15.91 | 26.2 |
| PPM12 | humulus lupulus | polyphenols | 15.91 | 26.2 |
| PPM120 | cyanocobalamin | vitamins | 2.55 | 26.2 |
| PPM120 | riboflavin | vitamins | 2.55 | 26.2 |
| PPM120 | avena sativa L | polysaccharides | 10.98 | 43.8 |
| PPM120 | withania somnifera | terpenes | 1.57 | 26.2 |
| PPM120 | vitis vinifera | polyphenols | 15.91 | 26.2 |
| PPM20 | avena sativa L | polysaccharides | 5.60 | 27.7 |
| PPM20 | centella asiatica | terpenes | 1.57 | 26.2 |
| PPM20 | camellia sinensis | polyphenols | 15.91 | 26.2 |
| RM1 | rhodiola rosea | glycosides | 0.75 | 25.8 |
| RM1 | ziziphus jujuba | polyphenols | 19.89 | 25.8 |
| RM1 | humulus lupulus | polyphenols | 19.89 | 25.8 |
| RM1 | cyanocobalamin | vitamins | 3.19 | 25.8 |
| RM1 | riboflavin | vitamins | 3.19 | 25.8 |
| RM1 | withania somnifera | terpenes | 1.96 | 25.8 |
| RM1 | vitis vinifera | polyphenols | 19.89 | 25.8 |
| RM1 | avena sativa L | polysaccharides | 14.00 | 34.0 |
| RM1 | centella asiatica | terpenes | 1.96 | 25.8 |
| RM1 | camellia sinensis | polyphenols | 19.89 | 25.8 |
| RM1 | rhodiola rosea | glycosides | 0.90 | 10.4 |
| RM10 | ziziphus jujuba | polyphenols | 0.24 | 10.4 |
| RM10 | avena sativa L | polysaccharides | 7.00 | 21.8 |
| RM10 | withania somnifera | terpenes | 0.86 | 10.4 |
| RM10 | avena sativa L | polysaccharides | 14.00 | 16.3 |
| RM10 | humulus lupulus | polyphenols | 30.88 | 43.8 |
| RM10 | prunus cerasus | polyphenols | 22.70 | 23.9 |
| RM10 | vitis vinifera | polyphenols | 30.88 | 43.8 |
| RM10 | centella asiatica | terpenes | 1.96 | 43.8 |
| RM10 | camellia sinensis | polyphenols | 30.88 | 43.8 |
| RM10 | rhodiola rosea | glycosides | 0.60 | 27.7 |
| RM11 | vitis vinifera | polyphenols | 15.65 | 27.7 |
| RM11 | ziziphus jujuba | polyphenols | 15.65 | 27.7 |
| RM11 | withania somnifera | terpenes | 1.54 | 27.7 |
| RM11 | riboflavin | vitamins | 3.96 | 27.7 |
| RM11 | cholecalciferol | vitamins | 3.96 | 27.7 |
| RM12 | salvia officinalis L | polyphenols | 8.23 | 24.4 |
| RM12 | salvia officinalis L | terpenes | 1.57 | 26.2 |
| RM12 | centella asiatica | terpenes | 1.54 | 27.7 |
| RM12 | camellia sinensis | polyphenols | 15.65 | 27.7 |
| RM12 | nu-mag rice extract | filling agent | 1.00 | 27.7 |
| TSM10 | boswellia serrata | terpenes | 17.50 | 34.0 |
| TSM10 | centella asiatica | terpenes | 17.50 | 34.0 |
| TSM10 | salvia officinalis L | terpenes | 1.96 | 25.8 |
| TSM10 | panax ginseng | glycosides | 2.50 | 34.0 |
| TSM12 | bacopa monnieri | glycosides | 3.50 | 20.7 |
| TSM12 | boswellia serrata | terpenes | 17.19 | 20.7 |
| TSM12 | centella asiatica | terpenes | 17.19 | 20.7 |
| TSM12 | panax ginseng | glycosides | 3.50 | 20.7 |
| TSM120 | bacopa monnieri | glycosides | 2.63 | 21.8 |
| TSM120 | boswellia serrata | terpenes | 12.19 | 21.8 |
| TSM120 | salvia officinalis L | terpenes | 0.86 | 10.4 |
| TSM120 | panax ginseng | glycosides | 2.63 | 21.8 |
| TSM20 | bacopa monnieri | glycosides | 1.00 | 16.3 |
| TSM20 | centella asiatica | terpenes | 1.25 | 16.3 |
| TSM20 | salvia officinalis L | terpenes | 1.54 | 16.3 |

End of Table A—Compositions illustrating selected embodiments of the invention. This table indicates the secondary metabolites by their class A categorisation (see Definitions and examples for further details) (the table splits across several pages). All % (w/w) indications should be understood as the minimum content of said SMs or Total S Ms.

Table A indicates the minimum content of the formulation of specific SMs listed in the table, although the minimum total SMs % (w/w) is shown as between 8.8 and 9.2 for NPM3 and MEM7 respectively, the total content of all SMs will result in about 10% (w/w) or more of the total extract, comprising several extracts. Since each extract used comprises additional SMs in lower concentrations than the ones specifically identified herein, the total of all SMs concentration in the single extract, as well as the extract comprising several extracts will thus be increased by the additional SMs present in said extract. Thus, the Total S Ms % (w/w) present in an extract or a composition, i.e. an extract comprising several extracts, may be increased by 1-5% (w/w) relative to the indicated value and depending on the Extract Source and extraction method. The additional SMs up to said about 10% (w/w) are provided by the less predominant SMs also present the plant extracts used, which are not specifically outlined in the table above.

In one embodiment a composition of the present invention can be selected from one or more composition of Table A. In one embodiment a composition may comprise extracts and SMs as indicated in Table A, independently selected from the group of compositions consisting of: CM01, CM02, CM03, PG01, F037, F038, F039, F040, F041, F042, F043, F044, F045, F046, F047, F048, F049, F050, F051, F052, F053, F054, F055, F056, F057, F058, F059, F060, F061, F062, F063, F064, F065, F066, F067, F068, F069, F070, F071, F072, F073, F074, F075, F076, F077, F078, F079, F080, F081, F082, F083, F084, FM01, FM02, FM3, FM30, FM31, FM32, PG04, PG05, PG03, MEM2, MEM3, MEM4, MEM5, MEM6, MEM7, MM11, MSRM1, NPM1, NPM2, NPM3, NPM4, NPM5, NPM6, NPM7, PPM10, PPM12, PPM120, PPM20, RM1, RM10, RM11, RM12, PG02, TSM10, TSM12, TSM120 and TSM20. In one embodiment an extract comprises at least two SMs independently selected from the group consisting of: polyphenols, glycosides, polysaccharides, terpenes and vitamins.

In one embodiment an extract comprises at least two SMs selected from polyphenols and glycosides, respectively. In one embodiment an extract comprises at least two SMs selected from polyphenols and polysaccharides, respectively. In one embodiment an extract comprises at least two SMs selected from polyphenols and terpenes, respectively. In one embodiment an extract comprises at least two SMs selected from polyphenols and alkaloids, respectively. In one embodiment an extract comprises at least two SMs selected from polyphenols and curcuminoids, respectively. In one embodiment an extract comprises at least two SMs selected from glycosides and vitamins, respectively. In one embodiment an extract comprises at least two SMs are selected glycosides and terpenes, respectively. In one embodiment an extract comprises at least two SMs are selected glycosides and alkaloids, respectively. In one embodiment an extract comprises at least two SMs are selected terpenes and vitamins, respectively. In one embodiment an extract comprises at least two SMs are selected curcuminoids and polysaccharides, respectively. In one embodiment an extract comprises at least two SMs are selected curcuminoids and vitamins, respectively. In one embodiment an extract comprises at least two SMs are selected polysaccharides and alkaloids, respectively. In one embodiment an extract comprises at least two SMs are selected polysaccharides and vitamins, respectively.

In one embodiment said extract comprises at least two plant secondary metabolites independently selected from one or more of the combinations a)-u): a) polyphenols and one or more other plant secondary metabolite; b) glycosides and one or more other plant secondary metabolite; d) polyphenols and glycosides; e) polyphenols and polysaccharides; f) polyphenols and vitamins; g) polyphenols and terpenes; h) polyphenols and alkaloids; i) compromising polyphenols and curcuminoids; j) glycosides and vitamins; k) glycosides and terpenes; l) glycosides and alkaloids; m) terpenes and vitamins; n) curcuminoids and polysaccharides; o) curcuminoids and vitamins; p) polysaccharides and alkaloids; q) polysaccharides and alkaloids; r) polyphenols, glycosides, terpenes and vitamins; s) polyphenols, glycosides, polysaccharides, terpenes and vitamins; t) polyphenols, glycosides, polysaccharides and alkaloids; u) polyphenols, curcuminoids, polysaccharides and vitamins.

In one embodiment an extract comprises at least two SMs, wherein said SMs are independently selected from at least two SMs of the group of SMs consisting of: polyphenols, glycosides, terpenes and vitamins. In one embodiment an extract comprises at least two SMs, wherein said SMs are independently selected from at least two SMs of the group of SMs consisting of: polyphenols, glycosides, polysaccharides, terpenes and vitamins. In one embodiment an extract comprises at least two SMs, wherein said SMs are independently selected from at least two SMs of the group of SMs consisting of: polyphenols, glycosides, polysaccharides and alkaloids. In one embodiment an extract comprises at least two SMs, wherein said SMs are independently selected from at least two SMs of the group of SMs consisting of: polyphenols, curcuminoids, polysaccharides and vitamins.

In one embodiment an extract comprises at least two SMs and comprises polyphenols, glycosides, polysaccharides, terpenes and vitamins and wherein said extract comprises at least about 20-30% (w/w), preferably about 22-28% (w/w) SMs in total, about 8-16% (w/w) polyphenols, about 0.5-2.5% (w/w) glycosides, about 6% (w/w) polysaccharides, about 2-4% (w/w) vitamins and about 4% (w/w) terpenes by dry weight of the extract.

In one embodiment an extract comprises at least two SMs and comprises polyphenols, glycosides, polysaccharides, terpenes and vitamins and wherein said extract comprises at least about 20-23% (w/w), preferably about 22% (w/w) SMs in total, about 8% (w/w) polyphenols, about 2.5% (w/w) glycosides, about 6% (w/w) polysaccharides, about 2% (w/w) vitamins and about 4% (w/w) terpenes by dry weight of the extract.

In one embodiment an extract comprises at least two SMs and comprises polyphenols, glycosides, polysaccharides, terpenes and vitamins and wherein said extract comprises at least about 27-30% (w/w), preferably about 28% SMs in total, about 0.5-1% (w/w) glycosides, preferably about 0.6% (w/w), about 16% (w/w) polyphenols, about 6% (w/w) polysaccharides, about 1.5% (w/w) terpenes about 4% (w/w) vitamins by dry weight of the extract.

In one embodiment the total weight of the extract is between 300 and 600 mg. In one embodiment the total weight of the extract is between 400 and 500 mg. In one embodiment the total weight of the extract is between 500 and 1000 mg. In one embodiment the total weight of the extract is between 900 and 1000 mg.

In one embodiment an extract comprises at least two SMs independently selected from the group consisting of: polyphenols, glycosides, terpenes and vitamins.

In one embodiment an extract comprises at least two SMs and comprises polyphenols, glycosides, polysaccharides, terpenes and vitamins and wherein said extract comprises at least 26-30% (w/w) preferably about 28% (w/w) SMs, about 8% (w/w) polyphenols, about 3% (w/w) curcuminoids, about 2% (w/w) polysaccharides and about 15% (w/w) vitamins.

In one embodiment an extract comprises at least two SMs independently selected from the group consisting of: polyphenols, curcuminoids, polysaccharides and vitamins.

In one embodiment an extract comprises at least two SMs and comprises polyphenols, glycosides, polysaccharides, terpenes and vitamins and wherein said extract comprises at least 26-30% (w/w) preferably about 28% (w/w) SMs, about 8% (w/w) polyphenols, about 3% (w/w) curcuminoids, about 2% (w/w) polysaccharides and about 15% (w/w) vitamins.

In one embodiment an extract comprises at least two SMs independently selected from the group consisting of: polyphenols, glycosides, polysaccharides and alkaloids.

In one embodiment an extract comprises at least two SMs and comprises polyphenols, about 52-57% (w/w), preferably about 54% (w/w) SMs, about 16% (w/w) polyphenols, about 21% (w/w) polysaccharides and about 5% (w/w) alkaloids and 12% (w/w) glycosides.

In one embodiment an extract comprises at least two SMs independently selected from the group consisting of: polyphenols, glycosides, polysaccharides and alkaloids.

In one embodiment an extract comprises at least two SMs independently selected from the group consisting of: polyphenols, curcuminoids, polysaccharides and vitamins.

In one embodiment a composition according to the present invention is selected from the compositions indicated as PG01 as disclosed in Example 5. In one embodiment a composition according to the present invention is selected from the compositions indicated as PG02 as disclosed in Example 5. In one embodiment a composition according to the present invention is selected from the compositions indicated as PG03 as disclosed in Example 5. In one embodiment a composition according to the present invention is selected from the compositions indicated as PG04 as disclosed in Example 5. In one embodiment a composition according to the present invention is selected from the compositions indicated as PG05 as disclosed in Example 5.

In one embodiment a composition for oral administration, the composition comprising an extract containing two or more SMs, wherein the SMs constitute at least 10% (w/w) by dry weight of the extract, which two or more SMs are encapsulated in an acid resistant coating.

In one embodiment a composition comprises two or more SMs which are from different plants. In one embodiment a composition further comprises a non-digestible plant fibre comprising polysaccharides, oligosaccharides, lignins or their mixtures, which non-digestible plant fibre is encapsulated in the acid resistant coating. In one embodiment a composition comprises two or more SMs, which are selected from a phenol, a polyphenol, a flavonoid, an anthocyanin, a terpenoid, a terpene, a saponin, an alkaloid, a lactone, a glycoside, or their mixtures. In one embodiment a composition comprises SMs comprising a phenol, a polyphenol, a flavonoid, an anthocyanin, or their mixtures and a terpenoid or a terpene or their mixtures. In one embodiment a composition comprises SMs selected from the group consisting of phenols, polyphenols, flavonoids and anthocyanins, or a mixture thereof.

In one embodiment a composition further comprises terpenoids or terpenes or their mixtures. In one embodiment a composition is encapsulation is a microencapsulation or a nanoencapsulation.

In one embodiment a composition comprises two or more SMs which are obtainable from a plant selected from the list consisting of: Bacopa monnieri, Gingko biloba, Boswellia serrata, Panax ginseng, Vitis vinifera, Vaccinium myrtillus, Ziziphus jujube, Centella asiatica, Hericium erinaceus, Rhodiola rosea, Withania somnifera, Camellia sinensis, and Curcuma longa.

In one embodiment a composition comprises two or more SMs, wherein the SMs constitute at least 10% (w/w) by dry weight of the extract, which SMs are encapsulated in an acid resistant coating, in the manufacture of a prebiotic for a vertebrate animal.

In one embodiment the invention provides a method of increasing the relative population of beneficial bacteria and/or decreasing the relative population of bacteria with a negative impact on health in the intestines of a vertebrate animal, the method comprising the steps of:
selecting a genus or a species of beneficial intestinal bacteria and/or selecting a genus or a species of intestinal bacteria with a negative impact on health;
providing a composition comprising an extract containing two or more SMs, wherein the SMs constitute at least 10% (w/w) by dry weight of the extract, which two or more SMs are encapsulated in an acid resistant coating;
orally administering the composition to the vertebrate animal.

In one embodiment said method according to this invention, is applied for the genus of beneficial intestinal bacteria is selected from Bifidobacteria, Faecalibacteria, Bacteroides, Prevotella or combinations thereof and/or the genus or species of intestinal bacteria with a negative impact on health is Blautia.

In one embodiment a composition according to this invention may comprise ingredients according to MEM-7 consists of extracts of:

| | | | | |
|---|---|---|---|---|
| rhodiola | withania | centella | camellia | curcuma |
| rosea | somnifera | asiatica | sinensis | longa |

| | |
|---|---|
| Triterpenoids | >1% |
| Polyphenols | >7.5% |

Composition MEM-7 has at least the following SMs content (% is w/w by dry weight): Total weight of composition: 999 mg

Composition NPM-2 consists of extracts of:

| | | | |
|---|---|---|---|
| bacopa monnieri | gingko biloba | vitis vinifera | camellia sinensis |

Composition NPM-2 has at least the following SMs content (% is w/w by dry weight):

| | |
|---|---|
| Triterpenoids | >0.5% |
| Polyphenols | >18% |
| Polyphenols (Flavones) | >6% |

Total weight of composition: 1000 mg

Composition MM-11 consists of extracts of:

| | | | |
|---|---|---|---|
| gingko biloba | centella asiatica | panax ginseng | hericium erinaceus |

Composition MM-11 has at least the following SMs content (% is w/w by dry weight):

| | |
|---|---|
| Triterpenoids | >30% |
| Polyphenols | >7% |
| Polyphenols (Flavones) | >6% |

Total weight of composition: 957 mg

Composition FM-01 consists of extracts of:

| | | | | | |
|---|---|---|---|---|---|
| bacopa | gingko | boswellia | vaccinium | vitis | hordeum |
| monnieri | biloba | serrata | myrtillus | vinifera | vulgare |

Composition FM-01 has at least the following SMs content (% is w/w by dry weight):

| | |
|---|---|
| Triterpenoids | >5% |
| Polyphenols | >8% |
| Beta glucan | >1.5% |
| Polyphenols (Flavones) | >5% |

Total weight of composition: 897 mg Composition NPM-1 consists of extracts of:

| | | | |
|---|---|---|---|
| bacopa monnieri | centella asiatica | hericium erinaceus | vitis vinifera |

Composition NPM-1 has at least the following SMs content (% is w/w by dry weight).

| | |
|---|---|
| Triterpenoids | >2.5% |
| Polyphenols | >10% |

Total weight of composition: 1000 mg

Composition MEM-3 consists of extracts of:

| | | | |
|---|---|---|---|
| withania | bacopa | gingko | boswellia |
| somnifera | monnieri | biloba | serrata |

Composition MEM-3 has at least the following SMs content (% is w/w by dry weight).

| | |
|---|---|
| Triterpenoids | >16% |
| Polyphenols (Flavones) | >6% |

Total weight of composition: 1000 mg

### DEFINITIONS & FURTHER EMBODIMENTS

The composition of the invention may be referred to as a “nutraceutical composition” or “prebiotic composition”, and the two terms may be used interchangeably. The prebiotic composition may be used in combination with probiotic compositions or comprise bacteria, known as probiotics. In some embodiments the compositions and/or extracts described herein may be combined with colony forming units (CFU)) providing one or more bacteria strains, also known as probiotics. In some embodiments the compositions or extracts as disclosed herein may be combined with between 10.000.000 CFU to about 80.000.000 CFU probiotics. In some embodiments the compositions or extracts as disclosed herein may be combined with between 20.000.000 CFU to about 40.000.000 CFU, preferably 30.000.000 CFU.

“Probiotics” as used herein may contain a variety of microorganisms. The most common are bacteria that belong to groups called Lactobacillus and Bifidobacterium. Other bacteria may also be used as probiotics, such as, but not limited to Saccharomyces boulardii. In one embodiment said probiotics may be selected from one, more or all of the following: Lactobacillus acidophilus, Lactobacillus plantarum, Lactobacillus casei, Lactobacillus rhamnosus, Bifidobacterium longum, Bifidobacterium lactis, and Bifidobacterium.

The skilled person knows how to quantify the SM content of the extract, for example by the relative quantification of secondary metabolites by means of high-pressure liquid chromatography (HPLC) in relation to the total amount of the extract, such as plant extract. In some instances, the HPLC may be combined with Mass Spectroscopy to identify said plant secondary plant extract in said extract. The skilled person in the art uses HPLC for Phytochemical Screening of different plants and extracts and will apply similar methods to identify the content of secondary metabolites present in the compositions as described and claimed herein.

In some embodiments plant extracts may be derived from plat, root, seed, leaf or any other part of the plants and vitamins. The following lists examples of suitable extract sources for a selection of plants: adansonia (fruit extract), Allium sativum (bulb extract), Avena sativa I (bran extract), Bacopa monnieri (whole plant extract/raw powder), Boswellia serrata (plant or resin extract), Camellia sinensis (leaf extract), Centella asiatica (whole plant extract/raw powder), Chicorium intybus (plant extract), cholecalciferol (raw powder), Citrus limon (skin extract), Citrus paradisi (fruit extract), Citrus sinensis (skin extract), Curcuma longa (raw powder/root extract), cyanocobalamin (raw powder), Fagopyrum esculentum (seed extract), ginko biloba (leaf extract), Griffonia simplicifolia (seed extract), Hericium erinaceus (raw powder), Hericium erinaceus (fungus extract), Hordeum vulgare (bran extract), Humulus lupulus (flower extract), huperzia serrata (whole plant extract), Hypericum perforatum (plant/arial part), Malpighia glabra (berry extract), Malpighia glabra (fruit extract), Melissa officinalis (leaf extract), Momordica charantia (raw powder), Mormodica charantia (fruit extract/raw powder), myristica fragrans (seed extract), Panax ginseng (root extract), panax ginseng (root extract), Passiflora incarnata (flower extract), Piper nigrum (fruit extract), Prunus cerasus (fruit extract), pyridoxine (raw powder), Rhodiola crenulata (root extract), Rhodiola rosea (root extract), Rhodiola rosea (root extract), riboflavin (raw powder), Rosa canina (fruit extract), Rosa canina (fruit extract), Salvia officinalis I (leaf extract), Sambucus nigra (fruit extract), Taraxacum officinale (root extract), Theobroma cacao (bean extract), Trigonella foenum-graecum (seed extract), Tumeric rhizome (root extract), Vaccinium myrtillus (fruit extract), Valeriana officinalis (root extract), Vitis vinifera (seed extract/skin extract), Withania somnifera (root extract), Withania somnifera (raw powder), Zingiber officinale (root extract), Ziziphus jujuba (fruit extract).

The term “plant secondary metabolite” as used herein in singular or plural may be abbreviated “SM” or “SMs” and refers to compounds that are not directly involved in the plant metabolism, e.g., the normal growth, development, or reproduction of the plant. Plant secondary metabolites are classified by their chemical structure and can be divided into four major classes: terpenes, phenolics, glycosides and alkaloids. In addition, major classes, such as amino acids, curcuminoids, polyphenols, polysaccharides and vitamins may also be understood to be plant secondary metabolites.

Further distinction can be made within these general classes mentioned above and without excluding any plant secondary metabolites not mentioned in the below list, the list shown below indicates the classification order applied in this application. Herein, class A describes a higher genus than the class B classification. All % (w/w) indications should be understood as the minimum content of said specific Class A or Class B SMs.

Since the Extract used may comprise other SMs in lower concentrations than the ones specifically identified, the SMs concentration in the single extract, as well as the extract comprising several extracts will thus be increased by the additional SMs present in said extract. Thus, the Total S Ms % (w/w) present in an extract or a composition, i.e., an extract comprising several extracts, may be increased by 1-5% (w/w) relative to the indicated value and depending on the Extract Source and extraction method. No single SMs in said composition may be present in more than 50% (w/w) of dry weight of the extract.

TABLE B
Illustrating the classification for secondary metabolites
as used herein. Please note that this illustration is not
to be construed as limiting. Other Class B plant secondary
metabolites may fall under a given class A class of plant
secondary metabolites. (Table splits across pages)

| Class A | Class B |
|---|---|
| alkaloids | piperine |
| alkaloids | theobromine |
| amino acids | amino acids |
| curcuminoids | diarylheptanoids |
| filling agent | filling agent |
| glycosides | flavonoid glycosides |
| glycosides | ginsenosides |
| glycosides | rosavins |
| glycosides | saponins |
| glycosides | triterpene glycosides |
| glycosides | triterpenoid saponins |
| polysaccharides | inulin |
| polyphenols | anthocyanidins |
| polyphenols | anthocyanin |
| polyphenols | apigenin flavone glycoside |
| polyphenols | flavanoid |
| polyphenols | flavones |
| polyphenols | flavanols |
| polyphenols | flavonoids |
| polyphenols | gingerol |
| polyphenols | phenolic acids |
| polyphenols | rosmarinic acid, hydroxycinnamic acid |
| polyphenols | vexitin |
| polysaccharides | beta glucan |
| polysaccharides | inulin |
| polysaccharides | pectin |
| terpenes | terpenoids, such as boswellic acids |
| terpenes | valerenic acid |
| terpenes | α-pinene |
| terpenes | triterpene glycosides, such as saponins |
| terpenes | steroids, such as triterpenoid withanolides |
| terpenes | triterpenoids, such as triterpenoid glycosides |
| vitamins | vitamin B12 |
| vitamins | vitamin B2 |
| vitamins | vitamin B6 |
| vitamins | vitamin C |
| vitamins | vitamin D3 |

Examples of preferred alkaloids are caffeine, huperzine A, minovincine, minovine, momordicin, sanjoinine A, vincaminorine, vincine, vincamine, withananine, piperine, theobromine and withanine. Examples of preferred flavonoids are catechin, epicatechin, epicatechin-3-gallate, isorhamnetin, kaempferol, quercetin, rutin, spinosin, swertish, xanthohumol, rosarin, rosavin, rosin, and cyanidin-3-O-glucoside. Preferred glycosides are sitoindosides IX, sitoindosides VII, sitoindosides VIII, sitoindosides X, charantin, momordin, and salidroside. Preferred phenolics are curcumin, macelignan, proanthocyanidins, ferulic acid, and rosmarinic acid. Preferred terpenes are erinacine A, erinacine B, erinacine C, a thujon, R-thujon, 1,8-cineole, camphor, carnosic acid, carnosol, manool, bilobalide, ginkgolide A, ginkgolide B, ginkgolide C, bacopaside I, bacopaside II, bacopaside N2, bacopaside X, bacoside A3, oleanolic acid, ursolic acid, boswellic acids, asiaticoside, asiatic acid, valeranone, valerenic acid, and madecassic acid. Frequent used vitamins in compositions or extracts may be vitamin A; B, such as B1, B2, B6, B7, B12; C; D, such D3; E and K. Most preferred are B, such as B2, B6, B12; C; D, such and D3. In one embodiment extract or composition can comprise 10 IU (or mg) and 800 IU (or mg) (e.g., between 100 IU (or mg) and 300 IU (or mg) or between 150 IU (or mg) and 250 IU (or mg)) of one or more vitamins. In one embodiment an extract or composition comprises between 0.006 mg and 2.5 mg (e.g., between 0.01 mg and 2.5 mg, between 0.1 mg and 2.5 mg, between 0.5 mg and 2.5 mg, between 0.006 mg and 1.5 mg, between 0.006 mg and 1.0 mg, or between 0.5 mg and 1.5 mg) of Vitamin B12. In some embodiments minerals may be added to the composition or extract. In some embodiments minerals may be added to the composition. In some embodiments, minerals may be independently selected from the group consisting of: Mg, Mn, Fe, Zn, Se, Cr, Cu and V.

Preferred amino acids may be selected from: essential amino acids, non-essential amino acids and proteinogenic amino acids. Essential amino acids may be histidine, isoleucine, leucine, lysine, methionine, phenylalanine, threonine, tryptophan, and valine. Non-essential amino acids may be alanine, arginine, asparagine, aspartic acid, cysteine, glutamic acid, glutamine, glycine, proline, serine, and tyrosine. The word “proteinogenic” means “protein creating”. Thus the term “proteinogenic amino acid” as used herein means amino acids which are incorporated biosynthetically into proteins during translation, which includes e.g. the aspartate family amino acid, erythrose 4-phosphate/phosphoenolpyruvate family amino acid, glutamine family amino acid, glycine, pyruvate family amino acid and serine family amino acid. Herein “proteinogenic amino acid” may include any one of the amino acids selected from the group consisting of: Arginine, Histidine, Lysine, Aspartic Acid, Glutamic Acid, Serine, Threonine, Asparagine, Glutamine, Cysteine, Selenocysteine, Glycine, Proline, Alanine, Isoleucine, Leucine, Methionine, Phenylalanine, Tryptophan, Tyrosine and Valine. Thus “proteinogenic amino acid” as used herein may also be any one of the amino acids selected from the group consisting of: N-formyl-L-methionine, L-alanine, L-arginine, L-asparagine, L-aspartic acid, L-cysteine, L-glutamic acid, L-glutamine, L-histidine, L-isoleucine, L-leucine, L-lysine, L-methionine, L-phenylalanine, L-proline, L-pyrrolysine, L-selenocysteine, L-serine, L-threonine, L-tryptophan, L-tyosine and L-valine.

Other preferred SMs are lupulone, theanine, valepotriates, hericerin A-E, iso-α-acids, bacopasaponin C, boswellic acids, elemicin, ginsenosides, humulone, hypericin, hyperforin, jujuboside A, B, madecassoside, mangiferin, myristicin, protopanaxadiols, protopanaxatriols, pseudohypericin, resveratrol, safrole, theanine, withaferine, and withanolides.

Thus “at least two” as used herein and referring to SMs, means that said SMs should not be selected from the same Class B genus. Thus both SMs can be selected within Class A, as long as they differ in their chemical name. However said SMs can also be selected from different Class A genus of SMs. Thus, if at least two secondary metabolites are chosen from the class of polyphenols, they should be selected from at least two Class B genus, such as gingerols and flavones.

The term “secondary metabolite”, “SMs” and “plant secondary metabolite” used herein may be used interchangeably and refer to SMs as described above.

The term “%” or “% (w/w)” as used throughout this application referring to content of e.g., SMs in an extract or composition, is meant the weight percentage of an ingredient by dry weight of e.g., a composition or extract. Thus, unless indicated otherwise any “%” or “% (w/w)” referring to concentration or content of an ingredient in a composition or extract is to be understood as indicating “by dry weight of” what the % (w/w) refers to. Further the % (w/w) as provided herein should be understood as the minimum content of said ingredient, when referring to SMs.

When using terms such as “about” and “approximately” in relation to numerical values the skilled person should immediately recognize that any effect or result, which may be associated with the given values can be obtained within a certain tolerance from the particular values. The term “about” as used herein thus means in reasonable vicinity of the stated numerical value, such as plus or minus 10% relative to the indicated number.

The term “extract” as used herein may be an extract from any biologic material. An extract or extracts disclosed herein may be part of compositions, which comprise said extract and additional pharmaceutically or nutritionally acceptable excipients, such as fillers, e.g., rice fillers, stabilizers, lubricants or other excipients known to be used in dietary supplement compositions. In preferred embodiments “extracts” as used herein are extracts derived from plants, yeast or fungi. Thus, the term “plant extract” refers to an extract from a plant, the term “yeast extract” refers to an extract derived from yest, such as from recombinant manufacturing of a particular plant secondary metabolite, and “fungi extract” refers to an extract from fungi. The term “extract” may be a mixture of several extracts, such as a mixture of a first plant extract and a second plant extract, a mixture of a plant extract and a fungi extract, or a plant extract and a yeast extract. Several extracts, means at least two extracts, such as two, three or more extracts, each from different sources, such as plant sources, fungi or yeast sources.

The “plant source” or “extract source” describing the extract from which the SM is retrieved and thus, which extract provides said SM to the extract comprising one or more extracts.

Herein, the term “dissolution” means the process of dissolving a solid substance into a solvent to make a solution. The term “disintegration”, “disintegrating”, “disintegrate” or “disintegrated” as used herein and when referring to a coating, is to be understood as said coating or composition being disintegrated into components, wherein some or all of the components are completely dissolved into the medium triggering said disintegration.

In the context of the present disclosure, an “acid resistant coating” should be understood as a coating that, when used to encapsulate the extract, at least in some cases allows for the encapsulated extract or part of the encapsulated extract to be delivered to the gut via an oral route without being subjected to and/or diluted in stomach acid. Hence, the acid resistant coating may be susceptible to chemical breakdown by stomach acid, as long as the acid resistant coating at least in some cases results in the encapsulated extract or part of the encapsulated extract not being subjected to stomach acid, when administered orally. For instance, the acid resistant coating may be a material that breaks down in stomach acid, but because of the acid resistant coating's thickness and/or structure, the average time it takes for the acid resistant coating to break down in stomach acid is longer than the average retention time in the stomach. For instance, the acid resistant coating may be one or more excipients of a tablet, the one or more excipients forming a thick layer enveloping the extract, the thick layer of excipients taking a longer time to break down in stomach acid, than the typical retention time in a human stomach. In another example, the acid resistant coating is the capsule material of a capsule, the capsule material having a slow rate of breakdown in stomach acid, such that at least in some cases when administered orally, the capsule material is still enveloping the extract when the capsule passes the stomach. The skilled person knows how to formulate tablets and capsules, such that at least some of the active ingredients (in this case extracts) will be released in the gut (i.e., have an acid resistant coating). This is also known in the art as delayed release formulations or late release formulations. Preferably, the acid resistant coating may have a slow degradation in acid or no degradation at all. Therefore, the coating may be a thin layer of a material that has no or slow degradation in acid.

It is contemplated in the current disclosure that the acid resistant coating may be a solution that increases the pH of the gastric juice, such as known from acid reflux therapy.

The term “encapsulated” as used herein means, that when something, i.e. the SMs and also the non-digestible plant fibre, when present, is “encapsulated”, it should be understood as being enveloped. For instance, the SMs may be enveloped by cellulose acetate, such that they are encapsulated by the cellulose acetate. In another example, the SMs may be formulated in a tablet comprising one or more excipients, such that one or more of the excipients encapsulates the plant secondary material in the tablet. In one embodiment said SMs are comprised in an extract and said extract is encapsulated. Said encapsulation may be in the form of a coating, a capsule or other material that provides resistant to the composition and extract at specified pH. However, the composition may comprise other ingredients, e.g., excipients, that are not encapsulated in the acid resistant coating. Thus, the coating may also be viewed as a shell or membrane, surrounding a core comprising one or more SMs as disclosed herein.

Thus, in on one embodiment a composition is in the form of a solid oral formulation. Thus, in on one embodiment a composition is in the form of a semi-solid oral formulation, wherein said semi-solid oral formulation is a gel, gelatine or fruit gum. In one embodiment a composition can, be formulated in a liquid, solution, suspension, tablet, powder, cream, mist, atomized vapor, aerosol, soft gelatine capsule, hard gelatine capsule, a gel, a confectionary, a shake, a bar, or a supplemented food. In one embodiment a pharmaceutical composition according to the present invention is manufactured into a tablet. In one embodiment a composition is manufactured into a tablet for oral administration. In one embodiment the capsule in which a composition is provided in is selected from the group of capsules known to the person skilled in the art.

In one embodiment the capsule in which a composition is provided in is selected from the group of capsules comprising gel, gelatine or gelatine-like material.

In one embodiment the capsule in which a composition is provided in is selected from the group of capsules: fish-gelatin, HMPC, pullan, procine gelatin.

The term “microencapsulation” and “nanoencapsulation” as used herein are coating of various substances within another material at sizes on the nano or micro scale. The encapsulation technologies are well known to the skilled person, and concerns techniques, wherein particles or droplets are surrounded by a coating, providing a small sphere, with a near-uniform wall enclosing the encapsulated material. The diameter of the sphere's pores are between about 1-2 nm to about 2-3 μm and may be determined by electron microscopy imaging. The encapsulated material, herein the at SMs or extract may be seen as the “core” or “internal phase”, whereas encapsulation may be seen as the “shell”, “coating”, or “membrane”. In some cases, materials like lipids and polymers, such as alginate, may be used as a mixture to trap the material of interest inside the encapsulation. Said coating may be selected from all suitable coatings, however commonly used coatings are ethyl cellulose, polyvinyl alcohol, gelatine or sodium alginate. Several methods to apply microencapsulations are applicable and known to those skilled in the art, however examples of suitable methods are: pan coating, centrifugal extrusion, virational nozzle, spray-dying, spray chilling and spray freeze drying, ionotropic gelation, coacervation-phase separation, interfacial polycondensation or cross-linking, in situ polymerization and matrix polymerization.

### EXAMPLES

### Example 1—Selected Compositions According to the Present Invention, Increasing Bacteria Abundancy

Experiments were carried out both in vitro (colon fermentation model, FIGS. 1)-4) and in vivo (human volunteers, FIG. 5).

For in vitro studies, formulations were added to added to faecal samples from healthy donors and investigated in a fermentation process simulating the human colon by means of controlling temperature (36.5° C.), anaerobic conditions, and pH (between 6-7) and a dedicated basal medium according to Wiese et al 2018 (DOI 10.7717/peerj.4268). The experiments were carried out for 24 hours in triplicates. Samples were analysed before and after fermentation, and in comparison, to a control run without any addition of a formulation. For in vivo studies, formulations were administered to healthy volunteers by means of capsules size 0 with a dose of 2 capsules per day for a period of 15 days. Faecal samples were taken before and after treatment and stabilized in an analytical buffer protecting nucleic acids prior to sample processing.

Samples were analysed for abundance of different bacteria species and genera by following a protocol for DNA isolation, gene library preparation, high through-put sequencing and data treatment (both 16S RNA and whole genome sequencing) and statistical data analysis using methodologies as pair-wise comparison, analysis of variance (ANOVA), principal component analysis (PCA), among others.

Table 1—summarizes the results of the above experiments.

TABLE 1

| | | | | fold |
|---|---|---|---|---|
| bacteria | Formula | Control | Treatment | change |
| [genus] | [ID] | [abundance %] | [abundance %] | [%] |
| Bifidobacterium | NPM-2 | 10.30 | 16.10 | 56 |
| | MEM-7 | 6.70 | 19.50 | 191 |
| | MM-11 | 7.10 | 41.50 | 485 |
| Bacteroides | NPM-2 | 3.70 | 6.50 | 76 |
| | NPM-1 | 3.60 | 11.30 | 214 |
| Faecalibacterium | NPM-2 | 0.50 | 3.40 | 580 |
| | MEM-7 | 1.10 | 3.60 | 227 |
| | NPM-1 | 1.90 | 10.10 | 432 |
| Prevotella | NPM-2 | 0.01 | 1.30 | 18471 |
| | MEM-3 | 0.01 | 0.94 | 13329 |
| | NPM-1 | 0.01 | 1.43 | 28500 |
| Blautia | FM-01 | 2.07 | 1.20 | −42 |
| | FM-01 | 3.27 | 1.81 | −45 |
| | FM-01 | 7.20 | 4.48 | −38 |

### Conclusion Example 1

The FIGS. 1-4 collectively show that faecal samples after treatment with compositions consisting of extracts containing two or more SMs, the SMs constituting at least 10% (w/w) by dry weight of the extract, significantly increases in the abundance of beneficial bacteria in the faecal samples. Furthermore, FIG. 5 shows that faecal samples after treatment with a composition consisting of extracts containing two or more SMs, the SMs constituting at least 10% (w/w) by dry weight of the extract, significantly reduces the abundance of Blautia in the faecal samples, a genus of bacteria associated with negative health impacts.

### Example 2a—Secondary Metabolites have an Increasing Effect, i.e., are Positively Correlated to Increase of Bacteria Abundancy

The compositions specified in table A were tested in 8 human faeces donors and analysed in accordance with the described in vitro method in example 1. Data was compiled from a representative selection of at least two human faeces donors and normalised to the controls for each experiment. The total abundance values were normalised to Log 2 fold changes and the correlation between the secondary metabolites used in said formulation was calculated based on the normalised Log 2 fold change relative to the control for each experiment.

Secondary Metabolites Correlated with Increased Bacteria Abundancy:

The below lists a selection of SMs which show a correlation factor of above 0.5 for increasing one or more specific bacteria abundancy. A series of other plant secondary metabolites also correlated with bacteria abundancy, however only those with a correlation factor above 0.5 are shown here. SMs with a correlation factor above 0.5 for increasing specific bacteria abundancy may be selected from the group consisting of: anthocyanidins, withanolides, flavonoids, triterpenoid saponins, beta glucan, saponins, proanthocyanidins, α-pinene, boswellic acids, theobromine, diarylheptanoids, phenolic acids, piperine, flavones, ginsenosides, saponins, gingerol, flavonoid glycosides and pectins. Thus, all coming from the class A categorizations of polyphenols, triterpenoids, glycosides, polysaccharides, terpenes, alkaloids, curcuminoids and polysaccharides.

The below table illustrates secondary metabolite from different extract sources and the correlation factor between secondary metabolite and Bacteria abundance.

TABLE 2
SMs correlated with increase specific Bacteria with a positive correlation factor of 0.5
or more. The table further indicates the extract source. (Table splits across pages)

| | Secondary | Secondary | | |
|---|---|---|---|---|
| Bacteria | Metabolite | Metabolite | Correlation | |
| [genus] | Class A | Class B | Factor | Extract Source |
| Akkermansia | polyphenols | anthocyanidins | 0.52 | vaccinium myrtillus |
| Akkermansia | terpenes | withanolides | 0.66 | withania somnifera |
| Bacteroides | polyphenols | flavonoids | 0.50 | camellia sinensis |
| Bacteroides | polyphenols | flavonoids | 0.55 | humulus hupulus |
| Bacteroides | glycosides | triterpenoid saponins | 0.56 | bacopa monnieri |
| Sutterella | polysaccharides | beta glucan | 1.00 | hordeum vulgare |
| Bacteroides | terpenes | saponins | 0.85 | centella asiatica |
| Bacteroides | polyphenols | flavonoids | 0.95 | ziziphus jujuba |
| Bacteroides | polyphenols | flavonoids | 0.99 | gingko biloba |
| Bacteroides | polyphenols | proanthocyanidins | 1.00 | vitis vinifera |
| Bacteroides | terpenes | saponins | 1.00 | centella asiatica |
| Bacteroides | terpenes | withanolides | 1.00 | withania somnifera |
| Bacteroides | terpenes | α-pinene | 1.00 | salvia officinalis L |
| Bacteroides | polyphenols | anthocyanidins | 1.00 | vaccinium myrtillus |
| Bifidobacterium | glycosides | triterpenoid saponins | 0.53 | bacopa monnieri |
| Veillonella | polysaccharides | beta glucan | 1.00 | hericium erinaceus |
| Bifidobacterium | terpenes | withanolides | 0.64 | withania somnifera |
| Bifidobacterium | terpenes | α-pinene | 0.64 | salvia officinalis L |
| Parabacteroides | terpenes | boswellic acids | 0.56 | boswellia serrata |
| Bifidobacterium | terpenes | withanolides | 0.70 | withania somnifera |
| Bifidobacterium | alkaloids | theobromine | 0.79 | theobroma cacao |
| Ruminococcus | terpenes | boswellic acids | 0.50 | boswellia serrata |
| Bifidobacterium | polyphenols | flavonoids | 0.88 | gingko biloba |
| Bifidobacterium | polyphenols | anthocyanidins | 1.00 | vaccinium myrtillus |
| Blautia | curcuminoids | diarylheptanoids | 0.70 | tumeric rhizome |
| Blautia | polyphenols | proanthocyanidins | 0.78 | vitis vinifera |
| Blautia | terpenes | saponins | 0.78 | centella asiatica |
| Blautia | polyphenols | phenolic acids | 0.86 | citrus sinensis |
| Blautia | polyphenols | flavonoids | 0.92 | ziziphus jujuba |
| Blautia | polyphenols | anthocyanidins | 1.00 | vaccinium myrtillus |
| Clostridium | alkaloids | piperine | 0.56 | piper nigrum |
| Clostridium | polyphenols | proanthocyanidins | 0.59 | vitis vinifera |
| Clostridium | curcuminoids | diarylheptanoids | 0.62 | tumeric rhizome |
| Clostridium | alkaloids | theobromine | 0.68 | theobroma cacao |
| Clostridium | polyphenols | flavonoids | 0.71 | citrus paradisi |
| Clostridium | curcuminoids | diarylheptanoids | 0.72 | curcuma longa |
| Clostridium | polyphenols | flavonoids | 0.86 | camellia sinensis |
| Clostridium | terpenes | withanolides | 0.86 | withania somnifera |
| Clostridium | terpenes | saponins | 0.87 | centella asiatica |
| Clostridium | polyphenols | proanthocyanidins | 0.92 | vitis vinifera |
| Clostridium | terpenes | saponins | 0.92 | centella asiatica |
| Veillonella | terpenes | boswellic acids | 0.82 | boswellia serrata |
| Collinsella | polyphenols | flavonoids | 0.71 | ziziphus jujuba |
| Collinsella | polyphenols | proanthocyanidins | 0.75 | vitis vinifera |
| Collinsella | terpenes | saponins | 0.75 | centella asiatica |
| Bacteroides | terpenes | boswellic acids | 0.78 | boswellia serrata |
| Coprococcus | polyphenols | flavones | 0.66 | gingko biloba |
| Coprococcus | polyphenols | flavonoids | 0.81 | camellia sinensis |
| Coprococcus | polyphenols | flavonoids | 0.82 | gingko biloba |
| Coprococcus | polyphenols | proanthocyanidins | 0.97 | vitis vinifera |
| Coprococcus | terpenes | saponins | 0.97 | centella asiatica |
| Coprococcus | glycosides | ginsenosides | 0.97 | panax ginseng |
| Bifidobacterium | terpenes | boswellic acids | 0.54 | boswellia serrata |
| Dialister | curcuminoids | diarylheptanoids | 0.71 | curcuma longa |
| Roseburia | terpenes | boswellic acids | 0.98 | boswellia serrata |
| Dorea | terpenes | withanolides | 0.86 | withania somnifera |
| Dorea | terpenes | α-pinene | 0.86 | salvia officinalis L |
| Dorea | polyphenols | flavonoids | 0.98 | gingko biloba |
| Dorea | polyphenols | anthocyanidins | 1.00 | vaccinium myrtillus |
| Enterobacter | polyphenols | flavonoids | 0.50 | citrus paradisi |
| Sutterella | terpenes | boswellic acids | 0.55 | boswellia serrata |
| Enterobacter | alkaloids | theobromine | 0.71 | theobroma cacao |
| Enterobacter | polyphenols | flavonoids | 0.71 | camellia sinensis |
| Enterobacter | curcuminoids | diarylheptanoids | 0.71 | curcuma longa |
| Escherichia | alkaloids | piperine | 0.55 | piper nigrum |
| Escherichia | polyphenols | proanthocyanidins | 0.56 | vitis vinifera |
| Escherichia | glycosides | saponins | 0.61 | Momordica Charantia |
| Escherichia | polyphenols | flavonoids | 0.61 | camellia sinensis |
| Escherichia | terpenes | saponins | 0.74 | centella asiatica |
| Escherichia | alkaloids | theobromine | 0.76 | theobroma cacao |
| Escherichia | terpenes | withanolides | 0.80 | withania somnifera |
| Escherichia | terpenes | α-pinene | 0.80 | salvia officinalis L |
| Escherichia | polyphenols | flavonoids | 0.89 | citrus paradisi |
| Bifidobacterium | polysaccharides | beta glucan | 0.85 | hericium erinaceus |
| Eubacterium | polyphenols | phenolic acids | 0.56 | citrus sinensis |
| Eubacterium | polyphenols | flavonoids | 0.58 | citrus paradisi |
| Eubacterium | polyphenols | flavonoids | 0.67 | camellia sinensis |
| Eubacterium | polyphenols | flavonoids | 0.70 | humulus hupulus |
| Eubacterium | polyphenols | flavonoids | 0.94 | gingko biloba |
| Eubacterium | polyphenols | proanthocyanidins | 0.95 | vitis vinifera |
| Eubacterium | terpenes | saponins | 0.95 | centella asiatica |
| Eubacterium | curcuminoids | diarylheptanoids | 0.96 | tumeric rhizome |
| Dialister | polysaccharides | beta glucan | 1.00 | hericium erinaceus |
| Faecalibacterium | polyphenols | anthocyanidins | 0.62 | vaccinium myrtillus |
| Faecalibacterium | polyphenols | gingerol | 0.74 | zingiber officinale |
| Faecalibacterium | polyphenols | flavonoids | 0.81 | gingko biloba |
| Faecalibacterium | polysaccharides | beta glucan | 1.00 | hericium erinaceus |
| Lactobacillus | polysaccharides | beta glucan | 1.00 | hericium erinaceus |
| Holdemania | terpenes | saponins | 0.83 | centella asiatica |
| Lactobacillus | polyphenols | flavonoids | 0.86 | camellia sinensis |
| Lactobacillus | polyphenols | flavonoids | 0.92 | ziziphus jujuba |
| Lactobacillus | terpenes | saponins | 0.99 | centella asiatica |
| Lactobacillus | glycosides | ginsenosides | 0.99 | panax ginseng |
| Prevotella | polysaccharides | beta glucan | 1.00 | hericium erinaceus |
| Ruminococcus | polysaccharides | beta glucan | 0.52 | hericium erinaceus |
| Slackia | polysaccharides | beta glucan | 1.00 | hericium erinaceus |
| Parabacteroides | polyphenols | flavonoids | 0.57 | gingko biloba |
| Parabacteroides | polyphenols | proanthocyanidins | 0.65 | vitis vinifera |
| Parabacteroides | terpenes | saponins | 0.65 | centella asiatica |
| Parabacteroides | curcuminoids | diarylheptanoids | 0.69 | tumeric rhizome |
| Parabacteroides | polyphenols | flavonoids | 0.77 | humulus hupulus |
| Parabacteroides | polyphenols | flavones | 0.78 | gingko biloba |
| Parabacteroides | glycosides | ginsenosides | 0.98 | panax ginseng |
| Clostridium | polysaccharides | beta glucan | 1.00 | hordeum vulgare |
| Parabacteroides | polyphenols | anthocyanidins | 1.00 | vaccinium myrtillus |
| Prevotella | polyphenols | flavones | 0.90 | gingko biloba |
| Prevotella | polyphenols | flavonoids | 0.92 | ziziphus jujuba |
| Collinsella | polysaccharides | beta glucan | 1.00 | hordeum vulgare |
| Coprococcus | polysaccharides | beta glucan | 1.00 | hordeum vulgare |
| Prevotella | glycosides | ginsenosides | 1.00 | panax ginseng |
| Roseburia | polyphenols | proanthocyanidins | 0.70 | vitis vinifera |
| Roseburia | terpenes | saponins | 0.70 | centella asiatica |
| Roseburia | polyphenols | flavonoids | 0.75 | ziziphus jujuba |
| Roseburia | polyphenols | flavonoids | 0.85 | gingko biloba |
| Roseburia | terpenes | withanolides | 0.86 | withania somnifera |
| Roseburia | terpenes | α-pinene | 0.86 | salvia officinalis L |
| Escherichia | polysaccharides | beta glucan | 1.00 | hordeum vulgare |
| Faecalibacterium | polysaccharides | beta glucan | 1.00 | hordeum vulgare |
| Lactobacillus | polysaccharides | beta glucan | 1.00 | hordeum vulgare |
| Prevotella | polysaccharides | beta glucan | 1.00 | hordeum vulgare |
| Roseburia | polysaccharides | beta glucan | 1.00 | hordeum vulgare |
| Ruminococcus | polyphenols | flavones | 0.75 | gingko biloba |
| Ruminococcus | glycosides | ginsenosides | 0.83 | panax ginseng |
| Ruminococcus | polyphenols | phenolic acids | 0.90 | citrus sinensis |
| Ruminococcus | polyphenols | flavonoids | 0.95 | gingko biloba |
| Ruminococcus | polyphenols | proanthocyanidins | 0.97 | vitis vinifera |
| Ruminococcus | terpenes | saponins | 0.97 | centella asiatica |
| Ruminococcus | polysaccharides | beta glucan | 1.00 | hordeum vulgare |
| Slackia | curcuminoids | diarylheptanoids | 0.80 | curcuma longa |
| Bifidobacterium | glycosides | flavonoid glycosides | 0.69 | fagopyrum esculentum |
| Streptococcus | polyphenols | flavonoids | 0.56 | camellia sinensis |
| Streptococcus | terpenes | withanolides | 0.57 | withania somnifera |
| Streptococcus | terpenes | α-pinene | 0.57 | salvia officinalis L |
| Streptococcus | polyphenols | phenolic acids | 0.82 | citrus sinensis |
| Streptococcus | curcuminoids | diarylheptanoids | 0.88 | tumeric rhizome |
| Streptococcus | polyphenols | proanthocyanidins | 0.93 | vitis vinifera |
| Streptococcus | terpenes | saponins | 0.93 | centella asiatica |
| Streptococcus | polyphenols | flavonoids | 0.94 | ziziphus jujuba |
| Sutterella | polyphenols | phenolic acids | 0.54 | citrus sinensis |
| Enterobacter | glycosides | flavonoid glycosides | 0.52 | fagopyrum esculentum |
| Faecalibacterium | glycosides | flavonoid glycosides | 0.56 | fagopyrum esculentum |
| Veillonella | polysaccharides | pectin | 0.54 | citrus limon |
| Veillonella | curcuminoids | diarylheptanoids | 0.56 | tumeric rhizome |
| Veillonella | polyphenols | phenolic acids | 0.56 | citrus sinensis |
| Veillonella | polyphenols | gingerol | 0.56 | zingiber officinale |
| Parabacteroides | glycosides | flavonoid glycosides | 1.00 | fagopyrum esculentum |
| Roseburia | glycosides | flavonoid glycosides | 0.88 | fagopyrum esculentum |

End of Table 2—SMs correlated with increase specific Bacteria with a positive correlation factor of 0.5 or more. The table further indicates the extract source.

From the above it is evident that SMs from different sources, such as different plant extract sources may be used to increase specific bacteria abundancies. Some secondary metabolites, from selected extract sources may be more potent than others for specific bacteria and thus provide flexibility in selection and dose, dependent on which effect is desired. The following table illustrates where correlation was particularly pronounced, i.e., a correlation factor of 0.95 and above to specific bacteria abundancies for α-pinene, beta glucan, anthocyanidins, proanthocyanidins, saponins, ginsenosides, boswellic acids, diarylheptanoids and flavonoids, thus from the class A SMs categories: terpenes, polysaccharides, polyphenols, glycosides, terpenes and curcuminoids.

TABLE 3
SMs correlated with increase specific Bacteria with a correlation factor of 0.95 or
more for increasing at least one Bacteria abundancy. (The table splits across pages).

| | Secondary | Secondary | | |
|---|---|---|---|---|
| Bacteria | Metabolite | Metabolite | Correlation | |
| [genus] | Class A | Class B | Factor | Extract Source |
| Sutterella | polysaccharides | beta glucan | 1.00 | hordeum vulgare |
| Bacteroides | polyphenols | flavonoids | 0.99 | gingko biloba |
| Bacteroides | polyphenols | proanthocyanidins | 1.00 | vitis vinifera |
| Bacteroides | terpenes | saponins | 1.00 | centella asiatica |
| Bacteroides | terpenes | withanolides | 1.00 | withania somnifera |
| Bacteroides | terpenes | α-pinene | 1.00 | salvia officinalis L |
| Bacteroides | polyphenols | anthocyanidins | 1.00 | vaccinium myrtillus |
| Veillonella | polysaccharides | beta glucan | 1.00 | hericium erinaceus |
| Bifidobacterium | polyphenols | anthocyanidins | 1.00 | vaccinium myrtillus |
| Blautia | polyphenols | anthocyanidins | 1.00 | vaccinium myrtillus |
| Coprococcus | polyphenols | proanthocyanidins | 0.97 | vitis vinifera |
| Coprococcus | terpenes | saponins | 0.97 | centella asiatica |
| Coprococcus | glycosides | ginsenosides | 0.97 | panax ginseng |
| Roseburia | terpenes | boswellic acids | 0.98 | boswellia serrata |
| Dorea | polyphenols | flavonoids | 0.98 | gingko biloba |
| Dorea | polyphenols | anthocyanidins | 1.00 | vaccinium myrtillus |
| Eubacterium | polyphenols | proanthocyanidins | 0.95 | vitis vinifera |
| Eubacterium | terpenes | saponins | 0.95 | centella asiatica |
| Eubacterium | curcuminoids | diarylheptanoids | 0.96 | tumeric rhizome |
| Dialister | polysaccharides | beta glucan | 1.00 | hericium erinaceus |
| Faecali-bacterium | polysaccharides | beta glucan | 1.00 | hericium erinaceus |
| Lactobacillus | polysaccharides | beta glucan | 1.00 | hericium erinaceus |
| Lactobacillus | terpenes | saponins | 0.99 | centella asiatica |
| Lactobacillus | glycosides | ginsenosides | 0.99 | panax ginseng |
| Prevotella | polysaccharides | beta glucan | 1.00 | hericium erinaceus |
| Slackia | polysaccharides | beta glucan | 1.00 | hericium erinaceus |
| Para-bacteroides | glycosides | ginsenosides | 0.98 | panax ginseng |
| Clostridium | polysaccharides | beta glucan | 1.00 | hordeum vulgare |
| Para-bacteroides | polyphenols | anthocyanidins | 1.00 | vaccinium myrtillus |
| Collinsella | polysaccharides | beta glucan | 1.00 | hordeum vulgare |
| Coprococcus | polysaccharides | beta glucan | 1.00 | hordeum vulgare |
| Prevotella | glycosides | ginsenosides | 1.00 | panax ginseng |
| Escherichia | polysaccharides | beta glucan | 1.00 | hordeum vulgare |
| Faecali-bacterium | polysaccharides | beta glucan | 1.00 | hordeum vulgare |
| Lactobacillus | polysaccharides | beta glucan | 1.00 | hordeum vulgare |
| Prevotella | polysaccharides | beta glucan | 1.00 | hordeum vulgare |
| Roseburia | polysaccharides | beta glucan | 1.00 | hordeum vulgare |
| Ruminococcus | polyphenols | flavonoids | 0.95 | gingko biloba |
| Ruminococcus | polyphenols | pro-anthocyanidins | 0.97 | vitis vinifera |
| Ruminococcus | terpenes | saponins | 0.97 | centella asiatica |
| Ruminococcus | polysaccharides | beta glucan | 1.00 | hordeum vulgare |
| Para-bacteroides | glycosides | flavonoid glycosides | 1.00 | fagopyrum esculentum |

End of Table 3—SMs correlated with increase specific Bacteria with a correlation factor of 0.95 and more for more than one Bacteria.

The following table illustrates that some of the SMs are particularly useful if a combined increase of more than one bacteria abundancy is desired.

TABLE 4
Secondary metabolites having a. positive correlation factor of
at least 0.5 for increase in more than one Bacteria abundancy.

| Bacteria [genus] combination | SM Class B |
|---|---|
| Akkermansia-Bacteroides-Bifidobacterium | withanolides |
| | anthocyanidins |
| Bacteroides-Bifidobacterium | α-pinene |
| | saponins |
| | flavonoids |
| | boswellic |
| Clostridium-Coprococcus-Escherichia | Beta glucan |
| | Anthocyanidina |
| | saponins |
| | flavonoids |
| Clostridium-Escherichia | withanolides |
| | flavanones |
| | theobromine |
| | piperine |
| Eubacterium-Faecalibacterium-Lactobacillus | flavonoids |
| Eubacterium-Faecalibacterium | anthocyanidins |
| Eubacterium-Lactobacillus | saponins |
| Roseburia-Ruminococcus-Streptococcus | beta-glucan |
| | flavonoids |
| | anthocyanidins |
| | saponins |
| Roseburia-Ruminococcus | boswellic |
| | ginsenosides |
| Roseburia-Streptococcus | withanolides |
| | α-pinene |

Table 4—Secondary metabolites having a positive correlation factor of at least 0.5 for increase in more than one Bacteria.

### Example 2b—Secondary Metabolites Decreasing Effect. i.e. Negatively Correlated to Increase of Bacteria Abundancy. i.e. Reducing Bacteria Abundancy

Secondary Metabolites Correlated with Decreased Bacteria Abundancy:

Secondary metabolites specified by their class B categorizations with a correlation factor below −0.5 for increasing specific bacteria abundancy may be selected from the group consisting of: flavonoids, beta glucan, ginsenosides, flavones, theobromine, saponins, proanthocyanidins, diarylheptanoids, gingerol, boswellic acids, flavonoid glycosides, phenolic acids, saponins, piperine, anthocyanidins, α-pinene, withanolides, rosavins and inulin can reduce the bacteria abundancy of specific bacteria, these are from the SM class A catergories: class A secondary metabolite group consisting of: polyphenols, polysaccharides, terpenes, alkaloids, glycosides, terpenes and curcuminoids.

The below table indicates the bacteria abundancies affected by said SMs with a correlation factor of lower than −0.5 below. A series of other SMs are also negatively correlated with bacteria abundancy, however not shown here. From the below it is evident that SMs for decreasing specific bacteria abundancies in the gut can be selected from the class A secondary metabolite group consisting of: polyphenols, polysaccharides, terpenes, alkaloids, glycosides, terpenes and curcuminoids.

The below table also illustrates secondary metabolites from different extract sources and the correlation factor between secondary metabolite and bacteria genus abundance.

TABLE 5
SMs with a correlation factor of below −0.5 for more than one Bacteria. The
table further indicates suitable Extract Sources. (Table splits across pages)

| | Secondary | Secondary | | |
|---|---|---|---|---|
| Bacteria | Metabolite | Metabolite | Correlation | |
| [genus] | Class A | Class B | Factor | Extract Source |
| Akkermansia | polyphenols | flavonoids | −1.00 | camellia sinensis |
| Bacteroides | polysaccharides | beta glucan | −1.00 | hordeum vulgare |
| Bacteroides | glycosides | ginsenosides | −1.00 | panax ginseng |
| Bacteroides | polyphenols | flavones | −1.00 | gingko biloba |
| Bacteroides | alkaloids | theobromine | −1.00 | theobroma cacao |
| Bacteroides | terpenes | saponins | −1.00 | centella asiatica |
| Bacteroides | polyphenols | anthocyanidins | −1.00 | vitis vinifera |
| Bacteroides | curcuminoids | diarylheptanoids | −1.00 | curcuma longa |
| Bacteroides | polyphenols | gingerol | −1.00 | zingiber officinale |
| Bacteroides | terpenes | boswellic acids | −1.00 | boswellia serrata |
| Bacteroides | polysaccharides | beta glucan | −1.00 | hericium erinaceus |
| Bacteroides | polyphenols | flavonoids | −1.00 | citrus paradisi |
| Bacteroides | glycosides | flavonoid glycosides | −1.00 | fagopyrum esculentum |
| Bacteroides | polyphenols | flavonoids | −1.00 | camellia sinensis |
| Bacteroides | curcuminoids | diarylheptanoids | −1.00 | tumeric rhizome |
| Bifidobacterium | polysaccharides | beta glucan | −1.00 | hordeum vulgare |
| Bifidobacterium | terpenes | boswellic acids | −1.00 | boswellia serrata |
| Bifidobacterium | polyphenols | flavonoids | −1.00 | ziziphus jujuba |
| Bifidobacterium | polyphenols | phenolic acids | −0.99 | citrus sinensis |
| Bifidobacterium | terpenes | saponins | −0.98 | centella asiatica |
| Bifidobacterium | polyphenols | anthocyanidins | −0.98 | vitis vinifera |
| Bifidobacterium | polyphenols | flavonoids | −0.97 | camellia sinensis |
| Bifidobacterium | glycosides | ginsenosides | −0.97 | panax ginseng |
| Bifidobacterium | curcuminoids | diarylheptanoids | −0.97 | tumeric rhizome |
| Bifidobacterium | curcuminoids | diarylheptanoids | −0.97 | curcuma longa |
| Blautia | polysaccharides | beta glucan | −0.96 | hordeum vulgare |
| Blautia | alkaloids | theobromine | −0.96 | theobroma cacao |
| Blautia | polyphenols | flavonoids | −0.96 | citrus paradisi |
| Blautia | glycosides | saponins | −0.94 | Momordica Charantia |
| Blautia | alkaloids | piperine | −0.90 | piper nigrum |
| Blautia | terpenes | saponins | −0.90 | centella asiatica |
| Blautia | glycosides | flavonoid glycosides | −0.90 | fagopyrum esculentum |
| Blautia | glycosides | ginsenosides | −0.89 | panax ginseng |
| Blautia | polyphenols | gingerol | −0.89 | zingiber officinale |
| Blautia | polyphenols | anthocyanidins | −0.88 | vitis vinifera |
| Blautia | terpenes | boswellic acids | −0.88 | boswellia serrata |
| Clostridium | polyphenols | anthocyanidins | −0.88 | vaccinium myrtillus |
| Clostridium | polyphenols | gingerol | −0.87 | zingiber officinale |
| Clostridium | polyphenols | flavones | −0.87 | gingko biloba |
| Collinsella | polyphenols | anthocyanidins | −0.87 | vaccinium myrtillus |
| Collinsella | polyphenols | flavonoids | −0.87 | camellia sinensis |
| Collinsella | terpenes | boswellic acids | −0.87 | boswellia serrata |
| Collinsella | glycosides | ginsenosides | −0.85 | panax ginseng |
| Collinsella | polyphenols | anthocyanidins | −0.85 | vitis vinifera |
| Collinsella | alkaloids | theobromine | −0.84 | theobroma cacao |
| Collinsella | polyphenols | flavones | −0.84 | gingko biloba |
| Collinsella | polyphenols | phenolic acids | −0.82 | citrus sinensis |
| Collinsella | glycosides | flavonoid glycosides | −0.82 | fagopyrum esculentum |
| Coprococcus | polyphenols | anthocyanidins | −0.82 | vaccinium myrtillus |
| Coprococcus | polysaccharides | beta glucan | −0.82 | hericium erinaceus |
| Coprococcus | terpenes | α-pinene | −0.81 | salvia officinalis L |
| Coprococcus | terpenes | withanolides | −0.81 | withania somnifera |
| Coprococcus | glycosides | flavonoid glycosides | −0.81 | fagopyrum esculentum |
| Coprococcus | polyphenols | flavonoids | −0.81 | citrus paradisi |
| Coprococcus | polyphenols | flavonoids | −0.80 | ziziphus jujuba |
| Coprococcus | alkaloids | theobromine | −0.80 | theobroma cacao |
| Coprococcus | glycosides | rosavins | −0.78 | rhodiola rosea |
| Coprococcus | glycosides | saponins | −0.78 | Momordica Charantia |
| Coprococcus | polysaccharides | inulin | −0.78 | taraxacum officinale |
| Coprococcus | alkaloids | piperine | −0.78 | piper nigrum |
| Dialister | glycosides | ginsenosides | −0.78 | panax ginseng |
| Dialister | polyphenols | flavones | −0.77 | gingko biloba |
| Dialister | terpenes | saponins | −0.76 | centella asiatica |
| Dialister | alkaloids | theobromine | −0.75 | theobroma cacao |
| Dialister | glycosides | ginsenosides | −0.74 | panax ginseng |
| Dorea | polysaccharides | beta glucan | −0.73 | hordeum vulgare |
| Dorea | polysaccharides | beta glucan | −0.73 | hericium erinaceus |
| Dorea | glycosides | ginsenosides | −0.73 | panax ginseng |
| Dorea | polyphenols | flavonoids | −0.72 | camellia sinensis |
| Dorea | terpenes | saponins | −0.72 | centella asiatica |
| Dorea | polyphenols | anthocyanidins | −0.71 | vitis vinifera |
| Dorea | polyphenols | flavonoids | −0.71 | citrus paradisi |
| Dorea | polyphenols | flavones | −0.71 | gingko biloba |
| Dorea | polysaccharides | inulin | −0.71 | taraxacum officinale |
| Dorea | terpenes | saponins | −0.71 | centella asiatica |
| Dorea | alkaloids | theobromine | −0.70 | theobroma cacao |
| Dorea | glycosides | rosavins | −0.70 | rhodiola rosea |
| Dorea | terpenes | saponins | −0.70 | centella asiatica |
| Eubacterium | terpenes | α-pinene | −0.70 | salvia officinalis L |
| Eubacterium | terpenes | withanolides | −0.69 | withania somnifera |
| Eubacterium | polyphenols | flavonoids | −0.68 | ziziphus jujuba |
| Eubacterium | polyphenols | gingerol | −0.68 | zingiber officinale |
| Eubacterium | terpenes | saponins | −0.68 | centella asiatica |
| Faecalibacterium | polyphenols | anthocyanidins | −0.68 | vaccinium myrtillus |
| Faecalibacterium | terpenes | saponins | −0.67 | centella asiatica |
| Faecalibacterium | polyphenols | anthocyanidins | −0.67 | vitis vinifera |
| Faecalibacterium | glycosides | ginsenosides | −0.67 | panax ginseng |
| Faecalibacterium | polyphenols | flavonoids | −0.67 | citrus paradisi |
| Faecalibacterium | terpenes | α-pinene | −0.67 | salvia officinalis L |
| Faecalibacterium | terpenes | withanolides | −0.67 | withania somnifera |
| Faecalibacterium | alkaloids | theobromine | −0.67 | theobroma cacao |
| Faecalibacterium | polyphenols | flavonoids | −0.66 | camellia sinensis |
| Faecalibacterium | curcuminoids | diarylheptanoids | −0.66 | tumeric rhizome |
| Faecalibacterium | alkaloids | piperine | −0.66 | piper nigrum |
| Faecalibacterium | terpenes | saponins | −0.66 | centella asiatica |
| Faecalibacterium | glycosides | saponins | −0.66 | Momordica Charantia |
| Faecalibacterium | terpenes | withanolides | −0.66 | withania somnifera |
| Lactobacillus | polyphenols | anthocyanidins | −0.66 | vaccinium myrtillus |
| Lactobacillus | polyphenols | flavonoids | −0.65 | humulus hupulus |
| Lactobacillus | polyphenols | flavonoids | −0.65 | citrus paradisi |
| Parabacteroides | polysaccharides | beta glucan | −0.65 | hordeum vulgare |
| Parabacteroides | polysaccharides | beta glucan | −0.65 | hericium erinaceus |
| Parabacteroides | curcuminoids | diarylheptanoids | −0.65 | curcuma longa |
| Parabacteroides | alkaloids | theobromine | −0.64 | theobroma cacao |
| Parabacteroides | polyphenols | flavonoids | −0.64 | ziziphus jujuba |
| Parabacteroides | polyphenols | anthocyanidins | −0.64 | vitis vinifera |
| Parabacteroides | glycosides | saponins | −0.64 | Momordica Charantia |
| Prevotella | polyphenols | anthocyanidins | −0.63 | vaccinium myrtillus |
| Prevotella | terpenes | boswellic acids | −0.63 | boswellia serrata |
| Prevotella | curcuminoids | diarylheptanoids | −0.63 | curcuma longa |
| Prevotella | polyphenols | flavonoids | −0.61 | gingko biloba |
| Prevotella | polyphenols | anthocyanidins | −0.61 | vitis vinifera |
| Prevotella | polyphenols | flavonoids | −0.61 | humulus hupulus |
| Prevotella | polyphenols | anthocyanidins | −0.61 | vitis vinifera |
| Roseburia | polyphenols | anthocyanidins | −0.60 | vaccinium myrtillus |
| Roseburia | polyphenols | flavonoids | −0.60 | camellia sinensis |
| Roseburia | terpenes | saponins | −0.60 | centella asiatica |
| Roseburia | polyphenols | gingerol | −0.60 | zingiber officinale |
| Roseburia | terpenes | withanolides | −0.59 | withania somnifera |
| Ruminococcus | polyphenols | anthocyanidins | −0.58 | vaccinium myrtillus |
| Ruminococcus | terpenes | saponins | −0.58 | centella asiatica |
| Ruminococcus | alkaloids | piperine | −0.58 | piper nigrum |
| Ruminococcus | alkaloids | theobromine | −0.58 | theobroma cacao |
| Ruminococcus | glycosides | saponins | −0.58 | Momordica Charantia |
| Ruminococcus | polyphenols | flavonoids | −0.57 | camellia sinensis |
| Ruminococcus | terpenes | α-pinene | −0.57 | salvia officinalis L |
| Ruminococcus | terpenes | withanolides | −0.57 | withania somnifera |
| Ruminococcus | polyphenols | flavonoids | −0.57 | ziziphus jujuba |
| Ruminococcus | polysaccharides | inulin | −0.57 | adansonia |
| Ruminococcus | polyphenols | flavonoids | −0.56 | citrus paradisi |
| Ruminococcus | curcuminoids | diarylheptanoids | −0.56 | curcuma longa |
| Slackia | glycosides | ginsenosides | −0.56 | panax ginseng |
| Slackia | polyphenols | flavones | −0.56 | gingko biloba |
| Slackia | terpenes | saponins | −0.55 | centella asiatica |
| Slackia | terpenes | saponins | −0.55 | centella asiatica |
| Sutterella | polyphenols | anthocyanidins | −0.55 | vaccinium myrtillus |
| Sutterella | curcuminoids | diarylheptanoids | −0.55 | tumeric rhizome |
| Sutterella | terpenes | saponins | −0.55 | centella asiatica |
| Sutterella | glycosides | flavonoid glycosides | −0.55 | fagopyrum esculentum |
| Sutterella | polysaccharides | beta glucan | −0.54 | hericium erinaceus |
| Sutterella | polyphenols | flavonoids | −0.54 | citrus paradisi |
| Sutterella | polyphenols | flavones | −0.53 | gingko biloba |
| Veillonella | glycosides | triterpenoid saponins | −0.53 | bacopa monnieri |
| Veillonella | terpenes | withanolides | −0.53 | withania somnifera |
| Escherichia | polyphenols | anthocyanidins | −0.53 | vaccinium myrtillus |
| Escherichia | polyphenols | flavonoids | −0.53 | gingko biloba |
| Escherichia | polyphenols | anthocyanidins | −0.53 | vaccinium myrtillus |
| Escherichia | polyphenols | phenolic acids | −0.52 | citrus sinensis |
| Escherichia | glycosides | ginsenosides | −0.52 | panax ginseng |
| Escherichia | polyphenols | flavonoids | −0.51 | ziziphus jujuba |
| Streptococcus | polyphenols | gingerol | −0.51 | zingiber officinale |
| Streptococcus | alkaloids | theobromine | −0.51 | theobroma cacao |
| Streptococcus | terpenes | boswellic acids | −0.51 | boswellia serrata |
| Streptococcus | glycosides | flavonoid glycosides | −0.50 | fagopyrum esculentum |
| Enterobacter | polyphenols | phenolic acids | −0.50 | citrus sinensis |

End of Table 5—SMs with a correlation factor of below −0.5 for more than one Bacteria. The table further indicates suitable Extract Sources.

It has been shown that SMs from different sources, such as different plant extract sources may be used to decrease specific bacteria abundancies. Some secondary metabolites, from different extract sources may be more potent than others for specific bacteria and thus provide flexibility in selection and dose, dependent on which effect is desired relative to other secondary metabolites.

The following table illustrates where correlation was particularly pronounced, i.e. a correlation factor of −0.95 and below to specific bacteria abundancies for flavonoids, beta glucan, ginsenosides, flavones, theobromine, saponins, anthocyanidins, diarylheptanoids, gingerol, boswellic acids, flavonoid glycosides and phenolic acids, all from one of the following class A categories of SMs: polyphenols, polysaccharides, terpenes, alkaloids, glycosides, terpenes and curcuminoids.

TABLE 6
SMs with a correlation factor of below −0.95 for more than one
Bacteria. The table further indicates suitable Extract Sources.

| | Secondary | Secondary | | |
|---|---|---|---|---|
| Bacteria | Metabolite | Metabolite | Correlation | |
| [genus] | Class A | Class B | Factor | Extract Source |
| Akkermansia | polyphenols | flavonoids | −1.00 | camellia sinensis |
| Bacteroides | polysaccharides | beta glucan | −1.00 | hordeum vulgare |
| Bacteroides | glycosides | ginsenosides | −1.00 | panax ginseng |
| Bacteroides | polyphenols | flavones | −1.00 | gingko biloba |
| Bacteroides | alkaloids | theobromine | −1.00 | theobroma cacao |
| Bacteroides | terpenes | saponins | −1.00 | centella asiatica |
| Bacteroides | polyphenols | anthocyanidins | −1.00 | vitis vinifera |
| Bacteroides | curcuminoids | diarylheptanoids | −1.00 | curcuma longa |
| Bacteroides | polyphenols | gingerol | −1.00 | zingiber officinale |
| Bacteroides | terpenes | boswellic acids | −1.00 | boswellia serrata |
| Bacteroides | polysaccharides | beta glucan | −1.00 | hericium erinaceus |
| Bacteroides | polyphenols | flavonoids | −1.00 | citrus paradisi |
| Bacteroides | glycosides | flavonoid glycosides | −1.00 | fagopyrum esculentum |
| Bacteroides | polyphenols | flavonoids | −1.00 | camellia sinensis |
| Bacteroides | curcuminoids | diarylheptanoids | −1.00 | tumeric rhizome |
| Bifidobacterium | polysaccharides | beta glucan | −1.00 | hordeum vulgare |
| Bifidobacterium | terpenes | boswellic acids | −1.00 | boswellia serrata |
| Bifidobacterium | polyphenols | flavonoids | −1.00 | ziziphus jujuba |
| Bifidobacterium | polyphenols | phenolic acids | −0.99 | citrus sinensis |
| Bifidobacterium | terpenes | saponins | −0.98 | centella asiatica |
| Bifidobacterium | polyphenols | anthocyanidins | −0.98 | vitis vinifera |
| Bifidobacterium | polyphenols | flavonoids | −0.97 | camellia sinensis |
| Bifidobacterium | glycosides | ginsenosides | −0.97 | panax ginseng |
| Bifidobacterium | curcuminoids | diarylheptanoids | −0.97 | tumeric rhizome |
| Bifidobacterium | curcuminoids | diarylheptanoids | −0.97 | curcuma longa |
| Blautia | polysaccharides | beta glucan | −0.96 | hordeum vulgare |
| Blautia | alkaloids | theobromine | −0.96 | theobroma cacao |
| Blautia | polyphenols | flavonoids | −0.96 | citrus paradisi |

End of Table 6—Ms with a correlation factor of below −0.95 for more than one Bacteria. The table further indicates suitable Extract Sources.

In the next table we illustrate which SMs may be selected to decrease several bacteria genus abundancies at the same time.

TABLE 7
Secondary metabolites having a negative correlation
factor of at least below −0.5 for decrease in
more than one Bacteria. (Table splits across pages)

| Bacteria [genus] | SM Class B |
|---|---|
| Akkermansia-Bacteroides-Bifidobacterium | flavonoids |
| | boswellic-acids |
| | flavones |
| | saponins |
| | anthocyanidins |
| Bacteroides-Bifidobacterium | beta-glucan |
| | ginsenosides |
| | curcuminoids |
| Bacteroides-Bifidobacterium | piperine |
| | theobromine |
| | flavanones |
| Clostridium-Coprococcus-Escherichia | anthocyanidins |
| | flavonoids |
| Clostridium-Coprococcus | α-pinene |
| | withanolides |
| | saponins |
| | flavonoid-glycoside |
| Clostridium-Escherichia | gingerol |
| | flavones |
| | ginsenosides |
| | boswellic-acids |
| Coprococcus-Escherichia | beta-glucan |
| Eubacterium-Faecalibacterium-Lactobacillus | flavonoids |
| | saponins |
| | ginsenosides |
| | inulin |
| | pectin |
| | anthocyanidins |
| Eubacterium-Faecalibacterium | α-pinene |
| | withanolides |
| | rosavins |
| | beta-glucan |
| Faecalibacterium-Lactobacillus | flavanones |
| | theobromine |
| | curcuminoids |
| Roseburia-Ruminococcus-Streptococcus | anthocyanidins |
| | flavonoids |
| | saponins |
| | withanolides |
| | flavanones |
| | piperine |
| | boswellic-acids |
| | theobromine |
| Roseburia-Ruminococcus | beta-glucan |
| | curcuminoids |
| Roseburia-Streptococcus | gingerol |
| | ginsenosides |
| | flavones |
| Ruminococcus-Streptococcus | inulin |
| | pectin |
| | flavonoid-glycoside |

End of Table 7—Secondary metabolites having a negative correlation factor of at least below −0.5 for decrease in more than one Bacteria.

### Example 3—Selected Compositions and the Log 2 Fold Increase on Selected Bacteria

The compositions specified in table A were tested and analysed in accordance with the described in vitro method in example 1.

Data was compiled and normalised to Log 2 fold change according to example 2.

The below shows effects of selected compositions according to this invention on bacteria abundance in Log 2 fold change relative to the control.

The compositions comprise secondary metabolites according to table A, however, may comprise other secondary metabolites than listed in the tables of the description.

All compositions comprise at least a total of about 10% (w/w) of SMs from at least two extract sources. See Table A and the description for details.

TABLE 8
Compositions comprising at least 9% (w/w) secondary metabolites
from at least two sources and their positive effect on
Bacteria abundancies. (Table splits across pages)

| Composition | Bacteria | Effect on Bacteria | Log2 fold |
|---|---|---|---|
| [ID] | [genus] | Abundance | change |
| FM01 | Bacteroides | increase | 2.22 |
| FM02 | Bacteroides | increase | 3.70 |
| FM03 | Bacteroides | increase | 1.10 |
| FM30 | Bacteroides | increase | 1.22 |
| FM31 | Bacteroides | increase | 1.67 |
| FM32 | Bacteroides | increase | 1.78 |
| MRSM1 | Bacteroides | increase | 2.07 |
| NPM1 | Bacteroides | increase | 0.94 |
| NPM2 | Bacteroides | increase | 0.47 |
| NPM3 | Bacteroides | increase | 0.54 |
| RM1 | Bacteroides | increase | 0.50 |
| RM10 | Bacteroides | increase | 1.05 |
| RM11 | Bacteroides | increase | 1.72 |
| RM12 | Bacteroides | increase | 3.21 |
| CM01 | Bifidobacteria | increase | 5.06 |
| CM02 | Bifidobacteria | increase | 7.68 |
| FM01 | Bifidobacteria | increase | 2.57 |
| FM02 | Bifidobacteria | increase | 4.07 |
| FM03 | Bifidobacteria | increase | 2.97 |
| FM30 | Bifidobacteria | increase | 2.79 |
| FM31 | Bifidobacteria | increase | 4.06 |
| FM32 | Bifidobacteria | increase | 3.92 |
| MEM3 | Bifidobacteria | increase | 1.57 |
| MEM7 | Bifidobacteria | increase | 1.68 |
| MM11 | Bifidobacteria | increase | 6.62 |
| MRSM1 | Bifidobacteria | increase | 1.96 |
| NPM3 | Bifidobacteria | increase | 0.81 |
| RM1 | Bifidobacteria | increase | 4.17 |
| RM10 | Bifidobacteria | increase | 5.45 |
| RM11 | Bifidobacteria | increase | 5.01 |
| RM12 | Bifidobacteria | increase | 3.24 |
| TSM120 | Bifidobacteria | increase | 1.75 |
| TSM20 | Bifidobacteria | increase | 2.17 |
| FM01 | Clostridium | increase | 1.85 |
| FM03 | Clostridium | increase | 1.65 |
| FM30 | Clostridium | increase | 2.75 |
| FM32 | Clostridium | increase | 1.69 |
| RM1 | Clostridium | increase | 1.37 |
| RM10 | Clostridium | increase | 2.25 |
| RM11 | Clostridium | increase | 0.67 |
| RM12 | Clostridium | increase | 2.80 |
| FM32 | Coprococcus | increase | 2.06 |
| NPM1 | Coprococcus | increase | 0.40 |
| RM1 | Coprococcus | increase | 1.24 |
| RM10 | Coprococcus | increase | 1.24 |
| RM12 | Coprococcus | increase | 1.90 |
| FO61 | Escherichia | increase | 1.72 |
| FO62 | Escherichia | increase | 1.93 |
| FO63 | Escherichia | increase | 0.40 |
| FO64 | Escherichia | increase | 0.93 |
| FO65 | Escherichia | increase | 0.86 |
| FO68 | Escherichia | increase | 0.82 |
| FO69 | Escherichia | increase | 0.32 |
| FO70 | Escherichia | increase | 1.29 |
| FO71 | Escherichia | increase | 0.52 |
| FO72 | Escherichia | increase | 0.97 |
| FO73 | Escherichia | increase | 0.80 |
| FO74 | Escherichia | increase | 1.46 |
| FO77 | Escherichia | increase | 1.15 |
| FO78 | Escherichia | increase | 0.40 |
| FO80 | Escherichia | increase | 0.84 |
| RM10 | Escherichia | increase | 0.55 |
| MEM3 | Faecalibacterium | increase | 0.97 |
| MEM7 | Faecalibacterium | increase | 2.37 |
| NPM1 | Faecalibacterium | increase | 1.86 |
| NPM2 | Faecalibacterium | increase | 0.41 |
| CM01 | Lactobacillus | increase | 10.92 |
| CM02 | Lactobacillus | increase | 8.93 |
| FM01 | Lactobacillus | increase | 3.69 |
| MEM3 | Lactobacillus | increase | 3.10 |
| MEM7 | Lactobacillus | increase | 1.90 |
| MM11 | Lactobacillus | increase | 3.28 |
| MRSM1 | Lactobacillus | increase | 7.01 |
| NPM1 | Lactobacillus | increase | 10.97 |
| NPM2 | Lactobacillus | increase | 12.55 |
| NPM3 | Lactobacillus | increase | 12.69 |
| FM01 | Roseburia | increase | 0.91 |
| FM02 | Roseburia | increase | 0.65 |
| FM32 | Roseburia | increase | 0.79 |
| MEM3 | Roseburia | increase | 2.77 |
| MEM7 | Roseburia | increase | 4.58 |
| MM11 | Roseburia | increase | 0.91 |
| MRSM1 | Roseburia | increase | 0.42 |
| NPM1 | Roseburia | increase | 1.22 |
| FO61 | Streptococcus | increase | 1.68 |
| FO62 | Streptococcus | increase | 1.69 |
| FO64 | Streptococcus | increase | 1.45 |
| FO66 | Streptococcus | increase | 1.15 |
| FO68 | Streptococcus | increase | 0.61 |
| FO70 | Streptococcus | increase | 0.64 |
| FO72 | Streptococcus | increase | 0.92 |
| FO73 | Streptococcus | increase | 0.53 |
| FO74 | Streptococcus | increase | 1.06 |
| FO79 | Streptococcus | increase | 0.71 |
| FO82 | Streptococcus | increase | 1.12 |
| FO83 | Streptococcus | increase | 0.95 |
| PPM12 | Streptococcus | increase | 1.45 |
| PPM120 | Streptococcus | increase | 0.92 |

End of Table 8—Compositions comprising at least 9% (w/w) secondary metabolites from at least two sources and their positive effect on Bacteria abundancies.

### Example 4a—Comparing Compositions Comprising Different Amounts of Polyphenols and Glycosides

The compositions specified in table A were tested and analysed in accordance with the described in vitro method in example 1.

Data was compiled and normalised to Log 2 fold change according to example 2.

The below shows effects of selected compositions according to their effect on bacteria abundance in Log 2 fold change relative to the control.

From the data shown in the below table it is shown that the effect of at least two SMs vs. only one SM on the bacteria abundance is improved.

Further, a similar improved effect was also detected for combinations, such as e.g., polyphenol and glycosides or polyphenols, alkaloids and/or glycosides and terpens.

Particular emphasis is made on the results shown for compositions comprising at least two SMs, wherein said SMs constitute at least 8% (w/w), preferably 10% (w/w).

TABLE 9
Comparing compositions comprising different amounts of
polyphenols and glycosides on clostridium abundancy.

| | | | | | | | | | Log 2 fold |
|---|---|---|---|---|---|---|---|---|---|
| Formula | SMs class A//SMs class A % (w/w) in composition | | | | | | | | Change |
| F061 | polyphenols | 0.0 | glycosides | 13.5 | alkaloids | 23.8 | vitamins | 0.0 | 3.73 |
| F045 | | 15.0 | | 0.0 | alkaloids/ | 0.0 | | 0.0 | 1.29 |
| F065 | | 0.0 | | 27.0 | terpenes | 0.0 | | 0.0 | 1.68 |
| F055 | | 18.0 | | 5.0 | | 0.0 | | 0.0 | 2.12 |
| F054 | | 12.5 | | 5.0 | | 0.0 | | 0.0 | 2.25 |
| F052 | | 15.3 | | 2.5 | | 0.0 | | 0.0 | 2.39 |
| F053 | | 20.1 | | 3.3 | | 0.0 | | 0.0 | 2.66 |
| F059 | | 12.5 | | 25.0 | | 0.0 | | 0.0 | 3.13 |
| F058 | | 47.5 | | 25.0 | | 0.0 | | 0.0 | 3.63 |
| F057 | | 39.6 | | 16.5 | | 0.0 | | 0.0 | 4.29 |
| F062 | | 32.3 | | 17.8 | | 0.0 | | 0.0 | 4.43 |
| F060 | | 30.0 | | 12.5 | | 0.0 | | 0.0 | 4.44 |
| F056 | | 30.0 | | 12.5 | | 0.0 | | 0.0 | 5.14 |
| FO66 | | 49.0 | | 25.0 | | 0.0 | | 0.0 | 5.74 |
| FM30 | | 7.8 | | 27.0 | terpenes | 5.3 | | 4.3 | 2.75 |

End of Table 9—Comparing compositions comprising different amounts of polyphenols and glycosides.

The below table shows how different bacteria are effected in different ways by SMs combinations, either enhancing, neutralizing or reducing the effects.

TABLE 9a
Effect of glycoside, phenols and combination on a series of other bacteria abundancies.

| Compo- | Log 2 fold chage of Bacteria abundancy | | | | | | | |
|---|---|---|---|---|---|---|---|---|
| sition | | Bifido- | | | | | | |
| [ID] | Bacteroides | bacterium | Blautia | Coprococcus | Dorea | Ruminococcus | Escherichia | Streptococcus |
| FM30 | 1.22 | 2.79 | −0.73 | −2.47 | −2.77 | −4.35 | 1.04 | 0.81 |
| FO61 | −3.90 | 2.79 | −1.09 | −1.81 | −1.24 | −3.52 | 1.72 | 1.68 |
| FO56 | −0.59 | 1.74 | −2.44 | −2.45 | −4.52 | −5.61 | 0.58 | 3.89 |
| FO58 | −4.37 | 2.88 | −1.17 | −0.29 | −2.60 | −3.79 | 1.38 | 1.14 |
| FO61 | −3.90 | 2.79 | −1.09 | −1.81 | −1.24 | −3.52 | 1.72 | 1.68 |
| FO65 | −2.46 | 2.63 | −2.34 | −0.51 | 1.30 | −4.88 | 0.86 | 0.18 |
| FO45 | −2.94 | 4.05 | −2.06 | 1.37 | 1.15 | −3.37 | −3.77 | −0.72 |

TABLE 10
Direct comparison of effect on Coprococcus abundancy
of compositions comprising 13% (w/w) Polyphenols
and 5% (w/w) and 25% (w/w) glycosides respectively.

| Composition [ID] | Log2 fold change Clostridium | Log2 fold chang |
|---|---|---|
| FO54 | 2.25 | −2.04 |
| FO59 | 3.13 | 0.49 |

TABLE 11
Direct comparison of effect on Coprococcus & Clostridium
abundancies of compositions comprising 13-22% Polyphenols
and 0%, 3%, 3%, 5%,5% and 25% glycosides respectively.

| | Log2 change | Log2 fold change |
|---|---|---|
| Composition [ID] | Clostridium | Coprococcus |
| FO45 | 1.29 | 1.37 |
| FO52 | 2.39 | −2.32 |
| FO53 | 2.66 | −1.71 |
| FO54 | 2.25 | −2.04 |
| FO55 | 2.12 | −2.14 |
| FO59 | 3.13 | 0.49 |

It is evident that even small amounts of glycosides in combination with polyphenols have an increasing effect that is even further enhanced by an additional increase of glycosides.

Combinations of other SMs combinations on specific bacteria abundancies have been tested, but now shown here.

### Example 4—Selected Data Presented for the Composition Tested and Disclosed Herein

This example presents a selection of data based on the compositions tested in our laboratory. The compositions specified in table A were tested and analysed in accordance with the described in vitro method in example 1. Data was compiled and normalised to Log 2 fold change according to example 2.

### Example 4a—Comprising Compositions Com-Rising Different Amounts of Polyphenols or Glycosides and Curcuminoids, Terpenes and/or Polysaccharides

The below data shows an overview of the compositions analysed and the effect of such selected compositions on bacteria abundance in Log 2 fold change relative to the control.

TABLE 12
Composition comprising polyphenols (0-58% (w/w)), polysaccharides (0-18%
(w/w) and either comprise or do not comprise curcuminoids 0.6-48% (w/w).

| | | class | | Class | | Class | | Class |
|---|---|---|---|---|---|---|---|---|
| Compo- | | SM A % | | SM A % | | SM A % | | SM A % |
| sition | Class A | (w/w) in | Class A | (w/w) in | Class A | (w/w) in | Class A | (w/w) in |
| [ID] | SM | composition | SM | composition | SM | composition | SM | composition |
| F042 | polyphenols | 14.00 | curcuminoids | 0.00 | polysaccharides | 14.00 | other | 0.00 |
| F043 | | 18.48 | | 0.00 | | 0.00 | | 0.00 |
| F044 | | 15.50 | | 0.00 | | 0.00 | | 0.00 |
| F045 | | 15.00 | | 0.00 | | 0.00 | | 0.00 |
| F046 | | 25.50 | | 0.00 | | 0.00 | | 0.00 |
| F047 | | 6.25 | | 23.75 | | 13.25 | | 0.00 |
| F048 | | 8.25 | | 31.35 | | 8.25 | | 0.00 |
| F049 | | 0.00 | | 47.50 | | 12.50 | | 0.00 |
| F050 | | 12.50 | | 0.00 | | 12.50 | | 0.00 |
| F051 | | 12.50 | | 47.50 | | 0.00 | | 0.00 |
| F070 | | 32.34 | | 0.00 | | 17.49 | | 0.00 |
| F072 | | 24.75 | | 0.00 | | 17.50 | alka- | 23.75 |
| | | | | | | | loids | |
| MEM7 | | 7.11 | | 0.67 | | 0.00 | other | 3.50 |
| PPM10 | | 32.75 | | | | 14.00 | | 0.00 |
| PPM12 | | 57.25 | | 23.75 | | 0.00 | | 0.00 |
| PPM120 | | 42.94 | | 17.81 | | 7.00 | | 0.00 |
| PPM20 | | 24.50 | | 23.75 | | 14.00 | | 0.00 |
| FM02 | | 12.13 | glyco- | 1.38 | | 3.29 | | 4.50 |
| | | | sides | | | | | |
| NPM2 | | 24.00 | glyco- | 1.00 | | 0.00 | other | 0.00 |
| | | | sides | | | | | |

Looking at selected bacteria abundancies for two compositions only comprising polyphenols (FO44 and FO45) it can be concluded that bifidobacterim and clostridium abundancies are increased, whereas blautia and rumibococcus are decreased by polyphenols if they are administered on their own.

Adding curcuminoids (F051) to the mix reveals an effect on ruminococcus abundance, which is further decreased compared to the effect of polyphenoles alone. Curcuminoids also affect clostridium abundance which is further increased and faecalibacterium abundancy in which the decrease of bacteria abundancy is reduced.

TABLE 13
Effect of a series of bactera abundancies of
polyphenoles in combination with curcuminoids

| Compo- | | | | | | |
|---|---|---|---|---|---|---|
| sitions | | Bifido- | | | | Faecali- |
| [ID] | Bacteroides | bacterium | Blautia | Ruminococcus | Clostridium | bacterium |
| FO51 | −2.68 | 3.69 | −2.98 | −4.31 | 2.42 | −1.39 |
| FO44 | −2.46 | 3.57 | −3.81 | −2.07 | 0.88 | −2.10 |
| FO45 | −2.94 | 4.05 | −2.06 | −3.37 | 1.29 | −2.21 |

TABLE 14
Effect on bacteriodes abundancy by comprising
not comprising curcuminoids, but polyphenols.

| Composition [ID] | Log2 change Bacteroides |
|---|---|
| FM01 | 2.22 |
| FM02 | 3.70 |
| FO42 | −1.56 |
| FO50 | −2.32 |
| FO70 | −1.36 |
| FO70 | 2.65 |
| FO72 | −5.23 |
| FO72 | 0.14 |

From the above data it is evident that a combination of high polyphenol and polysaccharides have a decreasing effect on bacteroides, whereas modest concentrations of polyphenols in combination with low or high polysaccharides increase bacteriocides abundancy.

Looking at compositions that are very similar in terms of high polyphenol and polysaccharide content but differ in that; PPM20 has about 24% (w/w) curcuminoids added, whereas F075 does not comprise curcuminoids, it is clear that curcuminoids have an effect on the composition's bacteria abundance effect. Highlighting a few of these effects; The reduction of faecalibacterium and bacteriodes is further enhanced, whereas the bifidobcterium and clostrium increasing effects are enhanced by the addition of curcuminoids. A decrease is turned around to an increasing effect in ruminococcus and coprococcis.

The bacteria abundancy change is provided in Log 2 according to example 2.

TABLE 15
Effect on a series of bacteria [genus] abundancies of compositions
comprising curcuminoids, polyphenols and polysaccharides.

| Compo- | | | | | | | | |
|---|---|---|---|---|---|---|---|---|
| sitions | | Bifido- | | | | Faecali- | | |
| [ID] | Bacteroides | bacterium | Blautia | Clostridium | Coprococcus | bacterium | Ruminococcus | Roseburia |
| PPM20 | −2.56 | 2.43 | −1.54 | 2.83 | −0.57 | −1.11 | −3.84 | −1.04 |
| FO75 | −2.81 | 3.53 | −0.71 | 4.26 | 0.47 | −3.30 | 3.54 | −0.87 |

### Example 5—Examples of Selected Compositions Comprising More than 3 SMs

This example presents a selection of complex compositions comprising at least two secondary metabolites. All compositions are provided in a capsule and the compositions weigh about 400 mg each without capsule material. All compositions are provided in a capsule and the compositions weigh about 400 mg each without capsule material. The daily administration of said compositions may be one capsule per day or two capsules per day. In some instances, the compositions may be combined.

TABLE 16
A composition comprising polyphenols, glycosides, terpenes and vitamins as shown
in the below table by dry weight of the extract (Table splits across pages)

| Extract % | | | | Extract in | Equivalent to |
|---|---|---|---|---|---|
| (w/w) in | | | | Extract | dry natural |
| Extract | Extract | | | mixture | material |
| mixture | Source | SM Class A | SM Class B | (mg) | (mg) |
| 22 | rhodiola rosea | glycosides | rosavins | 90 | 990 |
| 24 | withania somnifera | terpenes | withanolides | 95 | 760 |
| 7 | hypericum perforatum | glycosides | flavonoid | 35 | 105 |
| | | | glycosides | | |
| 10 | melissa officinalis | polyphenols | rosmarinic acid, | 40 | 25.5 |
| | | | hydroxycinnamic | | |
| | | | acid | | |
| 1 | rice extract | filling agent | filling agent | 2.5 | not |
| | | (not SM) | (not SM) | | calculated |
| 9 | valeriana officinalis | polyphenols | apigenin flavone | 30 | 225 |
| | | | glycoside | | |
| 7 | passiflora incarnata | polyphenols | vexitin | 45 | 220 |
| 19 | prunus cerasus | polyphenols | anthocyanin | 70 | 1850 |

In one embodiment, the composition comprises at least a total of about 24-27% (w/w) SMs, about 23% (w/w) polyphenols, about 1% (w/w) glycosides, and about 0.5% (w/w) terpenes by dry weight of the extract. In addition, the composition comprises about 1% (w/w) filling agent (not SM). This effect of combining polyphenols, glycosides and polysaccharides is reviewed in example 1-4.

TABLE 17
A composition comprising polyphenols, glycosides, polysaccharides, terpenes and vitamins
as shown in the below table by dry weight of the extract. (Table splits across pages)

| Extract % | | | | Extract in | Equivalent to |
|---|---|---|---|---|---|
| (w/w) in | | | | Extract | dry natural |
| Extract | | | | mixture | material |
| mixture | Extract Source | SM Class A | SM Class B | (mg) | (mg) |
| 20 | rhodiola rosea | glycosides | rosavins | 80 | 720 |
| 6 | vitis vinifera | polyphenols | anthocyanidins | 25 | 625 |
| 5 | ziziphus jujuba | polyphenols | flavonoids | 20 | 200 |
| 19 | withania somnifera | terpenes | withanolides | 75 | 600 |
| 2 | riboflavin | vitamins | vitamin B2 | 7.5 | not calculated |
| 2 | cholecalciferol | vitamins | vitamin D3 | 19 | not calculated |
| 20 | avena sativa L | poly-saccharides | beta glucan | 80 | 720 |
| 5 | salvia officinalis L | terpenes | α-pinene | 20 | 150 |
| 10 | centella asiatica | terpenes | saponins | 40 | 960 |
| 10 | camellia sinensis | polyphenols | flavonoids | 40 | 720 |
| 1 | rice extract | filling agent | filling agent | 20 | not calculated |
| | | (not SM) | (not SM) | | |

In one embodiment, the composition comprises at least a total of about 27-30% (w/w), preferably about 28% SMs, about 0.5-1% (w/w) glycosides, preferably about 0.6% (w/w), about 16% (w/w) polyphenols, about 6% (w/w) polysaccharides, about 1.5% (w/w) terpenes about 4% (w/w) vitamins by dry weight of the extract. In addition, the composition comprises about 1% (w/w) filling agent (not SM). This effect of combining polyphenols, glycosides and polysaccharides is reviewed in example 1-4.

TABLE 18
A composition comprising polyphenols, glycosides, polysaccharides,
and alkaloids as shown in the below table by dry weight
of the extract. (Table splits across pages)

| Extract % | | | | Extract in | Equivalent to |
|---|---|---|---|---|---|
| (w/w) in | | | | Extract | dry natural |
| Extract | Extract | | | mixture | material |
| mixture | Source | SM Class A | SM Class B | (mg) | (mg) |
| 19% | Trigonella | glycosides | saponins | 75 | 938 |
| | foenum-graecum | | | | |
| 6% | camellia sinensis | polyphenols | flavonoids | 25 | 450 |
| less | piper nigrum | alkaloids | piperine | 1 | 45 |
| than 1% | | | | | |
| 30% | citrus limon | polysaccharides | pectin | 120 | 2400 |
| 10% | citrus paradisi | polyphenols | flavonoids | 40 | 800 |
| 19% | Mormodica | glycosides | triterpene | 75 | 300 |
| | charantia | | glycosides | | |
| 1% | rice extract | filling agent | filling agent | 2 | not |
| | | (not SM) | (not SM) | | calculated |
| 15% | theobroma cacao | alkaloids | theobromine | 60 | 285 |

In one embodiment, the composition comprises at least a total of about 52-57% (w/w), preferably about 54% (w/w) SMs, about 16% (w/w) polyphenols, about 21% (w/w) polysaccharides and about 5% (w/w) alkaloids and 12% (w/w) glycosides by dry weight of the extract. The composition further comprises about 1% (w/w) of a filling agent (not SM). The effect of combining polyphenols and polysaccharides, as well as terpenes is reviewed in example 1-4.

TABLE l
19- A composition comprising polyphenols, glycosides, terpenes, polysaccharides,
and vitamins as shown in the below table by dry weight of the extract.

| Extract % | | | | Extract in | Equivalent to |
|---|---|---|---|---|---|
| (w/w) in | | | | Extract | dry natural |
| Extract | | | | mixture | material |
| mixture | Extract Source | SM Class A | SM Class B | (mg/μg) | (mg) |
| 20 | avena sativa L | polysaccharides | beta glucan | 80(mg) | 720 |
| 20 | bacopa | glycosides | triterpenoid | 80(mg) | 520 |
| | monnieri | | saponins | | |
| 6 | boswellia | terpenes | boswellic acids | 25(mg) | 162.5 |
| | serrata | | | | |
| less | cyanocobalamin | vitamins | vitamin B12 | 200(μg) | not |
| than 1 | | | | | calculated |
| 20 | gingko biloba | polyphenols | flavones | 80(mg) | 1880 |
| 5 | humulus hupulus | polyphenols | flavonoids | 20(mg) | 250 |
| 1 | rice extract | filling agent | filling agent | 1(mg) | not |
| | | (not SM) | (not SM) | | calculated |
| 2 | pyridoxine | vitamins | vitamin B6 | 8(mg) | not |
| | | | | | calculated |
| 10 | panax ginseng | glycosides | ginsenosides | 62.5(mg) | 312.5 |
| 10 | vaccinium | polyphenols | anthocyanidins | 40(mg) | 1800 |
| | myrtillus | | | | |

In one embodiment, the composition comprises about 20-23% (w/w), preferably about 22% (w/w) SMs, about 8% (w/w) polyphenols, about 2.5% (w/w) glycosides, about 6% (w/w) polysaccharides, about 2% (w/w) vitamins and about 4% (w/w) terpenes by dry weight of the extract. In addition, the composition comprises about 1% (w/w) filling agent (not SM). The effect of this combination is reviewed in example 1-4.

TABLE 20
A composition comprising polyphenols, curcuminoids, polysaccharides and
vitamins as shown in the below table by dry weight of the extract.

| Extract % | | | | Extract in | Equivalent to |
|---|---|---|---|---|---|
| (w/w) in | | | | Extract | dry natural |
| Extract | Extract | | | mixture | material |
| mixture | Source | SM Class A | SM Class B | (mg) | (mg) |
| 9 | allium sativum | polyphenols | anthocyanidins | 35 | 350 |
| 15 | chicorium | polysaccharides | inulin | 60 | 360 |
| | intybus | | | | |
| 8 | citrus sinensis | polyphenols | flavanoid | 32.5 | 390 |
| 16 | malpighia | vitamins | vitamin C | 65 | 1365 |
| | glabra | | | | |
| 1 | rice extract | filling agent | filling agent | 2 | not |
| | | (not SM) | (not SM) | | calculated |
| 24 | rosa canina | vitamins | vitamin C | 95 | 380 |
| 10 | tumeric | curcuminoids | diarylheptanoids | 40 | 1200 |
| | rhizome | | | | |
| 9 | vaccinium | polyphenols | anthocyanidins | 35 | 675 |
| | myrtillus | | | | |
| 8 | zingiber | polyphenols | gingerol | 33 | 488 |
| | officinale | | | | |

In one embodiment, the composition comprises at least a total of at least 26-30% (w/w) preferably about 28% (w/w) SMs, about 8% (w/w) polyphenols, about 3% (w/w) curcuminoids, about 2% (w/w) polysaccharides and about 15% (w/w) vitamins. The composition further comprises about 1% (w/w) filling agent (not SM). The effects of this combination is reviewed in example 1-4.

1. A composition comprising an extract, said extract comprising at least two plant secondary metabolites, wherein said at least two plant secondary metabolites are encapsulated by an acid resistant coating and wherein each plant secondary metabolites constitutes less than about 50% (w/w) by dry weight of the extract.

2. The composition according to claim 1, wherein said extract is a mixture of several extracts and wherein said at least two plant secondary metabolites constitute at least 10% (w/w) by dry weight of the extract.

3. The composition according to claim 1, wherein said coating dissolves at a pH-value of above pH 4.

4. The composition according to claim 1, comprising one or more additional pharmaceutically and/or nutritionally acceptable excipients and/or minerals.

5. The composition according to claim 1, wherein said at least two plant secondary metabolites are from different extract sources.

6. The composition according to claim 1, wherein each of said at least two plant secondary metabolites may be contained in any proportion in the extract, as long as the total amount of the plant secondary metabolites constitutes at least 10% (w/w) by dry weight of the extract.

7. The composition according to claim 1, wherein said at least two plant secondary metabolites are independently selected from one or more of the following extract sources:
a) plant extracts, including plant extracts wherein one or more plant secondary metabolites have been enriched or purified, compared to the original dried plant source
b) yeast extracts, wherein the plant secondary metabolites are produced in genetically modified microorganisms engineered to produce plant secondary metabolites
c) fungi extract sources, including fungi extract sources, wherein one or more plant secondary metabolites have been enriched compared to the original dried fungi source

8. The composition according to claim 7, wherein said at least two plant secondary metabolites are from different plant extracts.

9. The composition according to claim 1, wherein said at least two plant secondary metabolites are independently selected from the group consisting of alkaloids, amino acids, curcuminoids, glycosides, polyphenols, polysaccharides, terpenes, and vitamins, optionally at least one plant secondary metabolite is not a polysaccharide.

10. The composition according to claim 1, wherein said extract comprises said at least two plant secondary metabolites independently selected from one or more of the combinations a)-u): a) polyphenols and one or more additional plant secondary metabolite, independently selected from the list consisting of alkaloids, amino acids, curcuminoids, glycosides, polysaccharides, terpenes, and vitamins; b) glycosides and one or more additional plant secondary metabolite selected from the list consisting of alkaloids, amino acids, curcuminoids, polyphenols, polysaccharides, terpenes, and vitamins; d) polyphenols and glycosides; e) polyphenols and polysaccharides; f) polyphenols and vitamins; g) polyphenols and terpenes; h) polyphenols and alkaloids; i) polyphenols and curcuminoids; j) glycosides and vitamins; k) glycosides and terpenes; l) glycosides and alkaloids; m) terpenes and vitamins; n) curcuminoids and polysaccharides; o) curcuminoids and vitamins; p) polysaccharides and alkaloids; q) polysaccharides and alkaloids; r) polyphenols, glycosides, terpenes and vitamins; s) polyphenols, glycosides, polysaccharides, terpenes and vitamins; t) polyphenols, glycosides, polysaccharides and alkaloids; u) polyphenols, curcuminoids, polysaccharides and vitamins.

11. The composition according to claim 9, wherein said at least two plant secondary metabolites of said group consisting of alkaloids, amino acids, curcuminoids, glycosides, polyphenols, polysaccharides, terpenes and vitamins are independently selected from any one or more of the below listed groups A-I:
A. Alkaloids; selected from the group consisting of: piperine and theobromine;
B. amino acids; selected from the group consisting of: essential amino acids and non-essential amino acids and proteinogenic amino acids also;
C. curcuminoids; selected from the group consisting of: diarylheptanoids;
D. glycosides; selected from the group consisting of: flavonoid glycosides, ginsenosides, rosavins, saponins, triterpene glycosides and triterpenoid saponins;
E. polyphenols; selected from the group consisting of: anthocyanidins, anthocyanin, apigenin flavone glycoside, flavonoid, flavones, flavonoids, gingerol, phenolic acids;
F. rosmarinic acid, hydroxycinnamic acid and vexitin;
G. polysaccharides; selected from the group consisting of: beta glucan, inulin and pectin
H. terpenes; selected from the group consisting of: boswellic acids, valerenic acid, α-pinene,
I. vitamins; selected from the group consisting of: vitamin A, B, C, D, E, K.

12. The composition according to claim 1, wherein said at least two plant secondary metabolite are selected according to claim 10 and wherein said plant secondary metabolites are provided by at least two plant extracts, optionally extracted from one or more of the plants selected from the list consisting of: adansonia, Allium sativum, Avena sativa L, Bacopa monnieri, Boswellia serrata, Camellia sinensis, Centella asiatica, Chicorium intybus, cholecalciferol, Citrus limon, Citrus paradisi, Citrus sinensis, Curcuma longa, cyanocobalamin, Fagopyrum esculentum, Gingko biloba, Griffonia simplicifolia, Hericium erinaceus, Hordeum vulgare, Humulus lupulus, Hypericum perforatum, Malpighia glabra, Melissa officinalis, Momordica charantia, Mormodica charantia, Panax ginseng, Panax ginseng, Passiflora incarnata, Piper nigrum, Prunus cerasus, pyridoxine, Rhodiola rosea, riboflavin, Rosa canina, Salvia officinalis L, Salvia officinalis L, Taraxacum officinale, Theobroma cacao, Trigonella foenum-graecum, Tumeric rhizome, Vaccinium myrtillus, Valeriana officinalis, Vitis vinifera, Withania somnifera, Zingiber officinale and Ziziphus jujuba.

13. The composition according to claim 1, wherein said at least two plant secondary metabolites or said composition are encapsulated in an acid resistant coating and wherein said encapsulation is a microencapsulation or a nanoencapsulation or wherein said extract comprising several extracts is provided in a capsule resistant to dissolution in the stomach acid.

14. The composition according to claim 1, comprising extracts and/or pharmaceutically and/or nutritionally acceptable excipients selected from any one of the following compositions a)-e):
a) about 24-27% (w/w) plant secondary metabolites, including vitamins and said composition further comprises about 1.5% (w/w) filing agent,
wherein said plant extract comprises 26-30% (w/w), preferably about 28% (w/w) or about 90 mg Rhodiola rosea root extract, which is equivalent to about 990 mg dry unmodified Rhodiola rosea root; 22-26% (w/w), preferably about 24% (w/w) or about 95 mg Withania somnifera root extract, which is equivalent to about 760 mg dry unmodified Withania somnifera root;
6-9% (w/w), preferably about 7-8% (w/w) or about 35 mg Hypericum perforatum plant (aerial part) extract, which is equivalent to about 105 mg dry unmodified Hypericum perforatum plant (aerial part); 8-12% (w/w), preferably about 10% (w/w) or about 40 mg Melissa officinalis leaf extract, which is equivalent to about 265 mg dry unmodified Melissa officinalis leaf, 7-11% (w/w), preferably about 9% (w/w) or about 30 mg Valeriana officinalis root extract, which is equivalent to about 225 mg dry unmodified Valeriana officinalis root; 5-9% (w/w), preferably about 7% (w/w) or about 30 mg Passiflora incarnata flower extract, which is equivalent to about 225 mg dry unmodified Passiflora incarnata flower; and 17-21% (w/w), preferably about 19% (w/w) or about 75 mg Prunus cerasus fruit extract, which is equivalent to about 1875 mg dry unmodified Prunus cerasus fruit;
b) about 24-27% (w/w) plant secondary metabolites, including vitamins and said composition further comprises about 1% (w/w) filing agent,
wherein said plant extract comprises 18-22% (w/w), preferably about 20% (w/w) or about 80 mg Rhodiola rosea root extract, which is equivalent to about 720 mg dry unmodified Rhodiola rosea root; 4-8% (w/w), preferably about 6% (w/w) or about 25 mg Vitis vinifera seed extract, which is equivalent to about 625 mg dry unmodified Vitis vinifera seed; 3-7% (w/w), preferably about 5% (w/w) or about 20 mg Ziziphus jujuba fruit extract, which is equivalent to about 200 mg dry Ziziphus jujuba fruit; 17-21% (w/w), preferably about 19% (w/w) or about 75 mg Withania somnifera root extract, which is equivalent to about 600 mg dry unmodified Withania somnifera root; 0-4% (w/w), preferably about 2% (w/w) or about 7.5 mg riboflavin;
0-4% (w/w), preferably about 2% (w/w) or about 38 mg cholecalciferol; 18-22% (w/w), preferably about 18% (w/w) or about 80 mg Avena sativa L extract, which is equivalent to about 720 mg dry unmodified Avena sativa L; 3-8% (w/w), preferably about 5% (w/w) or about 20 mg Salvia officinalis L bran extract, which is equivalent to about 150 mg dry unmodified salvia officinalis L bran; 8-12% (w/w), preferably about 8% (w/w) or about 40 mg Centella asiatica plant extract, which is equivalent to about 960 mg dry unmodified Centella asiatica plant; and 8-12% (w/w), preferably about 10% (w/w) or about 40 mg Camellia sinensis leaf extract, which is equivalent to about 720 mg dry unmodified Camellia sinensis leaf,
c) about 24-27% (w/w) plant secondary metabolites, including vitamins and said composition further comprises about 1% (w/w) filing agent,
wherein said plant extract comprises 17-21% (w/w), preferably about 19% (w/w) or about 75 mg Trigonella foenum-graecum seed extract, which is equivalent to about 938 mg dry unmodified Trigonella foenum-graecum seed; 4-8% (w/w), preferably about 6% (w/w) or about 25 mg Camellia sinensis leaf extract, which is equivalent to about 450 mg dry unmodified Camellia sinensis leaf, 0-3% (w/w), preferably about 0.2% (w/w) or about 1 mg Piper nigrum fruit extract, which is equivalent to about 450 mg dry Piper nigrum fruit; 28-32% (w/w), preferably about 30% (w/w) or about 120 mg Citrus limon skin extract, which is equivalent to about 2400 mg dry unmodified Citrus limon skin; 8-12% (w/w), preferably about 10% (w/w) or about 40 mg Citrus paradisi fruit extract, which is equivalent to about 800 mg dry unmodified citrus fruit paradisi; 17-21% (w/w), preferably about 19% (w/w) or about 75 mg Mormodica charantia fruit extract, which is equivalent to about 300 mg dry unmodified Mormodica charantia fruit; and 13-17% (w/w), preferably about 15% (w/w) or about 60 mg Theobroma cacao bean extract, which is equivalent to about 285 mg dry unmodified Theobroma cacao bean;
d) about 24-27% (w/w) plant secondary metabolites, including vitamins and said composition further comprises about 1% (w/w) filing agent,
wherein said plant extract comprises 18-22% (w/w), preferably about 20% (w/w) or about 80 mg Avena sativa L bran extract, which is equivalent to about 720 mg dry unmodified Avena sativa L bran; 18-22% (w/w), preferably about 20% (w/w) or about 80 mg Bacopa monnieri plant extract, which is equivalent to about 520 mg dry unmodified Bacopa monnieri plant; 4-8% (w/w), preferably about 6% (w/w) or about 25 mg Boswellia serrata plant extract, which is equivalent to about 163 mg dry Boswellia serrata perforatum plant; 18-22% (w/w), preferably about 18% (w/w) or about 80 mg Gingko biloba leaf extract, which is equivalent to about 1880 mg dry unmodified Gingko biloba leaf, 8-12% (w/w), preferably about 10% (w/w) or about 40 mg Vaccinium myrtillus fruit extract, which is equivalent to about 1800 mg dry unmodified Vaccinium myrtillus fuit; 0-2% (w/w), preferably about 0.05% (w/w) or about 200 μg cyanocobalamin; 0-4% (w/w), preferably about 2% (w/w) or about 8 mg pyridoxine; and 8-12% (w/w), preferably about 10% (w/w) or about 62.5 mg Panax ginseng root extract, which is equivalent to about 313 mg dry unmodified Panax ginseng root;
e) about 24-27% (w/w) plant secondary metabolites, including vitamins and said composition further comprises about 1% (w/w) filing agent,
wherein said plant extract comprises 7-11% (w/w), preferably about 9% (w/w) or about 35 mg Allium sativum bulb extract, which is equivalent to about 350 mg dry unmodified Allium sativum bulb; 13-17% (w/w), preferably about 15% (w/w) or about 60 mg Chicorium intybus plant extract, which is equivalent to about 360 mg dry chicorium plant intybus; 6-10% (w/w), preferably about 8% (w/w) or about 33 mg Citrus sinensis skin extract, which is equivalent to about 390 mg dry Citrus sinensis skin; 14-18% (w/w), preferably about 16% (w/w) or about 65 mg Malpighia glabra fruit extract, which is equivalent to about 1365 mg dry unmodified Malpighia glabra fruit; 22-26% (w/w), preferably about 24% (w/w) or about 95 mg Rosa canina fruit extract, which is equivalent to about 380 mg dry unmodified Rosa canina fruit; 8-12% (w/w), preferably about 10% (w/w) or about 40 mg Tumeric rhizome root extract, which is equivalent to about 1200 mg dry unmodified Tumeric rhizome root; 7-11% (w/w), preferably about 9% (w/w) or about 35 mg Vaccinium myrtillus extract, which is equivalent to about 675 mg dry unmodified Vaccinium myrtillus; and 6-10% (w/w), preferably about 8% (w/w) or about 33 mg Zingiber officinale root extract, which is equivalent to about 488 mg dry unmodified zingiber root officinale.

15. A method of treating or alleviating dysbiosis and/or loss of microbial diversity in the gut microbiota of a mammal, the method comprising the steps of:
administering a composition as defined in claim 1 to said mammal which composition comprises an extract comprising at least two plant secondary metabolites, which are encapsulated in an acid resistant coating; wherein said extract is a mixture of several extracts.

16. A process for the manufacture of a prebiotic composition for oral administration to a vertebrate animal comprising incorporating a composition as defined in claim 1, into said prebiotic composition.

17. A method of increasing or decreasing the relative population of one or more bacteria genus relative population of bacteria genus in the intestines of a vertebrate animal, the method comprising the steps of:
selecting a genus or a species of intestinal bacteria genus to be increased or decreased;
selecting at least two plant secondary metabolites able to increase or decrease said genus or species of intestinal bacteria,
providing a composition comprising an extract containing said at least two plant secondary metabolites, wherein said at least two plant secondary metabolites constitute at least 10% (w/w) by dry weight of the extract, wherein said at least two plant secondary metabolites are encapsulated in an acid resistant coating;
orally administering the composition to the vertebrate animal.

18. The method according to claim 17, wherein the genus of bacteria, for which and increase or decrease of abundancy is desired is selected from the group of bacteria genus consisting of: bifidobacterium, faecalibacterium, sutterella, bacteroides, rosburia, eubacterium, blautia, prevotella, dorea, ruminococcus, escherichia, enterobacter, coprococcus, veillonella, lactobacillus, streptococcus, bacillus, clostridium, salmonella, corynebacterium, holdemania, akkermansia, slackia, collinsella, methanobacterium, parabacteroides and christensenella.
